SEQUENCE LISTING (301 sequences)

SEQ ID NO: 1 - protein, 15 aa, Artificial Sequence
  SFTSV consensus glycoprotein Gn sequence region
MMKVIWFSSLICLVI

SEQ ID NO: 2 - protein, 15 aa, Artificial Sequence
  SFTSV consensus glycoprotein Gn sequence region
SSLICLVIQCSGDTG

SEQ ID NO: 3 - protein, 15 aa, Artificial Sequence
  SFTSV consensus glycoprotein Gn sequence region
IQCSGDTGPIICAGP

SEQ ID NO: 4 - protein, 15 aa, Artificial Sequence
  SFTSV consensus glycoprotein Gn sequence region
GPIICAGPIHSNKSA

SEQ ID NO: 5 - protein, 15 aa, Artificial Sequence
  SFTSV consensus glycoprotein Gn sequence region
PIHSNKSANIPHLLG

SEQ ID NO: 6 - protein, 15 aa, Artificial Sequence
  SFTSV consensus glycoprotein Gn sequence region
ANIPHLLGYSEKICQ

SEQ ID NO: 7 - protein, 15 aa, Artificial Sequence
  SFTSV consensus glycoprotein Gn sequence region
GYSEKICQIDRLIHV

SEQ ID NO: 8 - protein, 15 aa, Artificial Sequence
  SFTSV consensus glycoprotein Gn sequence region
QIDRLIHVSSWLRNH

SEQ ID NO: 9 - protein, 15 aa, Artificial Sequence
  SFTSV consensus glycoprotein Gn sequence region
VSSWLRNHSQFQGYV

SEQ ID NO: 10 - protein, 15 aa, Artificial Sequence
  SFTSV consensus glycoprotein Gn sequence region
HSQFQGYVGQRGGRS

SEQ ID NO: 11 - protein, 15 aa, Artificial Sequence
  SFTSV consensus glycoprotein Gn sequence region
VGQRGGRSQVSYYPA

SEQ ID NO: 12 - protein, 15 aa, Artificial Sequence
  SFTSV consensus glycoprotein Gn sequence region
SQVSYYPAENSYSRW

SEQ ID NO: 13 - protein, 15 aa, Artificial Sequence
  SFTSV consensus glycoprotein Gn sequence region
AENSYSRWSGLLSPC

SEQ ID NO: 14 - protein, 15 aa, Artificial Sequence
  SFTSV consensus glycoprotein Gn sequence region
WSGLLSPCDADWLGM

SEQ ID NO: 15 - protein, 15 aa, Artificial Sequence
  SFTSV consensus glycoprotein Gn sequence region
CDADWLGMLVVKKAK

SEQ ID NO: 16 - protein, 15 aa, Artificial Sequence
  SFTSV consensus glycoprotein Gn sequence region
MLVVKKAKGSDMIVP

SEQ ID NO: 17 - protein, 15 aa, Artificial Sequence
  SFTSV consensus glycoprotein Gn sequence region
KGSDMIVPGPSYKGK

SEQ ID NO: 18 - protein, 15 aa, Artificial Sequence
  SFTSV consensus glycoprotein Gn sequence region
PGPSYKGKVFFERPT

SEQ ID NO: 19 - protein, 15 aa, Artificial Sequence
  SFTSV consensus glycoprotein Gn sequence region
KVFFERPTFDGYVGW

SEQ ID NO: 20 - protein, 15 aa, Artificial Sequence
  SFTSV consensus glycoprotein Gn sequence region
TFDGYVGWGCGSGKS

SEQ ID NO: 21 - protein, 15 aa, Artificial Sequence
  SFTSV consensus glycoprotein Gn sequence region
WGCGSGKSRTESGEL

SEQ ID NO: 22 - protein, 15 aa, Artificial Sequence
  SFTSV consensus glycoprotein Gn sequence region
SRTESGELCSSDSGT

SEQ ID NO: 23 - protein, 15 aa, Artificial Sequence
  SFTSV consensus glycoprotein Gn sequence region
LCSSDSGTSSGLLPS

SEQ ID NO: 24 - protein, 15 aa, Artificial Sequence
  SFTSV consensus glycoprotein Gn sequence region
TSSGLLPSDRVLWIG

SEQ ID NO: 25 - protein, 15 aa, Artificial Sequence
  SFTSV consensus glycoprotein Gn sequence region
SDRVLWIGDVACQPM

SEQ ID NO: 26 - protein, 15 aa, Artificial Sequence
  SFTSV consensus glycoprotein Gn sequence region
GDVACQPMTPIPEET

SEQ ID NO: 27 - protein, 15 aa, Artificial Sequence
  SFTSV consensus glycoprotein Gn sequence region
MTPIPEETFLELKSF

SEQ ID NO: 28 - protein, 15 aa, Artificial Sequence
  SFTSV consensus glycoprotein Gn sequence region
TFLELKSFSQSEFPD

SEQ ID NO: 29 - protein, 15 aa, Artificial Sequence
  SFTSV consensus glycoprotein Gn sequence region
FSQSEFPDICKIDGI

SEQ ID NO: 30 - protein, 15 aa, Artificial Sequence
  SFTSV consensus glycoprotein Gn sequence region
DICKIDGIVFNQCEG

SEQ ID NO: 31 - protein, 15 aa, Artificial Sequence
  SFTSV consensus glycoprotein Gn sequence region
IVFNQCEGESLPQPF

SEQ ID NO: 32 - protein, 15 aa, Artificial Sequence
  SFTSV consensus glycoprotein Gn sequence region
GESLPQPFDVAWMDV

SEQ ID NO: 33 - protein, 15 aa, Artificial Sequence
  SFTSV consensus glycoprotein Gn sequence region
FDVAWMDVGHSHKII

SEQ ID NO: 34 - protein, 15 aa, Artificial Sequence
  SFTSV consensus glycoprotein Gn sequence region
VGHSHKIIMREHKTK

SEQ ID NO: 35 - protein, 15 aa, Artificial Sequence
  SFTSV consensus glycoprotein Gn sequence region
IMREHKTKWVQESSS

SEQ ID NO: 36 - protein, 15 aa, Artificial Sequence
  SFTSV consensus glycoprotein Gn sequence region
KWVQESSSKDFVCYK

SEQ ID NO: 37 - protein, 15 aa, Artificial Sequence
  SFTSV consensus glycoprotein Gn sequence region
SKDFVCYKEGTGPCS

SEQ ID NO: 38 - protein, 15 aa, Artificial Sequence
  SFTSV consensus glycoprotein Gn sequence region
KEGTGPCSESEEKTC

SEQ ID NO: 39 - protein, 15 aa, Artificial Sequence
  SFTSV consensus glycoprotein Gn sequence region
SESEEKTCKTSGSCR

SEQ ID NO: 40 - protein, 15 aa, Artificial Sequence
  SFTSV consensus glycoprotein Gn sequence region
CKTSGSCRGDMQFCK

SEQ ID NO: 41 - protein, 15 aa, Artificial Sequence
  SFTSV consensus glycoprotein Gn sequence region
RGDMQFCKVAGCEHG

SEQ ID NO: 42 - protein, 15 aa, Artificial Sequence
  SFTSV consensus glycoprotein Gn sequence region
KVAGCEHGEEASEAK

SEQ ID NO: 43 - protein, 15 aa, Artificial Sequence
  SFTSV consensus glycoprotein Gn sequence region
GEEASEAKCRCSLVH

SEQ ID NO: 44 - protein, 15 aa, Artificial Sequence
  SFTSV consensus glycoprotein Gn sequence region
KCRCSLVHKPGEVVV

SEQ ID NO: 45 - protein, 15 aa, Artificial Sequence
  SFTSV consensus glycoprotein Gn sequence region
HKPGEVVVSYGGMRV

SEQ ID NO: 46 - protein, 15 aa, Artificial Sequence
  SFTSV consensus glycoprotein Gn sequence region
VSYGGMRVRPKCYGF

SEQ ID NO: 47 - protein, 15 aa, Artificial Sequence
  SFTSV consensus glycoprotein Gn sequence region
VRPKCYGFSRMMATL

SEQ ID NO: 48 - protein, 15 aa, Artificial Sequence
  SFTSV consensus glycoprotein Gn sequence region
FSRMMATLEVNPPEQ

SEQ ID NO: 49 - protein, 15 aa, Artificial Sequence
  SFTSV consensus glycoprotein Gn sequence region
LEVNPPEQRIGQCTG

SEQ ID NO: 50 - protein, 15 aa, Artificial Sequence
  SFTSV consensus glycoprotein Gn sequence region
QRIGQCTGCHLECIN

SEQ ID NO: 51 - protein, 15 aa, Artificial Sequence
  SFTSV consensus glycoprotein Gn sequence region
GCHLECINGGVRLIT

SEQ ID NO: 52 - protein, 15 aa, Artificial Sequence
  SFTSV consensus glycoprotein Gn sequence region
NGGVRLITLTSELKS

SEQ ID NO: 53 - protein, 15 aa, Artificial Sequence
  SFTSV consensus glycoprotein Gn sequence region
TLTSELKSATVCASH

SEQ ID NO: 54 - protein, 15 aa, Artificial Sequence
  SFTSV consensus glycoprotein Gn sequence region
SATVCASHFCSSATS

SEQ ID NO: 55 - protein, 15 aa, Artificial Sequence
  SFTSV consensus glycoprotein Gn sequence region
HFCSSATSGKKSTEI

SEQ ID NO: 56 - protein, 15 aa, Artificial Sequence
  SFTSV consensus glycoprotein Gn sequence region
SGKKSTEIQFHSGSL

SEQ ID NO: 57 - protein, 15 aa, Artificial Sequence
  SFTSV consensus glycoprotein Gn sequence region
IQFHSGSLVGKTAIH

SEQ ID NO: 58 - protein, 15 aa, Artificial Sequence
  SFTSV consensus glycoprotein Gn sequence region
LVGKTAIHVKGALVD

SEQ ID NO: 59 - protein, 15 aa, Artificial Sequence
  SFTSV consensus glycoprotein Gn sequence region
HVKGALVDGTEFTFE

SEQ ID NO: 60 - protein, 15 aa, Artificial Sequence
  SFTSV consensus glycoprotein Gn sequence region
DGTEFTFEGSCMFPD

SEQ ID NO: 61 - protein, 15 aa, Artificial Sequence
  SFTSV consensus glycoprotein Gn sequence region
EGSCMFPDGCDAVDC

SEQ ID NO: 62 - protein, 15 aa, Artificial Sequence
  SFTSV consensus glycoprotein Gn sequence region
DGCDAVDCTFCREFL

SEQ ID NO: 63 - protein, 15 aa, Artificial Sequence
  SFTSV consensus glycoprotein Gn sequence region
CTFCREFLKNPQCYP

SEQ ID NO: 64 - protein, 15 aa, Artificial Sequence
  SFTSV consensus glycoprotein Gn sequence region
LKNPQCYPAKKWLFI

SEQ ID NO: 65 - protein, 15 aa, Artificial Sequence
  SFTSV consensus glycoprotein Gn sequence region
PAKKWLFIIIVILLG

SEQ ID NO: 66 - protein, 15 aa, Artificial Sequence
  SFTSV consensus glycoprotein Gn sequence region
IIIVILLGYAGLMLL

SEQ ID NO: 67 - protein, 15 aa, Artificial Sequence
  SFTSV consensus glycoprotein Gn sequence region
GYAGLMLLTNVLKAI

SEQ ID NO: 68 - protein, 15 aa, Artificial Sequence
  SFTSV consensus glycoprotein Gn sequence region
LTNVLKAIGVWGSWV

SEQ ID NO: 69 - protein, 15 aa, Artificial Sequence
  SFTSV consensus glycoprotein Gn sequence region
IGVWGSWVIAPVKLM

SEQ ID NO: 70 - protein, 15 aa, Artificial Sequence
  SFTSV consensus glycoprotein Gn sequence region
VIAPVKLMFAIIKKL

SEQ ID NO: 71 - protein, 15 aa, Artificial Sequence
  SFTSV consensus glycoprotein Gn sequence region
MFAIIKKLMRSVSCL

SEQ ID NO: 72 - protein, 15 aa, Artificial Sequence
  SFTSV consensus glycoprotein Gn sequence region
LMRSVSCLMGKLMDR

SEQ ID NO: 73 - protein, 15 aa, Artificial Sequence
  SFTSV consensus glycoprotein Gn sequence region
LMGKLMDRGRQVIHE

SEQ ID NO: 74 - protein, 15 aa, Artificial Sequence
  SFTSV consensus glycoprotein Gn sequence region
RGRQVIHEEIGENRE

SEQ ID NO: 75 - protein, 15 aa, Artificial Sequence
  SFTSV consensus glycoprotein Gn sequence region
EEIGENREGNQDDVR

SEQ ID NO: 76 - protein, 10 aa, Artificial Sequence
  SFTSV consensus glycoprotein Gn sequence region
EGNQDDVRIE

SEQ ID NO: 77 - protein, 15 aa, Artificial Sequence
  SFTSV consensus glycoprotein Gc sequence region
MARPRRVRHWMYSPV

SEQ ID NO: 78 - protein, 15 aa, Artificial Sequence
  SFTSV consensus glycoprotein Gc sequence region
RHWMYSPVILTILAI

SEQ ID NO: 79 - protein, 15 aa, Artificial Sequence
  SFTSV consensus glycoprotein Gc sequence region
VILTILAIGLAEGCD

SEQ ID NO: 80 - protein, 15 aa, Artificial Sequence
  SFTSV consensus glycoprotein Gc sequence region
IGLAEGCDEMVHADS

SEQ ID NO: 81 - protein, 15 aa, Artificial Sequence
  SFTSV consensus glycoprotein Gc sequence region
DEMVHADSKLVSCRQ

SEQ ID NO: 82 - protein, 15 aa, Artificial Sequence
  SFTSV consensus glycoprotein Gc sequence region
SKLVSCRQGSGNMKE

SEQ ID NO: 83 - protein, 15 aa, Artificial Sequence
  SFTSV consensus glycoprotein Gc sequence region
QGSGNMKECVTTGRA

SEQ ID NO: 84 - protein, 15 aa, Artificial Sequence
  SFTSV consensus glycoprotein Gc sequence region
ECVTTGRALLPAVNP

SEQ ID NO: 85 - protein, 15 aa, Artificial Sequence
  SFTSV consensus glycoprotein Gc sequence region
ALLPAVNPGQEACLH

SEQ ID NO: 86 - protein, 15 aa, Artificial Sequence
  SFTSV consensus glycoprotein Gc sequence region
PGQEACLHFTAPGSP

SEQ ID NO: 87 - protein, 15 aa, Artificial Sequence
  SFTSV consensus glycoprotein Gc sequence region
HFTAPGSPDSKCLKI

SEQ ID NO: 88 - protein, 15 aa, Artificial Sequence
  SFTSV consensus glycoprotein Gc sequence region
PDSKCLKIKVKRINL

SEQ ID NO: 89 - protein, 15 aa, Artificial Sequence
  SFTSV consensus glycoprotein Gc sequence region
IKVKRINLKCKKSSS

SEQ ID NO: 90 - protein, 15 aa, Artificial Sequence
  SFTSV consensus glycoprotein Gc sequence region
LKCKKSSSYFVPDAR

SEQ ID NO: 91 - protein, 15 aa, Artificial Sequence
  SFTSV consensus glycoprotein Gc sequence region
SYFVPDARSRCTSVR

SEQ ID NO: 92 - protein, 15 aa, Artificial Sequence
  SFTSV consensus glycoprotein Gc sequence region
RSRCTSVRRCRWAGD

SEQ ID NO: 93 - protein, 15 aa, Artificial Sequence
  SFTSV consensus glycoprotein Gc sequence region
RRCRWAGDCQSGCPP

SEQ ID NO: 94 - protein, 15 aa, Artificial Sequence
  SFTSV consensus glycoprotein Gc sequence region
DCQSGCPPHFTSNSF

SEQ ID NO: 95 - protein, 15 aa, Artificial Sequence
  SFTSV consensus glycoprotein Gc sequence region
PHFTSNSFSDDWAGK

SEQ ID NO: 96 - protein, 15 aa, Artificial Sequence
  SFTSV consensus glycoprotein Gc sequence region
FSDDWAGKMDRAGLG

SEQ ID NO: 97 - protein, 15 aa, Artificial Sequence
  SFTSV consensus glycoprotein Gc sequence region
KMDRAGLGFSGCSDG

SEQ ID NO: 98 - protein, 15 aa, Artificial Sequence
  SFTSV consensus glycoprotein Gc sequence region
GFSGCSDGCGGAACG

SEQ ID NO: 99 - protein, 15 aa, Artificial Sequence
  SFTSV consensus glycoprotein Gc sequence region
GCGGAACGCFNAAPS

SEQ ID NO: 100 - protein, 15 aa, Artificial Sequence
  SFTSV consensus glycoprotein Gc sequence region
GCFNAAPSCIFWRKW

SEQ ID NO: 101 - protein, 15 aa, Artificial Sequence
  SFTSV consensus glycoprotein Gc sequence region
SCIFWRKWVENPHGI

SEQ ID NO: 102 - protein, 15 aa, Artificial Sequence
  SFTSV consensus glycoprotein Gc sequence region
WVENPHGIIWKVSPC

SEQ ID NO: 103 - protein, 15 aa, Artificial Sequence
  SFTSV consensus glycoprotein Gc sequence region
IIWKVSPCAAWVPSA

SEQ ID NO: 104 - protein, 15 aa, Artificial Sequence
  SFTSV consensus glycoprotein Gc sequence region
CAAWVPSAVIELTMP

SEQ ID NO: 105 - protein, 15 aa, Artificial Sequence
  SFTSV consensus glycoprotein Gc sequence region
AVIELTMPSGEVRTF

SEQ ID NO: 106 - protein, 15 aa, Artificial Sequence
  SFTSV consensus glycoprotein Gc sequence region
PSGEVRTFHPMSGIP

SEQ ID NO: 107 - protein, 15 aa, Artificial Sequence
  SFTSV consensus glycoprotein Gc sequence region
FHPMSGIPTQVFKGV

SEQ ID NO: 108 - protein, 15 aa, Artificial Sequence
  SFTSV consensus glycoprotein Gc sequence region
PTQVFKGVSVTYLGS

SEQ ID NO: 109 - protein, 15 aa, Artificial Sequence
  SFTSV consensus glycoprotein Gc sequence region
VSVTYLGSDMEVSGL

SEQ ID NO: 110 - protein, 15 aa, Artificial Sequence
  SFTSV consensus glycoprotein Gc sequence region
SDMEVSGLTDLCEIE

SEQ ID NO: 111 - protein, 15 aa, Artificial Sequence
  SFTSV consensus glycoprotein Gc sequence region
LTDLCEIEELKSKKL

SEQ ID NO: 112 - protein, 15 aa, Artificial Sequence
  SFTSV consensus glycoprotein Gc sequence region
EELKSKKLALAPCNQ

SEQ ID NO: 113 - protein, 15 aa, Artificial Sequence
  SFTSV consensus glycoprotein Gc sequence region
LALAPCNQAGMGVVG

SEQ ID NO: 114 - protein, 15 aa, Artificial Sequence
  SFTSV consensus glycoprotein Gc sequence region
QAGMGVVGKVGEIQC

SEQ ID NO: 115 - protein, 15 aa, Artificial Sequence
  SFTSV consensus glycoprotein Gc sequence region
GKVGEIQCSSEESAR

SEQ ID NO: 116 - protein, 15 aa, Artificial Sequence
  SFTSV consensus glycoprotein Gc sequence region
CSSEESARTIKKDGC

SEQ ID NO: 117 - protein, 15 aa, Artificial Sequence
  SFTSV consensus glycoprotein Gc sequence region
RTIKKDGCIWNADLV

SEQ ID NO: 118 - protein, 15 aa, Artificial Sequence
  SFTSV consensus glycoprotein Gc sequence region
CIWNADLVGIELRVD

SEQ ID NO: 119 - protein, 15 aa, Artificial Sequence
  SFTSV consensus glycoprotein Gc sequence region
VGIELRVDDAVCYSK

SEQ ID NO: 120 - protein, 15 aa, Artificial Sequence
  SFTSV consensus glycoprotein Gc sequence region
DDAVCYSKITSVEAV

SEQ ID NO: 121 - protein, 15 aa, Artificial Sequence
  SFTSV consensus glycoprotein Gc sequence region
KITSVEAVANYSAIP

SEQ ID NO: 122 - protein, 15 aa, Artificial Sequence
  SFTSV consensus glycoprotein Gc sequence region
VANYSAIPTTIGGLR

SEQ ID NO: 123 - protein, 15 aa, Artificial Sequence
  SFTSV consensus glycoprotein Gc sequence region
PTTIGGLRFERSHDS

SEQ ID NO: 124 - protein, 15 aa, Artificial Sequence
  SFTSV consensus glycoprotein Gc sequence region
RFERSHDSQGKISGS

SEQ ID NO: 125 - protein, 15 aa, Artificial Sequence
  SFTSV consensus glycoprotein Gc sequence region
SQGKISGSPLDITAI

SEQ ID NO: 126 - protein, 15 aa, Artificial Sequence
  SFTSV consensus glycoprotein Gc sequence region
SPLDITAIRGSFSVN

SEQ ID NO: 127 - protein, 15 aa, Artificial Sequence
  SFTSV consensus glycoprotein Gc sequence region
IRGSFSVNYRGLRLS

SEQ ID NO: 128 - protein, 15 aa, Artificial Sequence
  SFTSV consensus glycoprotein Gc sequence region
NYRGLRLSLSEITAT

SEQ ID NO: 129 - protein, 15 aa, Artificial Sequence
  SFTSV consensus glycoprotein Gc sequence region
SLSEITATCTGEVTN

SEQ ID NO: 130 - protein, 15 aa, Artificial Sequence
  SFTSV consensus glycoprotein Gc sequence region
TCTGEVTNVSGCYSC

SEQ ID NO: 131 - protein, 15 aa, Artificial Sequence
  SFTSV consensus glycoprotein Gc sequence region
NVSGCYSCMTGAKVS

SEQ ID NO: 132 - protein, 15 aa, Artificial Sequence
  SFTSV consensus glycoprotein Gc sequence region
CMTGAKVSIKLHSSK

SEQ ID NO: 133 - protein, 15 aa, Artificial Sequence
  SFTSV consensus glycoprotein Gc sequence region
SIKLHSSKNSTAHVR

SEQ ID NO: 134 - protein, 15 aa, Artificial Sequence
  SFTSV consensus glycoprotein Gc sequence region
KNSTAHVRCKGDETA

SEQ ID NO: 135 - protein, 15 aa, Artificial Sequence
  SFTSV consensus glycoprotein Gc sequence region
RCKGDETAFSVLEGV

SEQ ID NO: 136 - protein, 15 aa, Artificial Sequence
  SFTSV consensus glycoprotein Gc sequence region
AFSVLEGVHSYTVSL

SEQ ID NO: 137 - protein, 15 aa, Artificial Sequence
  SFTSV consensus glycoprotein Gc sequence region
VHSYTVSLSFDHAVV

SEQ ID NO: 138 - protein, 15 aa, Artificial Sequence
  SFTSV consensus glycoprotein Gc sequence region
LSFDHAVVDEQCQLN

SEQ ID NO: 139 - protein, 15 aa, Artificial Sequence
  SFTSV consensus glycoprotein Gc sequence region
VDEQCQLNCGGHESQ

SEQ ID NO: 140 - protein, 15 aa, Artificial Sequence
  SFTSV consensus glycoprotein Gc sequence region
NCGGHESQVTLKGNL

SEQ ID NO: 141 - protein, 15 aa, Artificial Sequence
  SFTSV consensus glycoprotein Gc sequence region
QVTLKGNLIFLDVPK

SEQ ID NO: 142 - protein, 15 aa, Artificial Sequence
  SFTSV consensus glycoprotein Gc sequence region
LIFLDVPKFVDGSYM

SEQ ID NO: 143 - protein, 15 aa, Artificial Sequence
  SFTSV consensus glycoprotein Gc sequence region
KFVDGSYMQTYHSTV

SEQ ID NO: 144 - protein, 15 aa, Artificial Sequence
  SFTSV consensus glycoprotein Gc sequence region
MQTYHSTVPTGANIP

SEQ ID NO: 145 - protein, 15 aa, Artificial Sequence
  SFTSV consensus glycoprotein Gc sequence region
VPTGANIPSPTDWLN

SEQ ID NO: 146 - protein, 15 aa, Artificial Sequence
  SFTSV consensus glycoprotein Gc sequence region
PSPTDWLNALFGNGL

SEQ ID NO: 147 - protein, 15 aa, Artificial Sequence
  SFTSV consensus glycoprotein Gc sequence region
NALFGNGLSRWILGV

SEQ ID NO: 148 - protein, 15 aa, Artificial Sequence
  SFTSV consensus glycoprotein Gc sequence region
LSRWILGVIGVLLGG

SEQ ID NO: 149 - protein, 15 aa, Artificial Sequence
  SFTSV consensus glycoprotein Gc sequence region
VIGVLLGGLALFFLI

SEQ ID NO: 150 - protein, 15 aa, Artificial Sequence
  SFTSV consensus glycoprotein Gc sequence region
GLALFFLIMSLFKLG

SEQ ID NO: 151 - protein, 15 aa, Artificial Sequence
  SFTSV consensus glycoprotein Gc sequence region
IMSLFKLGTKQVFRS

SEQ ID NO: 152 - protein, 13 aa, Artificial Sequence
  SFTSV consensus glycoprotein Gc sequence region
GTKQVFRSRTKLA

SEQ ID NO: 153 - protein, 15 aa, Artificial Sequence
  SFTSV consensus glycoprotein NP sequence region
MSEWSRIAVEFGEQQ

SEQ ID NO: 154 - protein, 15 aa, Artificial Sequence
  SFTSV consensus glycoprotein NP sequence region
AVEFGEQQLNLTELE

SEQ ID NO: 155 - protein, 15 aa, Artificial Sequence
  SFTSV consensus glycoprotein NP sequence region
QLNLTELEDFARELA

SEQ ID NO: 156 - protein, 15 aa, Artificial Sequence
  SFTSV consensus glycoprotein NP sequence region
EDFARELAYEGLDPA

SEQ ID NO: 157 - protein, 15 aa, Artificial Sequence
  SFTSV consensus glycoprotein NP sequence region
AYEGLDPALIIKKLK

SEQ ID NO: 158 - protein, 15 aa, Artificial Sequence
  SFTSV consensus glycoprotein NP sequence region
ALIIKKLKETGGDDW

SEQ ID NO: 159 - protein, 15 aa, Artificial Sequence
  SFTSV consensus glycoprotein NP sequence region
KETGGDDWVKDTKFI

SEQ ID NO: 160 - protein, 15 aa, Artificial Sequence
  SFTSV consensus glycoprotein NP sequence region
WVKDTKFIIVFALTR

SEQ ID NO: 161 - protein, 15 aa, Artificial Sequence
  SFTSV consensus glycoprotein NP sequence region
IIVFALTRGNKIVKA

SEQ ID NO: 162 - protein, 15 aa, Artificial Sequence
  SFTSV consensus glycoprotein NP sequence region
RGNKIVKASGKMSNS

SEQ ID NO: 163 - protein, 15 aa, Artificial Sequence
  SFTSV consensus glycoprotein NP sequence region
ASGKMSNSGSKRLMA

SEQ ID NO: 164 - protein, 15 aa, Artificial Sequence
  SFTSV consensus glycoprotein NP sequence region
SGSKRLMALQEKYGL

SEQ ID NO: 165 - protein, 15 aa, Artificial Sequence
  SFTSV consensus glycoprotein NP sequence region
ALQEKYGLVERAETR

SEQ ID NO: 166 - protein, 15 aa, Artificial Sequence
  SFTSV consensus glycoprotein NP sequence region
LVERAETRLSITPVR

SEQ ID NO: 167 - protein, 15 aa, Artificial Sequence
  SFTSV consensus glycoprotein NP sequence region
RLSITPVRVAQSLPT

SEQ ID NO: 168 - protein, 15 aa, Artificial Sequence
  SFTSV consensus glycoprotein NP sequence region
RVAQSLPTWTCAAAA

SEQ ID NO: 169 - protein, 15 aa, Artificial Sequence
  SFTSV consensus glycoprotein NP sequence region
TWTCAAAAALKEYLP

SEQ ID NO: 170 - protein, 15 aa, Artificial Sequence
  SFTSV consensus glycoprotein NP sequence region
AALKEYLPVGPAVMN

SEQ ID NO: 171 - protein, 15 aa, Artificial Sequence
  SFTSV consensus glycoprotein NP sequence region
PVGPAVMNLKVENYP

SEQ ID NO: 172 - protein, 15 aa, Artificial Sequence
  SFTSV consensus glycoprotein NP sequence region
NLKVENYPPEMMCMA

SEQ ID NO: 173 - protein, 15 aa, Artificial Sequence
  SFTSV consensus glycoprotein NP sequence region
PPEMMCMAFGSLIPT

SEQ ID NO: 174 - protein, 15 aa, Artificial Sequence
  SFTSV consensus glycoprotein NP sequence region
AFGSLIPTAGVSEAT

SEQ ID NO: 175 - protein, 15 aa, Artificial Sequence
  SFTSV consensus glycoprotein NP sequence region
TAGVSEATTKTLMEA

SEQ ID NO: 176 - protein, 15 aa, Artificial Sequence
  SFTSV consensus glycoprotein NP sequence region
TTKTLMEAYSLWQDA

SEQ ID NO: 177 - protein, 15 aa, Artificial Sequence
  SFTSV consensus glycoprotein NP sequence region
AYSLWQDAFTKTINV

SEQ ID NO: 178 - protein, 15 aa, Artificial Sequence
  SFTSV consensus glycoprotein NP sequence region
AFTKTINVKMRGASK

SEQ ID NO: 179 - protein, 15 aa, Artificial Sequence
  SFTSV consensus glycoprotein NP sequence region
VKMRGASKTEVYNSF

SEQ ID NO: 180 - protein, 15 aa, Artificial Sequence
  SFTSV consensus glycoprotein NP sequence region
KTEVYNSFRDPLHAA

SEQ ID NO: 181 - protein, 15 aa, Artificial Sequence
  SFTSV consensus glycoprotein NP sequence region
FRDPLHAAVNSVFFP

SEQ ID NO: 182 - protein, 15 aa, Artificial Sequence
  SFTSV consensus glycoprotein NP sequence region
AVNSVFFPNDVRVKW

SEQ ID NO: 183 - protein, 15 aa, Artificial Sequence
  SFTSV consensus glycoprotein NP sequence region
PNDVRVKWLKAKGIL

SEQ ID NO: 184 - protein, 15 aa, Artificial Sequence
  SFTSV consensus glycoprotein NP sequence region
WLKAKGILGPDGVPS

SEQ ID NO: 185 - protein, 15 aa, Artificial Sequence
  SFTSV consensus glycoprotein NP sequence region
LGPDGVPSRAAEVAA

SEQ ID NO: 186 - protein, 14 aa, Artificial Sequence
  SFTSV consensus glycoprotein NP sequence region
SRAAEVAAAAYRNL

SEQ ID NO: 187 - protein, 15 aa, Artificial Sequence
  SFTSV consensus glycoprotein NS sequence region
MSLSKCSNVDLKSVA

SEQ ID NO: 188 - protein, 15 aa, Artificial Sequence
  SFTSV consensus glycoprotein NS sequence region
NVDLKSVAMNANTVR

SEQ ID NO: 189 - protein, 15 aa, Artificial Sequence
  SFTSV consensus glycoprotein NS sequence region
AMNANTVRLEPSLGE

SEQ ID NO: 190 - protein, 15 aa, Artificial Sequence
  SFTSV consensus glycoprotein NS sequence region
RLEPSLGEYPTLRRD

SEQ ID NO: 191 - protein, 15 aa, Artificial Sequence
  SFTSV consensus glycoprotein NS sequence region
EYPTLRRDLVECSCS

SEQ ID NO: 192 - protein, 15 aa, Artificial Sequence
  SFTSV consensus glycoprotein NS sequence region
DLVECSCSVLTLSMV

SEQ ID NO: 193 - protein, 15 aa, Artificial Sequence
  SFTSV consensus glycoprotein NS sequence region
SVLTLSMVKRMGKMT

SEQ ID NO: 194 - protein, 15 aa, Artificial Sequence
  SFTSV consensus glycoprotein NS sequence region
VKRMGKMTNTVWLFG

SEQ ID NO: 195 - protein, 15 aa, Artificial Sequence
  SFTSV consensus glycoprotein NS sequence region
TNTVWLFGNPKNPLH

SEQ ID NO: 196 - protein, 15 aa, Artificial Sequence
  SFTSV consensus glycoprotein NS sequence region
GNPKNPLHQLEPGLE

SEQ ID NO: 197 - protein, 15 aa, Artificial Sequence
  SFTSV consensus glycoprotein NS sequence region
HQLEPGLEQLLDMYY

SEQ ID NO: 198 - protein, 15 aa, Artificial Sequence
  SFTSV consensus glycoprotein NS sequence region
EQLLDMYYKDMRCYS

SEQ ID NO: 199 - protein, 15 aa, Artificial Sequence
  SFTSV consensus glycoprotein NS sequence region
YKDMRCYSQRELSAL

SEQ ID NO: 200 - protein, 15 aa, Artificial Sequence
  SFTSV consensus glycoprotein NS sequence region
SQRELSALRWPSGKP

SEQ ID NO: 201 - protein, 15 aa, Artificial Sequence
  SFTSV consensus glycoprotein NS sequence region
LRWPSGKPSVWFLQA

SEQ ID NO: 202 - protein, 15 aa, Artificial Sequence
  SFTSV consensus glycoprotein NS sequence region
PSVWFLQAAHMFFSI

SEQ ID NO: 203 - protein, 15 aa, Artificial Sequence
  SFTSV consensus glycoprotein NS sequence region
AAHMFFSIKNSWAME

SEQ ID NO: 204 - protein, 15 aa, Artificial Sequence
  SFTSV consensus glycoprotein NS sequence region
IKNSWAMETGRENWR

SEQ ID NO: 205 - protein, 15 aa, Artificial Sequence
  SFTSV consensus glycoprotein NS sequence region
ETGRENWRGLFHRIT

SEQ ID NO: 206 - protein, 15 aa, Artificial Sequence
  SFTSV consensus glycoprotein NS sequence region
RGLFHRITKGQKYLF

SEQ ID NO: 207 - protein, 15 aa, Artificial Sequence
  SFTSV consensus glycoprotein NS sequence region
TKGQKYLFEGDMILD

SEQ ID NO: 208 - protein, 15 aa, Artificial Sequence
  SFTSV consensus glycoprotein NS sequence region
FEGDMILDSLEAIEK

SEQ ID NO: 209 - protein, 15 aa, Artificial Sequence
  SFTSV consensus glycoprotein NS sequence region
DSLEAIEKRRLRLGL

SEQ ID NO: 210 - protein, 15 aa, Artificial Sequence
  SFTSV consensus glycoprotein NS sequence region
KRRLRLGLPEILITG

SEQ ID NO: 211 - protein, 15 aa, Artificial Sequence
  SFTSV consensus glycoprotein NS sequence region
LPEILITGLSPILDV

SEQ ID NO: 212 - protein, 15 aa, Artificial Sequence
  SFTSV consensus glycoprotein NS sequence region
GLSPILDVALLQIES

SEQ ID NO: 213 - protein, 15 aa, Artificial Sequence
  SFTSV consensus glycoprotein NS sequence region
VALLQIESLARLRGM

SEQ ID NO: 214 - protein, 15 aa, Artificial Sequence
  SFTSV consensus glycoprotein NS sequence region
SLARLRGMSLNHHLF

SEQ ID NO: 215 - protein, 15 aa, Artificial Sequence
  SFTSV consensus glycoprotein NS sequence region
MSLNHHLFTSSSLRK

SEQ ID NO: 216 - protein, 15 aa, Artificial Sequence
  SFTSV consensus glycoprotein NS sequence region
FTSSSLRKPLLDCWD

SEQ ID NO: 217 - protein, 15 aa, Artificial Sequence
  SFTSV consensus glycoprotein NS sequence region
KPLLDCWDFFIPIRK

SEQ ID NO: 218 - protein, 15 aa, Artificial Sequence
  SFTSV consensus glycoprotein NS sequence region
DFFIPIRKKKTDGSY

SEQ ID NO: 219 - protein, 15 aa, Artificial Sequence
  SFTSV consensus glycoprotein NS sequence region
KKKTDGSYSVLDEDD

SEQ ID NO: 220 - protein, 15 aa, Artificial Sequence
  SFTSV consensus glycoprotein NS sequence region
YSVLDEDDEPGVLQG

SEQ ID NO: 221 - protein, 15 aa, Artificial Sequence
  SFTSV consensus glycoprotein NS sequence region
DEPGVLQGYPYLMAH

SEQ ID NO: 222 - protein, 15 aa, Artificial Sequence
  SFTSV consensus glycoprotein NS sequence region
GYPYLMAHYLNRCPF

SEQ ID NO: 223 - protein, 15 aa, Artificial Sequence
  SFTSV consensus glycoprotein NS sequence region
HYLNRCPFHNLIRFD

SEQ ID NO: 224 - protein, 15 aa, Artificial Sequence
  SFTSV consensus glycoprotein NS sequence region
FHNLIRFDEELRTAA

SEQ ID NO: 225 - protein, 15 aa, Artificial Sequence
  SFTSV consensus glycoprotein NS sequence region
DEELRTAALNTIWGR

SEQ ID NO: 226 - protein, 15 aa, Artificial Sequence
  SFTSV consensus glycoprotein NS sequence region
ALNTIWGRDWPAIGD

SEQ ID NO: 227 - protein, 13 aa, Artificial Sequence
  SFTSV consensus glycoprotein NS sequence region
RDWPAIGDLPKEV

SEQ ID NO: 228 - protein, 15 aa, Artificial Sequence
  SFTSV consensus RNA dependent RNA polymerase RdRp sequence region
MNLEVLCGRINVENG

SEQ ID NO: 229 - protein, 15 aa, Artificial Sequence
  SFTSV consensus RNA dependent RNA polymerase RdRp sequence region
GRINVENGLSLGEPG

SEQ ID NO: 230 - protein, 15 aa, Artificial Sequence
  SFTSV consensus RNA dependent RNA polymerase RdRp sequence region
GLSLGEPGLYDQIYD

SEQ ID NO: 231 - protein, 15 aa, Artificial Sequence
  SFTSV consensus RNA dependent RNA polymerase RdRp sequence region
GLYDQIYDRPGLPDL

SEQ ID NO: 232 - protein, 15 aa, Artificial Sequence
  SFTSV consensus RNA dependent RNA polymerase RdRp sequence region
DRPGLPDLDVTVDAT

SEQ ID NO: 233 - protein, 15 aa, Artificial Sequence
  SFTSV consensus RNA dependent RNA polymerase RdRp sequence region
LDVTVDATGVTVDIG

SEQ ID NO: 234 - protein, 15 aa, Artificial Sequence
  SFTSV consensus RNA dependent RNA polymerase RdRp sequence region
TGVTVDIGAVPDSAS

SEQ ID NO: 235 - protein, 15 aa, Artificial Sequence
  SFTSV consensus RNA dependent RNA polymerase RdRp sequence region
GAVPDSASQLGSSIN

SEQ ID NO: 236 - protein, 15 aa, Artificial Sequence
  SFTSV consensus RNA dependent RNA polymerase RdRp sequence region
SQLGSSINAGLITIQ

SEQ ID NO: 237 - protein, 15 aa, Artificial Sequence
  SFTSV consensus RNA dependent RNA polymerase RdRp sequence region
NAGLITIQLSEAYKI

SEQ ID NO: 238 - protein, 15 aa, Artificial Sequence
  SFTSV consensus RNA dependent RNA polymerase RdRp sequence region
QLSEAYKINHDFTFS

SEQ ID NO: 239 - protein, 15 aa, Artificial Sequence
  SFTSV consensus RNA dependent RNA polymerase RdRp sequence region
INHDFTFSGLSKTTD

SEQ ID NO: 240 - protein, 15 aa, Artificial Sequence
  SFTSV consensus RNA dependent RNA polymerase RdRp sequence region
SGLSKTTDRRLSEVF

SEQ ID NO: 241 - protein, 15 aa, Artificial Sequence
  SFTSV consensus RNA dependent RNA polymerase RdRp sequence region
DRRLSEVFPITHDGS

SEQ ID NO: 242 - protein, 15 aa, Artificial Sequence
  SFTSV consensus RNA dependent RNA polymerase RdRp sequence region
FPITHDGSDGMTPDV

SEQ ID NO: 243 - protein, 15 aa, Artificial Sequence
  SFTSV consensus RNA dependent RNA polymerase RdRp sequence region
SDGMTPDVIHTRLDG

SEQ ID NO: 244 - protein, 15 aa, Artificial Sequence
  SFTSV consensus RNA dependent RNA polymerase RdRp sequence region
VIHTRLDGTIVVVEF

SEQ ID NO: 245 - protein, 15 aa, Artificial Sequence
  SFTSV consensus RNA dependent RNA polymerase RdRp sequence region
GTIVVVEFSTTRSHN

SEQ ID NO: 246 - protein, 15 aa, Artificial Sequence
  SFTSV consensus RNA dependent RNA polymerase RdRp sequence region
FSTTRSHNIGGLEAA

SEQ ID NO: 247 - protein, 15 aa, Artificial Sequence
  SFTSV consensus RNA dependent RNA polymerase RdRp sequence region
NIGGLEAAYRTKIEK

SEQ ID NO: 248 - protein, 15 aa, Artificial Sequence
  SFTSV consensus RNA dependent RNA polymerase RdRp sequence region
AYRTKIEKYRDPISR

SEQ ID NO: 249 - protein, 15 aa, Artificial Sequence
  SFTSV consensus RNA dependent RNA polymerase RdRp sequence region
KYRDPISRRVDIMEN

SEQ ID NO: 250 - protein, 15 aa, Artificial Sequence
  SFTSV consensus RNA dependent RNA polymerase RdRp sequence region
RRVDIMENPRVFFGV

SEQ ID NO: 251 - protein, 15 aa, Artificial Sequence
  SFTSV consensus RNA dependent RNA polymerase RdRp sequence region
NPRVFFGVIVVSSGG

SEQ ID NO: 252 - protein, 15 aa, Artificial Sequence
  SFTSV consensus RNA dependent RNA polymerase RdRp sequence region
VIVVSSGGVLSNMPL

SEQ ID NO: 253 - protein, 15 aa, Artificial Sequence
  SFTSV consensus RNA dependent RNA polymerase RdRp sequence region
GVLSNMPLTQDEAEE

SEQ ID NO: 254 - protein, 15 aa, Artificial Sequence
  SFTSV consensus RNA dependent RNA polymerase RdRp sequence region
LTQDEAEELMYRFCI

SEQ ID NO: 255 - protein, 15 aa, Artificial Sequence
  SFTSV consensus RNA dependent RNA polymerase RdRp sequence region
ELMYRFCIANEIYTK

SEQ ID NO: 256 - protein, 15 aa, Artificial Sequence
  SFTSV consensus RNA dependent RNA polymerase RdRp sequence region
IANEIYTKARSMDAD

SEQ ID NO: 257 - protein, 15 aa, Artificial Sequence
  SFTSV consensus RNA dependent RNA polymerase RdRp sequence region
KARSMDADIELQKSE

SEQ ID NO: 258 - protein, 15 aa, Artificial Sequence
  SFTSV consensus RNA dependent RNA polymerase RdRp sequence region
DIELQKSEEELEAIS

SEQ ID NO: 259 - protein, 15 aa, Artificial Sequence
  SFTSV consensus RNA dependent RNA polymerase RdRp sequence region
EEELEAISRALSFFS

SEQ ID NO: 260 - protein, 15 aa, Artificial Sequence
  SFTSV consensus RNA dependent RNA polymerase RdRp sequence region
SRALSFFSLFEPNIE

SEQ ID NO: 261 - protein, 15 aa, Artificial Sequence
  SFTSV consensus RNA dependent RNA polymerase RdRp sequence region
SLFEPNIERVEGTFP

SEQ ID NO: 262 - protein, 15 aa, Artificial Sequence
  SFTSV consensus RNA dependent RNA polymerase RdRp sequence region
ERVEGTFPNSEIEML

SEQ ID NO: 263 - protein, 15 aa, Artificial Sequence
  SFTSV consensus RNA dependent RNA polymerase RdRp sequence region
PNSEIEMLEQFLSTP

SEQ ID NO: 264 - protein, 15 aa, Artificial Sequence
  SFTSV consensus RNA dependent RNA polymerase RdRp sequence region
LEQFLSTPADVDFIT

SEQ ID NO: 265 - protein, 15 aa, Artificial Sequence
  SFTSV consensus RNA dependent RNA polymerase RdRp sequence region
PADVDFITKTLKAKE

SEQ ID NO: 266 - protein, 15 aa, Artificial Sequence
  SFTSV consensus RNA dependent RNA polymerase RdRp sequence region
TKTLKAKEVEAYADL

SEQ ID NO: 267 - protein, 15 aa, Artificial Sequence
  SFTSV consensus RNA dependent RNA polymerase RdRp sequence region
EVEAYADLCDSHYLK

SEQ ID NO: 268 - protein, 15 aa, Artificial Sequence
  SFTSV consensus RNA dependent RNA polymerase RdRp sequence region
LCDSHYLKPEKTIQE

SEQ ID NO: 269 - protein, 15 aa, Artificial Sequence
  SFTSV consensus RNA dependent RNA polymerase RdRp sequence region
KPEKTIQERLEINRC

SEQ ID NO: 270 - protein, 15 aa, Artificial Sequence
  SFTSV consensus RNA dependent RNA polymerase RdRp sequence region
ERLEINRCEAIDKTQ

SEQ ID NO: 271 - protein, 15 aa, Artificial Sequence
  SFTSV consensus RNA dependent RNA polymerase RdRp sequence region
CEAIDKTQDLLAGLH

SEQ ID NO: 272 - protein, 15 aa, Artificial Sequence
  SFTSV consensus RNA dependent RNA polymerase RdRp sequence region
QDLLAGLHARSNKQT

SEQ ID NO: 273 - protein, 15 aa, Artificial Sequence
  SFTSV consensus RNA dependent RNA polymerase RdRp sequence region
HARSNKQTSLNRGTV

SEQ ID NO: 274 - protein, 15 aa, Artificial Sequence
  SFTSV consensus RNA dependent RNA polymerase RdRp sequence region
TSLNRGTVKLPPWLP

SEQ ID NO: 275 - protein, 15 aa, Artificial Sequence
  SFTSV consensus RNA dependent RNA polymerase RdRp sequence region
VKLPPWLPKPSSESI

SEQ ID NO: 276 - protein, 15 aa, Artificial Sequence
  SFTSV consensus RNA dependent RNA polymerase RdRp sequence region
PKPSSESIDIKTDSG

SEQ ID NO: 277 - protein, 15 aa, Artificial Sequence
  SFTSV consensus RNA dependent RNA polymerase RdRp sequence region
IDIKTDSGFGSLMDH

SEQ ID NO: 278 - protein, 15 aa, Artificial Sequence
  SFTSV consensus RNA dependent RNA polymerase RdRp sequence region
GFGSLMDHGAYGELW

SEQ ID NO: 279 - protein, 15 aa, Artificial Sequence
  SFTSV consensus RNA dependent RNA polymerase RdRp sequence region
HGAYGELWAKCLLDV

SEQ ID NO: 280 - protein, 15 aa, Artificial Sequence
  SFTSV consensus RNA dependent RNA polymerase RdRp sequence region
WAKCLLDVSLGNVEG

SEQ ID NO: 281 - protein, 15 aa, Artificial Sequence
  SFTSV consensus RNA dependent RNA polymerase RdRp sequence region
VSLGNVEGVVSDPAK

SEQ ID NO: 282 - protein, 15 aa, Artificial Sequence
  SFTSV consensus RNA dependent RNA polymerase RdRp sequence region
GVVSDPAKELDIAIS

SEQ ID NO: 283 - protein, 15 aa, Artificial Sequence
  SFTSV consensus RNA dependent RNA polymerase RdRp sequence region
KELDIAISDDPEKDT

SEQ ID NO: 284 - protein, 15 aa, Artificial Sequence
  SFTSV consensus RNA dependent RNA polymerase RdRp sequence region
SDDPEKDTPKEAKIT

SEQ ID NO: 285 - protein, 15 aa, Artificial Sequence
  SFTSV consensus RNA dependent RNA polymerase RdRp sequence region
TPKEAKITYRRFKPA

SEQ ID NO: 286 - DNA, 1611 bp, Artificial Sequence
  SFTSV Gn
atgatgaaggtcatctggttcagcagcctgatctgcctggtcatccagtgcagcggcgacaccggccccatcatctgtgccggccctatccacagcaacaagagcgccaacatcccccatctgctgggctacagcgagaagatctgccagatcgaccggctgatccacgtgtccagctggctgcggaaccacagccagttccagggctacgtgggccagagaggcggcagatcccaggtgtcctactaccccgccgagaacagctacagccggtggtccggcctgctgagcccttgcgacgccgattggctgggcatgctggtggtcaagaaggccaagggcagcgacatgatcgtgcctggccccagctacaagggcaaggtgttcttcgagcggcccaccttcgacggctatgtgggctggggctgtggcagcggcaagagcagaaccgagagcggcgagctgtgcagctccgacagcggcacaagcagcggcctgctgcccagcgacagagtgctgtggatcggcgacgtggcctgccagcccatgacccctatccccgaggaaaccttcctggaactgaagtccttcagccagagcgagttccccgacatctgcaagatcgatggcatcgtgttcaaccagtgcgagggcgagagcctgccccagcccttcgatgtggcctggatggacgtgggccacagccacaagatcatcatgcgcgagcacaagaccaaatgggtgcaggaaagcagcagcaaggacttcgtgtgctacaaagagggcaccggcccctgcagcgagagcgaggaaaagacctgcaagaccagcggcagctgcagaggcgacatgcagttctgcaaggtggccggctgcgagcacggcgaagaggccagcgaggccaagtgcagatgcagcctggtgcacaagcccggcgaggtggtggtgtcctacggcggcatgagagtgcggcccaagtgctacggcttcagccggatgatggccaccctggaagtgaacccccccgagcagagaatcggccagtgcaccggctgccacctggaatgcatcaacggcggcgtgcggctgatcaccctgaccagcgagctgaagtccgccaccgtgtgcgccagccacttctgcagcagcgccacctccggcaagaagtccaccgagatccagttccacagcggcagcctcgtgggcaagaccgccatccacgtgaagggcgccctggtggacggcaccgagttcaccttcgagggcagctgcatgttccccgacggctgcgacgccgtggactgtaccttctgcagagagttcctgaagaacccccagtgctaccctgccaagaagtggctgttcatcatcatcgtgatcctgctgggatacgccggcctgatgctgctgaccaacgtgctgaaggccatcggcgtgtggggcagctgggtgatcgcccccgtgaagctgatgttcgccatcatcaagaaactgatgcggagcgtgtcctgcctgatgggcaagctgatggaccggggcagacaggtcatccacgaggaaatcggcgagaacagagagggcaaccaggacgacgtgcggatcgagtgatga

SEQ ID NO: 287 - protein, 535 aa, Artificial Sequence
  SFTSV Gn
MMKVIWFSSLICLVIQCSGDTGPIICAGPIHSNKSANIPHLLGYSEKICQIDRLIHVSSWLRNHSQFQGYVGQRGGRSQVSYYPAENSYSRWSGLLSPCDADWLGMLVVKKAKGSDMIVPGPSYKGKVFFERPTFDGYVGWGCGSGKSRTESGELCSSDSGTSSGLLPSDRVLWIGDVACQPMTPIPEETFLELKSFSQSEFPDICKIDGIVFNQCEGESLPQPFDVAWMDVGHSHKIIMREHKTKWVQESSSKDFVCYKEGTGPCSESEEKTCKTSGSCRGDMQFCKVAGCEHGEEASEAKCRCSLVHKPGEVVVSYGGMRVRPKCYGFSRMMATLEVNPPEQRIGQCTGCHLECINGGVRLITLTSELKSATVCASHFCSSATSGKKSTEIQFHSGSLVGKTAIHVKGALVDGTEFTFEGSCMFPDGCDAVDCTFCREFLKNPQCYPAKKWLFIIIVILLGYAGLMLLTNVLKAIGVWGSWVIAPVKLMFAIIKKLMRSVSCLMGKLMDRGRQVIHEEIGENREGNQDDVRIE

SEQ ID NO: 288 - DNA, 1620 bp, Artificial Sequence
  SFTSV Gc
atggccagacctagaagagtgcggcactggatgtacagccccgtgatcctgaccatcctggccatcggcctggccgagggctgcgacgaaatggtgcacgccgacagcaagctggtgtcctgcagacagggcagcggcaacatgaaggaatgcgtgaccaccggcagagccctgctgcccgccgtgaatccaggccaggaagcctgcctgcacttcaccgcccctggcagccccgactccaagtgcctgaagatcaaagtgaagcggatcaacctgaagtgcaagaagtccagcagctacttcgtgcccgacgcccggtccagatgcaccagcgtgcggagatgcagatgggctggcgactgccagagcggctgcccccctcacttcaccagcaacagcttcagcgacgactgggccggcaagatggacagagccggcctgggcttcagcggctgttccgatggatgtggcggagccgcctgcggctgcttcaatgccgcccctagctgcatcttctggcggaaatgggtggaaaacccccacggcatcatctggaaggtgtccccctgtgccgcctgggtgccaagcgccgtgatcgagctgaccatgcccagcggcgaagtgcggaccttccaccccatgagcggcatccccacccaggtgttcaagggcgtgtccgtgacctacctgggcagcgacatggaagtgtccggcctgaccgacctgtgcgagatcgaggaactgaagtctaagaagctggccctggccccctgcaaccaggccggaatgggcgtcgtgggcaaagtgggcgagatccagtgcagcagcgaggaaagcgcccggaccatcaagaaggacggctgcatctggaacgccgacctcgtgggcatcgagctgagagtggacgacgccgtgtgctacagcaagatcacctccgtggaagccgtggccaactactccgccatccccaccaccatcggcggcctgagattcgagcggagccacgacagccagggcaagatcagcggcagccccctggacatcaccgccatcagaggcagcttctccgtgaactaccggggcctgagactgagcctgagcgagatcaccgccacctgtaccggcgaagtgaccaacgtgtccggctgctacagctgcatgaccggcgccaaggtgtccatcaagctgcacagcagcaagaacagcaccgcccacgtgcggtgcaagggcgacgagacagccttcagcgtgctggaaggcgtgcacagctacaccgtgtccctgagcttcgaccacgccgtggtggacgagcagtgccagctgaactgtggcggccacgagtcccaggtcacactgaagggcaacctgatcttcctggacgtgcccaagttcgtggacggcagctacatgcagacctaccacagcaccgtgcccacaggcgccaacatccccagccccaccgactggctgaacgccctgttcggcaacggcctgagccggtggattctgggcgtgatcggcgtgctgctgggcggcctggccctgttcttcctgatcatgagcctgttcaagctgggcaccaaacaggtgttcagaagccggaccaagctggcctgatga

SEQ ID NO: 289 - protein, 538 aa, Artificial Sequence
  SFTSV Gc
MARPRRVRHWMYSPVILTILAIGLAEGCDEMVHADSKLVSCRQGSGNMKECVTTGRALLPAVNPGQEACLHFTAPGSPDSKCLKIKVKRINLKCKKSSSYFVPDARSRCTSVRRCRWAGDCQSGCPPHFTSNSFSDDWAGKMDRAGLGFSGCSDGCGGAACGCFNAAPSCIFWRKWVENPHGIIWKVSPCAAWVPSAVIELTMPSGEVRTFHPMSGIPTQVFKGVSVTYLGSDMEVSGLTDLCEIEELKSKKLALAPCNQAGMGVVGKVGEIQCSSEESARTIKKDGCIWNADLVGIELRVDDAVCYSKITSVEAVANYSAIPTTIGGLRFERSHDSQGKISGSPLDITAIRGSFSVNYRGLRLSLSEITATCTGEVTNVSGCYSCMTGAKVSIKLHSSKNSTAHVRCKGDETAFSVLEGVHSYTVSLSFDHAVVDEQCQLNCGGHESQVTLKGNLIFLDVPKFVDGSYMQTYHSTVPTGANIPSPTDWLNALFGNGLSRWILGVIGVLLGGLALFFLIMSLFKLGTKQVFRSRTKLA

SEQ ID NO: 290 - DNA, 741 bp, Artificial Sequence
  SFTSV NP
atgagcgagtggtcccggatcgccgtggaattcggcgagcagcagctgaacctgaccgagctggaagatttcgccagagagctggcctacgagggcctggaccccgccctgatcatcaagaagctgaaagagacaggcggcgacgactgggtcaaggacaccaagttcatcatcgtgttcgccctgacccggggcaacaagatcgtgaaggccagcggcaagatgagcaacagcggcagcaagcggctgatggccctgcaggagaagtacggcctggtcgagcgggccgagacacggctgtctatcacacctgtgcgggtggcccagagcctgcctacctggacatgtgccgccgctgccgccctgaaagaatacctgcctgtgggccctgccgtgatgaacctgaaggtggaaaactacccccccgagatgatgtgcatggccttcggcagcctgatccccacagccggcgtgtccgaggccaccaccaagaccctgatggaagcctacagcctgtggcaggacgccttcaccaagaccatcaacgtgaagatgagaggcgccagcaagaccgaggtgtacaacagcttccgggaccccctgcacgccgccgtgaacagcgtgttcttccccaacgacgtgcgcgtgaagtggctgaaggccaagggcatcctgggccccgatggcgtgccatctagagccgccgaagtggccgcagccgcctacagaaacctgtgatga

SEQ ID NO: 291 - protein, 245 aa, Artificial Sequence
  SFTSV NP
MSEWSRIAVEFGEQQLNLTELEDFARELAYEGLDPALIIKKLKETGGDDWVKDTKFIIVFALTRGNKIVKASGKMSNSGSKRLMALQEKYGLVERAETRLSITPVRVAQSLPTWTCAAAAALKEYLPVGPAVMNLKVENYPPEMMCMAFGSLIPTAGVSEATTKTLMEAYSLWQDAFTKTINVKMRGASKTEVYNSFRDPLHAAVNSVFFPNDVRVKWLKAKGILGPDGVPSRAAEVAAAAYRNL

SEQ ID NO: 292 - DNA, 885 bp, Artificial Sequence
  SFTSV NS
atgagcctgagcaagtgcagcaacgtggacctgaagtccgtggccatgaacgccaacaccgtgcggctggaacccagcctgggcgagtaccccaccctgcggagagatctggtggaatgcagctgcagcgtgctgaccctgagcatggtcaagcggatgggcaagatgaccaataccgtgtggctgttcggcaaccccaagaaccccctgcaccagctggaacctggcctggaacagctgctggacatgtactacaaggacatgcggtgctacagccagagagagctgagcgccctgcggtggcctagcggcaagcccagcgtgtggtttctgcaagccgcccacatgttcttcagcatcaagaacagctgggccatggaaaccggcagagagaattggagaggcctgttccaccggatcaccaagggccagaagtacctgttcgagggcgacatgatcctggacagcctggaagccatcgagaagcggcggctgagactgggcctgcccgagatcctgatcaccggcctgagccccatcctggacgtggccctgctgcagatcgagagcctggcccggctgcggggcatgtccctgaaccaccacctgttcaccagcagcagcctgcggaagcccctgctggactgctgggacttcttcatccccatccggaagaagaaaaccgacggcagctactccgtgctggacgaggacgacgagcctggcgtgctgcagggctacccctacctgatggcccactacctgaaccggtgccccttccacaacctgatcagattcgacgaggaactgcggacagccgccctgaacaccatctggggcagagactggcccgccatcggcgacctgcccaaagaagtgtgatga

SEQ ID NO: 293 - protein, 293 aa, Artificial Sequence
  SFTSV NS
MSLSKCSNVDLKSVAMNANTVRLEPSLGEYPTLRRDLVECSCSVLTLSMVKRMGKMTNTVWLFGNPKNPLHQLEPGLEQLLDMYYKDMRCYSQRELSALRWPSGKPSVWFLQAAHMFFSIKNSWAMETGRENWRGLFHRITKGQKYLFEGDMILDSLEAIEKRRLRLGLPEILITGLSPILDVALLQIESLARLRGMSLNHHLFTSSSLRKPLLDCWDFFIPIRKKKTDGSYSVLDEDDEPGVLQGYPYLMAHYLNRCPFHNLIRFDEELRTAALNTIWGRDWPAIGDLPKEV

SEQ ID NO: 294 - DNA, 6258 bp, Artificial Sequence
  SFTSV RdRp
atgaacctggaagtgctgtgcggccggatcaacgtggaaaacggcctgagcctgggcgagcccggcctgtacgaccagatctacgacagacccggcctgcccgacctggacgtgacagtggatgccaccggcgtgaccgtggacatcggcgccgtgcctgatagcgccagccagctgggcagcagcatcaacgccggcctgatcaccatccagctgagcgaggcctacaagatcaaccacgacttcaccttcagcggcctgagcaagaccaccgaccggcggctgtccgaggtgttccccatcacccacgacggcagcgacggcatgacccccgacgtgatccacacccggctggacggcaccatcgtggtggtggaattcagcaccaccagaagccacaacatcggcggcctggaagccgcctaccggaccaagatcgagaagtaccgggaccccatcagcagacgggtggacatcatggaaaacccccgggtgttcttcggcgtgatcgtggtgtcctctggcggcgtgctgagcaacatgcccctgacccaggacgaggccgaggaactgatgtaccggttctgtatcgccaacgagatctacaccaaggcccggtccatggacgccgacatcgagctgcagaagtccgaggaagaactggaagccatcagcagggccctgagcttcttcagcctgttcgagcccaacatcgagcgggtggaaggcaccttccccaacagcgagatcgagatgctggaacagttcctgagcacccctgccgacgtggacttcatcaccaagaccctgaaggccaaagaggtggaagcctacgccgacctgtgcgacagccactacctgaagcccgagaaaaccatccaagagcggctggaaatcaacagatgcgaggccatcgacaagacccaggacctgctggccggcctgcacgccagaagcaacaagcagaccagcctgaaccggggcaccgtgaagctgcccccctggctgcctaagcccagcagcgagagcatcgacatcaagaccgacagcggcttcggcagcctgatggaccacggcgcctatggcgagctgtgggccaagtgcctgctggacgtgtccctgggcaacgtggaaggcgtggtgtccgaccccgccaaagagctggatatcgccatcagcgacgaccccgagaaggacacccccaaagaggccaagatcacctacagacggttcaagcccgccctgagcagcagcgccagacaagagttcagcctgcaaggcgtggaaggcaagaaatggaagcggatggccgccaaccagaagaaagagaaagagtcccacgaaaccctgagcccattcctggacgtggaagatatcggcgacttcctgaccttcaacaatctgctggccgacagcagatacggcgacgagagcgtgcagcgggccgtgtccatcctgctggaaaaggccagcgccatgcaggacaccgagctgacccacgccctgaacgacagcttcaagcggaacctgtccagcaacgtggtgcagtggtccctgtgggtgtcctgcctggcccaagagctggcctctgccctgaagcagcactgcagagccggcgagttcatcatcaagaagctgaagttctggcccatctacgtgatcatcaagcccaccaagagcagctcccacatcttctacagcctgggcatccggaaggccgacgtgaccagacggctgaccggcagagtgttcagcgacaccatcgacgccggcgagtgggagctgaccgagttcaagagcctgaaaacctgcaagctgaccaacctggtcaacctgccctgcaccatgctgaactctatcgccttctggcgcgagaagctgggcgtggccccttggctcgtgcggaagccttgcagcgagctgcgcgagcaagtgggcctgaccttcctgatcagcctggaagataagagcaagaccgaggaaatcatcaccctgacccggtacacccagatggaaggcttcgtgtcccccccaatgctgcccaagccccagaagatgctgggcaagctggacggccccctgcggaccaagctgcaggtctacctgctgcggaagcacctggactgcatggtccgaatcgcctcccagcccttcagcctgatcccccgcgagggcagagtggaatggggcggcacctttcacgccatctccggcagaagcaccaacctggaaaacatggtcaacagctggtacatcggctactacaagaacaaagaggaaagcacagagctgaacgccctgggcgagatgtacaagaaaatcgtggaaatggaagaggacaagcccagctcccccgagttcctgggctggggcgacaccgacagccccaagaagcacgagttcagcagaagcttcctgagagccgcctgcagcagcctggaaagagagatcgcccagcggcacggccggcagtggaagcagaatctggaagagagagtgctgagagagatcggcaccaagaacatcctggacctggccagcatgaaggccaccagcaacttcagcaaggactgggagctgtacagcgaggtgcagaccaaagagtaccacagaagcaagctgctcgagaagatggccaccctgatcgagaagggcgtgatgtggtacatcgatgccgtgggccaggcctggaaggccgtgctggacgacggctgcatgcggatctgcctgttcaagaagaaccagcacggcggcctgcgcgagatctatgtgatggacgccaacgcccggctggtgcagttcggcgtggaaaccatggccagatgcgtgtgcgagctgagcccccacgagacagtggccaaccccagactgaagaacagcatcatcgagaaccacggcctgaagtccgccagaagcctgggccctggctccatcaacatcaacagcagcaacgacgccaagaagtggaaccagggccactacaccaccaagctggccctggtgctgtgctggttcatgcccgccaagttccaccggttcatctgggccgccatcagcatgttccggcggaagaaaatgatggtggacctgcggttcctggcccacctgtcctccaagagcgagagcagaagcagcgaccccttccgcgaggccatgaccgatgccttccacggcaaccgcgaggtgtcctggatggacaagggccggacctacatcaagacagagacaggcatgatgcagggcatcctgcacttcaccagcagcctgctgcacagctgcgtgcagagcttctacaagagctacttcgtgtccaagctgaaagagggctacatgggcgagtccatcagcggcgtggtggacgtgatcgagggcagcgacgacagcgccatcatgatcagcatcagacccaagagcgacatggacgaagtgcggagccggttcttcgtggccaacctgctgcactccgtgaagtttctgaaccccctgttcggcatctacagctccgagaagtccaccgtgaacaccgtgtactgcgtcgagtacaacagcgagttccacttccaccggcacctcgtgcggcccaccctgagatggattgccgccagccaccagatcagcgagacagaggccctggccagccggcaagaggactacagcaacctgctgacccagtgtctggaaggcggagccagcttctccctgacatacctgatccagtgcgcccagctgctgcaccactacatgctgctgggcctgtgcctgcaccctctgtttggcaccttcatgggcatgctgatcagcgaccccgaccctgccctgggcttctttctgatggacaaccccgcctttgccggcggagccggctttcggtttaacctgtggcgggcctgtaaaaccaccgacctgggccggaagtacgcctactacttcaacgagatccagggcaagaccaagggcgacgaggactaccgggccctggatgccacaagcggcggcacactgagccacagcgtgatggtgtactggggcgaccggaagaagtaccaggccctgctgaaccggatgggcctgcctgaggactgggtggaacagatcgacgagaaccccggcgtgctgtacagaagggccgccaacaaaaaagagctgctgctgaagctggccgagaaggtgcacagccctggcgtgaccagctccctgagcaagggccacgtggtgcccagagtggtggctgccggcgtgtacctgctgagcagacactgcttccggttcagctccagcatccacggcagaggcagcgcccagaaggccagcctgatcaagctgctgatgatgagcagcattagcgccatgaagcacggcggaagcctgaaccccaatcaagagcggatgctgttcccccaagcccaagagtacgacagagtgtgcaccctgctggaagaggtcgagcacctgaccggcaagttcgtcgtgcgcgagcggaacatcgtgcggagcagaatcgacctgttccaagagcccgtggacctgagatgcaaggccgaggacctggtgtccgaagtttggttcggcctgaagcggacaaagctgggccccagactgctgaaagaagagtgggacaagctgcgggccagcttcgcctggctgagcaccgaccctagcgaaaccctgcgggacggccccttcctgtcccacgtgcagtttcggaactttatcgcccacgtggacgccaagagccgcagcgtcagactgctgggagcccccgtgaagaaaagcggcggagtgaccaccatcagccaggtcgtgcggatgaacttcttccccggattctctctggaagccgagaagtctctggacaatcaagagagactggaatccatctccatcctgaagcacgtgctgttcatggtgctgaacggcccctacaccgaagagtacaagctggaaatgatcatcgaggccttcagcaccctggtcatcccccagcccagcgaagtgatccggaagtcccggaccatgaccctgtgtctgctgtccaactacctgagcagccggggaggcagcatcctggatcagatcgagagagcccagagcggcaccctgggcggcttcagcaagcctcagaaaaccttcatcagacccggcggaggcatcggctacaagggcaagggcgtgtggaccggcgtgatggaagatacacacgtgcagatcctgatcgacggcgacggcacctccaactggctggaagagatccggctgagcagcgacgccaggctgtacgatgtgatcgagagcatcagacggctgtgcgacgacctgggcatcaacaaccgggtggccagcgcctaccggggccactgtatggtccgactgagcggcttcaagatcaagcctgccagccggaccgacggctgccccgtgcggatcatggaacggggcttcagaatccgggaactgcagaaccccgacgaagtgaagatgagagtgcggggcgacatcctgaacctgagcgtgacaatccaagagggccgcgtgatgaacatcctgagctaccggcccagagacaccgacatcagcgagagcgccgctgcctacctgtggtccaaccgggacctgttcagcttcggcaagaaagagccctcctgcagctggatctgtctgaaaaccctggacaactgggcctggtcccacgccagcgtgctgctggccaacgacagaaagacccagggcatcgacaacagagccatgggcaacatcttccgggactgcctcgagggctccctgagaaagcagggcctgatgcggagcaagctgacagagatggtggaaaagaacgtggtgcctctgaccacacaagagctggtggatatcctggaagaggatatcgatttcagcgacgtgatcgccgtggaactgagcgagggcagcctggacatcgagtccatcttcgatggcgcccctatcctttggagcgccgaggtggaagagttcggcgagggcgtggtggctgtgtcctacagcagcaagtactaccacctgaccctgatggatcaggccgccattaccatgtgtgccatcatgggcaaagagggctgcagaggcctgctgaccgagaagcggtgtatggccgccatccgggaacaagtgcggcctttcctgatcttcctgcagatccccgaggacagcatcagctgggtgtccgatcagttctgcgacagcaggggcctggacgaggaatccaccatcatgtggggctgatga

SEQ ID NO: 295 - protein, 2084 aa, Artificial Sequence
  SFTSV RdRp
MNLEVLCGRINVENGLSLGEPGLYDQIYDRPGLPDLDVTVDATGVTVDIGAVPDSASQLGSSINAGLITIQLSEAYKINHDFTFSGLSKTTDRRLSEVFPITHDGSDGMTPDVIHTRLDGTIVVVEFSTTRSHNIGGLEAAYRTKIEKYRDPISRRVDIMENPRVFFGVIVVSSGGVLSNMPLTQDEAEELMYRFCIANEIYTKARSMDADIELQKSEEELEAISRALSFFSLFEPNIERVEGTFPNSEIEMLEQFLSTPADVDFITKTLKAKEVEAYADLCDSHYLKPEKTIQERLEINRCEAIDKTQDLLAGLHARSNKQTSLNRGTVKLPPWLPKPSSESIDIKTDSGFGSLMDHGAYGELWAKCLLDVSLGNVEGVVSDPAKELDIAISDDPEKDTPKEAKITYRRFKPALSSSARQEFSLQGVEGKKWKRMAANQKKEKESHETLSPFLDVEDIGDFLTFNNLLADSRYGDESVQRAVSILLEKASAMQDTELTHALNDSFKRNLSSNVVQWSLWVSCLAQELASALKQHCRAGEFIIKKLKFWPIYVIIKPTKSSSHIFYSLGIRKADVTRRLTGRVFSDTIDAGEWELTEFKSLKTCKLTNLVNLPCTMLNSIAFWREKLGVAPWLVRKPCSELREQVGLTFLISLEDKSKTEEIITLTRYTQMEGFVSPPMLPKPQKMLGKLDGPLRTKLQVYLLRKHLDCMVRIASQPFSLIPREGRVEWGGTFHAISGRSTNLENMVNSWYIGYYKNKEESTELNALGEMYKKIVEMEEDKPSSPEFLGWGDTDSPKKHEFSRSFLRAACSSLEREIAQRHGRQWKQNLEERVLREIGTKNILDLASMKATSNFSKDWELYSEVQTKEYHRSKLLEKMATLIEKGVMWYIDAVGQAWKAVLDDGCMRICLFKKNQHGGLREIYVMDANARLVQFGVETMARCVCELSPHETVANPRLKNSIIENHGLKSARSLGPGSININSSNDAKKWNQGHYTTKLALVLCWFMPAKFHRFIWAAISMFRRKKMMVDLRFLAHLSSKSESRSSDPFREAMTDAFHGNREVSWMDKGRTYIKTETGMMQGILHFTSSLLHSCVQSFYKSYFVSKLKEGYMGESISGVVDVIEGSDDSAIMISIRPKSDMDEVRSRFFVANLLHSVKFLNPLFGIYSSEKSTVNTVYCVEYNSEFHFHRHLVRPTLRWIAASHQISETEALASRQEDYSNLLTQCLEGGASFSLTYLIQCAQLLHHYMLLGLCLHPLFGTFMGMLISDPDPALGFFLMDNPAFAGGAGFRFNLWRACKTTDLGRKYAYYFNEIQGKTKGDEDYRALDATSGGTLSHSVMVYWGDRKKYQALLNRMGLPEDWVEQIDENPGVLYRRAANKKELLLKLAEKVHSPGVTSSLSKGHVVPRVVAAGVYLLSRHCFRFSSSIHGRGSAQKASLIKLLMMSSISAMKHGGSLNPNQERMLFPQAQEYDRVCTLLEEVEHLTGKFVVRERNIVRSRIDLFQEPVDLRCKAEDLVSEVWFGLKRTKLGPRLLKEEWDKLRASFAWLSTDPSETLRDGPFLSHVQFRNFIAHVDAKSRSVRLLGAPVKKSGGVTTISQVVRMNFFPGFSLEAEKSLDNQERLESISILKHVLFMVLNGPYTEEYKLEMIIEAFSTLVIPQPSEVIRKSRTMTLCLLSNYLSSRGGSILDQIERAQSGTLGGFSKPQKTFIRPGGGIGYKGKGVWTGVMEDTHVQILIDGDGTSNWLEEIRLSSDARLYDVIESIRRLCDDLGINNRVASAYRGHCMVRLSGFKIKPASRTDGCPVRIMERGFRIRELQNPDEVKMRVRGDILNLSVTIQEGRVMNILSYRPRDTDISESAAAYLWSNRDLFSFGKKEPSCSWICLKTLDNWAWSHASVLLANDRKTQGIDNRAMGNIFRDCLEGSLRKQGLMRSKLTEMVEKNVVPLTTQELVDILEEDIDFSDVIAVELSEGSLDIESIFDGAPILWSAEVEEFGEGVVAVSYSSKYYHLTLMDQAAITMCAIMGKEGCRGLLTEKRCMAAIREQVRPFLIFLQIPEDSISWVSDQFCDSRGLDEESTIMWG

SEQ ID NO: 296 - protein, 177 aa, Artificial Sequence
  Synthetic peptide
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 297 - protein, 264 aa, Artificial Sequence
  Synthetic peptide
MRPRMKYSNSKISPAKCDSTSGRALVPPCKITRSQQKTKDICHVYCMRLRSGLTIRKETCYFGKEPAKRYSLKSGSKHEGRLSTCLPDSRKRSLLGSIQAFAASVDTLSIQGTSLLTESCALSTYNDQSVSFVLENGCYVINVEDCGKNQEKDKVLLRYYESSFPAQSGDGVDGKKLMVNMSPIKDTDIWLNANDKDYSVELQKGDVSPPDQAFFVLHKKSSDFVSFECKNLPGTYIGVKDNQLALVEENDESCNNIMFKLSKM

SEQ ID NO: 298 - DNA, 468 bp, Artificial Sequence
  Synthetic nucleic acid
atgttccacgtgtccttccggtacatcttcggcatcccccccctgatcctggtgctgctgcctgtgaccagcagcgagtgccacatcaaggacaaagagggcaaggcctacgagagcgtgctgatgatcagcatcgacgagctggacaagatgaccggcaccgacagcaactgccccaacaacgagcccaacttcttcagaaagcacgtgtgcgacgataccaaagaggccgccttcctgaacagagccgccagaaagctgaagcagttcctgaagatgaacatcagcgaggaattcaacgtgcacctcctgaccgtgtcccagggcacccagaccctggtcaactgcaccagcaaagaggaaaagaacgtcaaagagcagaagaagaacgacgcctgcttcctgaagagactgctgagagagatcaagacctgctggaacaagatcctgaagggcagcatctgatga

SEQ ID NO: 299 - DNA, 804 bp, Artificial Sequence
  Synthetic nucleic acid
atgaggcccagaatgaagtacagcaacagcaagatcagccccgccaagttcagcagcacagccggcgaggctctggtgcccccctgcaagatcagaagaagccagcagaaaacaaaagagttctgccacgtctactgcatgagactgagaagcggcctgaccatcagaaaagagacaagctacttccggaaagagcccaccaagagatacagcctgaagtccggcaccaagcacgaggaaaacttcagcgcctaccccagagacagcagaaagagaagcctgctgggcagcatccaggccttcgccgccagcgtggacaccctgagcatccagggcaccagcctgctgacccagagccctgccagcctgagcacctacaacgaccagagcgtgtccttcgtgctggaaaacggctgctacgtgatcaacgtggacgacagcggcaaggaccaggaacaggaccaagtcctgctgaggtactacgagagcccctgcccagccagccagtctggggatggcgtggacggcaagaaactgatggtcaacatgagccccatcaaggacaccgacatctggctgcacgccaacgacaaggactacagcgtggaactgcagaggggcgacgtgtcccccccagagcaggccttcttcgtgctgcacaagaagtccagcgacttcgtcagcttcgagtgcaagaacctgcccggcacctacatcggcgtgaaggacaaccagctggccctggtggaagagaaggacgagagctgcaacaacatcatgttcaagctgagcaagatctgatga

SEQ ID NO: 300 - DNA, 542 bp, Artificial Sequence
  Synthetic nucleic acid
ggatccgccaccatggactggacctggattctgttcctggtggccgctgccacccgggtgcacagcatgttccacgtgtccttccggtacatcttcggcatcccccccctgatcctggtgctgctgcctgtgaccagcagcgagtgccacatcaaggacaaagagggcaaggcctacgagagcgtgctgatgatcagcatcgacgagctggacaagatgaccggcaccgacagcaactgccccaacaacgagcccaacttcttcagaaagcacgtgtgcgacgataccaaagaggccgccttcctgaacagagccgccagaaagctgaagcagttcctgaagatgaacatcagcgaggaattcaacgtgcacctcctgaccgtgtcccagggcacccagaccctggtcaactgcaccagcaaagaggaaaagaacgtcaaagagcagaagaagaacgacgcctgcttcctgaagagactgctgagagagatcaagacctgctggaacaagatcctgaagggcagcatctgatgagcggccgc

SEQ ID NO: 301 - DNA, 878 bp, Artificial Sequence
  Synthetic nucleic acid
ggatccgccaccatggactggacctggattctgttcctggtggccgctgccacccgggtgcacagcatgaggcccagaatgaagtacagcaacagcaagatcagccccgccaagttcagcagcacagccggcgaggctctggtgcccccctgcaagatcagaagaagccagcagaaaacaaaagagttctgccacgtctactgcatgagactgagaagcggcctgaccatcagaaaagagacaagctacttccggaaagagcccaccaagagatacagcctgaagtccggcaccaagcacgaggaaaacttcagcgcctaccccagagacagcagaaagagaagcctgctgggcagcatccaggccttcgccgccagcgtggacaccctgagcatccagggcaccagcctgctgacccagagccctgccagcctgagcacctacaacgaccagagcgtgtccttcgtgctggaaaacggctgctacgtgatcaacgtggacgacagcggcaaggaccaggaacaggaccaagtcctgctgaggtactacgagagcccctgcccagccagccagtctggggatggcgtggacggcaagaaactgatggtcaacatgagccccatcaaggacaccgacatctggctgcacgccaacgacaaggactacagcgtggaactgcagaggggcgacgtgtcccccccagagcaggccttcttcgtgctgcacaagaagtccagcgacttcgtcagcttcgagtgcaagaacctgcccggcacctacatcggcgtgaaggacaaccagctggccctggtggaagagaaggacgagagctgcaacaacatcatgttcaagctgagcaagatctgatgagcggccgc

(19) United States
(12) Patent Application Publication
(10) Pub. No.: US 2021/0220464 A1
(43) Pub. Date: Jul. 22, 2021

(54) Vaccine Composition for Preventing or Treating Diseases Caused by Severe Fever with Thrombocytopenia Syndrome (SFTS) Viral Infection

(71) Applicants: Korea Advanced Institute of Science and Technology, Daejeon (KR); Geneone Life Science, Inc., Seoul (KR)

(72) Inventors: Su Hyung Park, Daejeon (KR); Jeong Eun Kwak, Incheon (KR); Moon Sup Jeong, Incheon (KR); Jin Ah Kwon, Seoul (KR); Hyo Jin Lee, Gyeonggi-do (KR); Young Ran Cho, Seoul (KR); Ji Hye Koo, Gyeonggi-do (KR); Joel Maslow, Blue Bell, PA (US); Byung Mun Cho, Incheon (KR); Young Kun Park, Blue Bell, PA (US)

(21) Appl. No.: 17/256,547

(22) PCT Filed: Jan. 2, 2020
(86) PCT No.: PCT/KR2019/007920

§ 371 Date: Dec. 28, 2020

(30) Foreign Application Priority Data
Jun. 28, 2018 (KR) 10-2018-0074418

(51) Int. Cl. A61K 39/12; A61K 9/00; A61K 41/00; A61K 39/39; A61P 31/14; C12N 15/85

(52) U.S. Cl. CPC A61K 39/12; A61K 9/0019; A61K 41/0047; A61K 2039/54; A61P 31/14; C12N 15/85; A61K 39/39

(57) ABSTRACT

The present disclosure relates to a vaccine composition for preventing or treating infectious diseases caused by severe fever with thrombocytopenia syndrome (SFTS) virus.

### TECHNICAL FIELD

The present disclosure relates to a vaccine composition for preventing or treating an infectious disease caused by severe fever with thrombocytopenia syndrome (SFTS) virus.

### BACKGROUND ART

Severe fever with thrombocytopenia syndrome (SFTS) is an emerging viral disease that is endemic in China, Korea and Japan. No effective vaccine or specific treatment for SFTS is currently available. SFTS is a severe disease that causes symptoms such as high fever, vomiting, diarrhea, thrombopenia, leukopenia, and multiple organ failure, and has a mortality rate of 6 to 30% (Yu X J et al., N. Engl. J. Med. 2011; 364:1523-32; Ding F et al., Clin Infect Dis 2013: 56:1682-3).

In addition, seroconversion and viremia of SFTS virus have been found in domesticated animals such as goats, sheep, cattle, pigs and dogs, and these animals have been implicated as intermediate hosts in SFTS virus-endemic areas (Zhao L et al., Emerg Infect Dis 2013; 18: 963-5: Niu G et al., Emerg Infect Dis 2013; 19: 756-63).

Meanwhile, Chinese Patent Application Publication No. 102070704 discloses a kit for amplifying and detecting SFTS virus.

Accordingly, the present inventors have developed an effective vaccine against SFTS.

### DISCLOSURE

### Technical Problem

An object of the present disclosure is to provide a recombinant DNA or peptide of SFTS virus antigen that effectively induces an immune response in a subject, and an SFTS virus vaccine comprising the same.

Specifically, the present disclosure is intended to provide a vaccine composition for preventing or treating an infectious disease caused by severe fever with thrombocytopenia syndrome (SFTS) virus.

However, technical objects to be achieved by the present disclosure are not limited to the above-mentioned object, and other technical objects which are not mentioned herein will be clearly understood by those skilled in the art from the following description.

### Technical Solution

Hereinafter, various embodiments described herein will be described with reference to the drawings. In the following description, numerous specific details are set forth, such as specific configurations, compositions, and processes, etc., in order to provide a thorough understanding of the present disclosure. However, certain embodiments may be practiced without one or more of these specific details, or in combination with other known methods and configurations. In other instances, known processes and preparation techniques have not been described in particular detail in order to not unnecessarily obscure the present disclosure. Reference throughout this specification to “one embodiment” or “an embodiment” means that a particular feature, configuration, composition, or characteristic described in connection with the embodiment is included in at least one embodiment of the present disclosure. Thus, the appearances of the phrase “in one embodiment” or “an embodiment” in various places throughout this specification do not necessarily refer to the same embodiment of the present disclosure. Additionally, the particular features, configurations, compositions, or characteristics may be combined in any suitable manner in one or more embodiments.

Unless otherwise stated in the specification, all the scientific and technical tenns used in the specification have the same meanings as commonly understood by those skilled in the technical field to which the present disclosure pertains.

As used herein, the term “preventing” refer to any action of delaying viral growth, proliferation, invasion or infection by administering the composition of the present disclosure.

As used herein, the terms “treating” and “alleviating” refer to any action of alleviating or beneficially changing SFTSV-related diseases by suppressing viral growth, proliferation or infection through administration of the composition of the present disclosure.

The present disclosure relates to a virus vaccine comprising a recombinant DNA or peptide of virus antigen and a vaccination method using the same, and also to improvement in a vaccination method comprising a step of introducing into a mammalian body a nucleotide sequence encoding an immunogen which is an antigenic protein or peptide (where the protein or peptide is expressed in the mammalian body, causing an immune response against the antigenic protein or peptide). This vaccination method is known.

According to one embodiment of the present disclosure, there is provided an antigenic composition comprising, as an active ingredient, any one or more recombinant peptides selected from the group consisting of:

a first recombinant peptide which comprises an amino acid sequence represented by SEQ ID NO: 287, or which is encoded by a first recombinant DNA comprising a nucleotide sequence represented by SEQ ID NO: 286:

a second recombinant peptide which comprises an amino acid sequence represented by SEQ ID NO: 289, or which is encoded by a second recombinant DNA comprising a nucleotide sequence represented by SEQ ID NO: 288;

a third recombinant peptide which comprises an amino acid sequence represented by SEQ ID NO: 291, or which is encoded by a third recombinant DNA comprising a nucleotide sequence represented by SEQ ID NO: 290:

a fourth recombinant peptide which comprises an amino acid sequence represented by SEQ ID NO: 293, or which is encoded by a fourth recombinant DNA comprising a nucleotide sequence represented by SEQ ID NO: 292; and

a fifth recombinant peptide which comprises an amino acid sequence represented by SEQ ID NO: 295, or which is encoded by a fifth recombinant DNA comprising a nucleotide sequence represented by SEQ ID NO: 294.

The antigenic composition of the present disclosure may comprise at least one of: the first recombinant peptide which comprises the amino acid sequence represented by SEQ ID NO: 287, or which is encoded by the first recombinant DNA comprising the nucleotide sequence represented by SEQ ID NO: 286; and the second recombinant peptide which comprises the amino acid sequence represented by SEQ ID NO: 289, or which is encoded by the second recombinant DNA comprising the nucleotide sequence represented by SEQ ID NO: 288.

The antigenic composition of the present disclosure may comprise: the first recombinant peptide which comprises the amino acid sequence represented by SEQ ID NO: 287, or which is encoded by the first recombinant DNA comprising the nucleotide sequence represented by SEQ ID NO: 286; and the second recombinant peptide which comprises the amino acid sequence represented by SEQ ID NO: 289, or which is encoded by the second recombinant DNA comprising the nucleotide sequence represented by SEQ ID NO: 288.

The antigenic composition of the present disclosure may comprise a peptide represented by the amino acid sequence of at least one of SEQ ID NOs: 1 to 76.

The antigenic composition of the present disclosure may comprise a peptide represented by the amino acid sequence of at least one of SEQ ID NOs: 1 to 38.

The antigenic composition of the present disclosure may comprise a peptide represented by the amino acid sequence of at least one of SEQ ID NOs: 39 to 76.

The antigenic composition of the present disclosure may comprise: a peptide represented by the amino acid sequence of at least one of SEQ ID NOs: 1 to 38; and a peptide represented by the amino acid sequence of at least one of SEQ ID NOs: 39 to 76.

The antigenic composition of the present disclosure may comprise a peptide represented by the amino acid sequence of at least one of SEQ ID NOs: 77 to 152.

The antigenic composition of the present disclosure may comprise a peptide represented by the amino acid sequence of at least one of SEQ ID NOs: 77 to 114.

The antigenic composition of the present disclosure may comprise a peptide represented by the amino acid sequence of at least one of SEQ ID NOs: 115 to 152.

The antigenic composition of the present disclosure may comprise: a peptide represented by the amino acid sequence of at least one of SEQ ID NOs: 77 to 114; and a peptide represented by the amino acid sequence of at least one of SEQ ID NOs: 115 to 152.

The antigenic composition of the present disclosure may comprise a peptide represented by the amino acid sequence of at least one of SEQ ID NOs: 153 to 186.

The antigenic composition of the present disclosure may comprise a peptide represented by the amino acid sequence of at least one of SEQ ID NOs: 187 to 227.

The antigenic composition of the present disclosure may comprise a peptide represented by the amino acid sequence of at least one of SEQ ID NOs: 187 to 207.

The antigenic composition of the present disclosure may comprise a peptide represented by the amino acid sequence of at least one of SEQ ID NOs: 208 to 227.

The antigenic composition of the present disclosure may comprise: a peptide represented by the amino acid sequence of at least one of SEQ ID NOs: 187 to 207; and a peptide represented by the amino acid sequence of at least one of SEQ ID NOs: 208 to 227.

The antigenic composition of the present disclosure may comprise a peptide represented by the amino acid sequence of at least one of SEQ ID NOs: 228 to 285.

The antigenic composition of the present disclosure may comprise a peptide represented by the amino acid sequence of at least one of SEQ ID NOs: 228 to 256.

The antigenic composition of the present disclosure may comprise a peptide represented by the amino acid sequence of at least one of SEQ ID NOs: 257 to 285.

The antigenic composition of the present disclosure may comprise: a peptide represented by the amino acid sequence of at least one of SEQ ID NOs: 228 to 256; and a peptide represented by the amino acid sequence of at least one of SEQ ID NOs: 257 to 285.

In the present disclosure, the antigenic composition is injected in vivo through a route selected from among intramuscular, intradermal, subcutaneous, subepidermal, transdermal and intravenous routes, but the route is not limited thereto. In the present disclosure, the antigenic composition is injected into a subject through intramuscular injection. In the present disclosure, the antigenic composition is injected into a subject through intradermal injection. In the present disclosure, the in vivo injection of the antigenic composition into the subject is followed by electroporation.

The antigenic composition of the present disclosure may further comprise an adjuvant. In the present disclosure, the adjuvant may be at least one of IL-7 and IL-33, preferably IL-33, but is not limited thereto.

According to another embodiment of the present disclosure, there is provided a vaccine comprising, as an active ingredient, any one or more recombinant peptides selected from the group consisting of:

a first recombinant peptide which comprises an amino acid sequence represented by SEQ ID NO: 287, or which is encoded by a first recombinant DNA comprising a nucleotide sequence represented by SEQ ID NO: 286:

a second recombinant peptide which comprises an amino acid sequence represented by SEQ ID NO: 289, or which is encoded by a second recombinant DNA comprising a nucleotide sequence represented by SEQ ID NO: 288:

a third recombinant peptide which comprises an amino acid sequence represented by SEQ ID NO: 291, or which is encoded by a third recombinant DNA comprising a nucleotide sequence represented by SEQ ID NO: 290:

a fourth recombinant peptide which comprises an amino acid sequence represented by SEQ ID NO: 293, or which is encoded by a fourth recombinant DNA comprising a nucleotide sequence represented by SEQ ID NO: 292; and

a fifth recombinant peptide which comprises an amino acid sequence represented by SEQ ID NO: 295, or which is encoded by a fifth recombinant DNA comprising a nucleotide sequence represented by SEQ ID NO: 294:

The vaccine of the present disclosure may comprise at least one of: the first recombinant peptide which comprises the amino acid sequence represented by SEQ ID NO: 287, or which is encoded by the first recombinant DNA comprising the nucleotide sequence represented by SEQ ID NO: 286; and the second recombinant peptide which comprises the amino acid sequence represented by SEQ ID NO: 289, or which is encoded by the second recombinant DNA comprising the nucleotide sequence represented by SEQ ID NO: 288.

The vaccine of the present disclosure may comprise at least one of: the first recombinant peptide which comprises the amino acid sequence represented by SEQ ID NO: 287, or which is encoded by the first recombinant DNA comprising the nucleotide sequence represented by SEQ ID NO: 286; and the second recombinant peptide which comprises the amino acid sequence represented by SEQ ID NO: 289, or which is encoded by the second recombinant DNA comprising the nucleotide sequence represented by SEQ ID NO: 288.

The vaccine of the present disclosure may comprise a peptide represented by the amino acid sequence of at least one of SEQ ID NOs: 1 to 76.

The vaccine of the present disclosure may comprise a peptide represented by the amino acid sequence of at least one of SEQ ID NOs: 1 to 38.

The vaccine of the present disclosure may comprise a peptide represented by the amino acid sequence of at least one of SEQ ID NOs: 39 to 76.

The vaccine of the present disclosure may comprise: a peptide represented by the amino acid sequence of at least one of SEQ ID NOs: 1 to 38; and a peptide represented by the amino acid sequence of at least one of SEQ ID NOs: 39 to 76.

The vaccine of the present disclosure may comprise a peptide represented by the amino acid sequence of at least one of SEQ ID NOs: 77 to 152.

The vaccine of the present disclosure may comprise a peptide represented by the amino acid sequence of at least one of SEQ ID NOs: 77 to 114.

The vaccine of the present disclosure may comprise a peptide represented by the amino acid sequence of at least one of SEQ ID NOs: 115 to 152.

The vaccine of the present disclosure may comprise: a peptide represented by the amino acid sequence of at least one of SEQ ID NOs: 77 to 114; and a peptide represented by the amino acid sequence of at least one of SEQ ID NOs: 115 to 152.

The vaccine of the present disclosure may comprise a peptide represented by the amino acid sequence of at least one of SEQ ID NOs: 153 to 186.

The vaccine of the present disclosure may comprise a peptide represented by the amino acid sequence of at least one of SEQ ID NOs: 187 to 227.

The vaccine of the present disclosure may comprise a peptide represented by the amino acid sequence of at least one of SEQ ID NOs: 187 to 207.

The vaccine of the present disclosure may comprise a peptide represented by the amino acid sequence of at least one of SEQ ID NOs: 208 to 227.

The vaccine of the present disclosure may comprise: a peptide represented by the amino acid sequence of at least one of SEQ ID NOs: 187 to 207; and a peptide represented by the amino acid sequence of at least one of SEQ ID NOs: 208 to 227.

The vaccine of the present disclosure may comprise a peptide represented by the amino acid sequence of at least one of SEQ ID NOs: 228 to 285.

The vaccine of the present disclosure may comprise a peptide represented by the amino acid sequence of at least one of SEQ ID NOs: 228 to 256.

The vaccine of the present disclosure may comprise a peptide represented by the amino acid sequence of at least one of SEQ ID NOs: 257 to 285.

The vaccine of the present disclosure may comprise: a peptide represented by the amino acid sequence of at least one of SEQ ID NOs: 228 to 256; and a peptide represented by the amino acid sequence of at least one of SEQ ID NOs: 257 to 285.

In the present disclosure, the vaccine is injected in vivo through a route selected from among intramuscular, intradermal, subcutaneous, subepidermal, transdermal and intravenous routes, but the route is not limited thereto. In the present disclosure, the vaccine is injected into a subject through intramuscular injection. In the present disclosure, the vaccine is injected into a subject through intradermal injection. In the present disclosure, the in vivo injection of the vaccine into the subject is followed by electroporation.

In the present disclosure, the vaccine may further comprise an adjuvant. In the present disclosure, the adjuvant may be at least one of IL-7 (SEQ ID NO: 296) and IL-33 (SEQ ID NO: 297), preferably IL-33, but is not limited thereto.

The antigenic composition or vaccine of the present disclosure may further comprise a solvent, an excipient, and the like. Examples of the solvent include, but are not limited, saline and distilled water, and examples of the excipient include, but are not limited to, aluminum phosphate, aluminum hydroxide, and aluminum potassium sulfate. In addition, the antigenic composition or vaccine of the present disclosure may further comprise substances that are commonly used for vaccine production in the art to which the present disclosure pertains.

The antigenic composition or vaccine of the present disclosure may be produced by methods that are commonly used in the art to which the present disclosure pertains. The antigenic composition or vaccine of the present disclosure may be prepared as an oral or parenteral formulation, and is preferably prepared as an injectable liquid formulation which is a parenteral formulation. The antigenic composition or vaccine of the present disclosure may be administered through an intradermal, intramuscular, intraperitoneal, intravenous, subcutaneous, nasal or epidural route.

The antigenic composition or vaccine of the present disclosure may be administered to a subject in an immunologically effective amount. The term “immunologically effective amount” refers to an amount sufficient to exhibit the effect of preventing or treating severe fever with thrombocytopenia syndrome (SFTS) or SFTS virus infection, and an amount that does not cause side effects or serious or excessive immune responses. The exact dose of the antigenic composition or vaccine of the present disclosure may vary depending on the specific immunogen to be administered, and may be easily determined by those skilled in the art depending on factors well known in the medical field, including the age, body weight, health and sex of a subject to be prevented or treated, the drug sensitivity of the subject, the route of administration, and the mode of administration. The antigenic composition or vaccine of the present disclosure may be administered once or several times.

The vaccine of the present disclosure is administered in a pharmaceutically effective amount. The term “pharmaceutically effective amount” refers to an amount sufficient to exhibit a vaccination effect, and an amount that does not cause side effects or serious or excessive immune responses. The exact dose of the vaccine may vary depending on the antigen to be administered, and may be easily determined by those skilled in the art depending on factors well known in the medical field, including the age, body weight, health and sex of a subject, the drug sensitivity of the subject, the route of administration, and the mode of administration. The vaccine may be administered once or several times.

The DNA vaccine of the present disclosure is a DNA vaccine in which a nucleotide encoding the epitope peptide of the present disclosure is contained in a pharmaceutically acceptable carrier. The DNA vaccine is preferably in the form of a DNA plasmid, most preferably in the form of a mock plasmid (derived from pVax-1), but is not limited thereto. Therefore, it is preferable that the above-described nucleotides are inserted into various known recombinant expression vectors.

According to still another embodiment of the present disclosure, there is provided an expression vector comprising any one or more recombinant DNAs selected from the group consisting of:

a first recombinant DNA comprising a nucleotide sequence represented by SEQ ID NO: 286;

a second recombinant DNA comprising a nucleotide sequence represented by SEQ ID NO: 288;

a third recombinant DNA comprising a nucleotide sequence represented by SEQ ID NO: 290:

a fourth recombinant DNA comprising a nucleotide sequence represented by SEQ ID NO: 292; and

a fifth recombinant DNA comprising a nucleotide sequence represented by SEQ ID NO: 294.

The expression vector of the present disclosure may comprise at least one recombinant DNA selected from among: the first recombinant DNA comprising the nucleotide sequence represented by SEQ ID NO: 286; and the second recombinant DNA comprising the nucleotide sequence represented by SEQ ID NO: 288.

The expression vector of the present disclosure may comprise: the first recombinant DNA comprising the nucleotide sequence represented by SEQ ID NO: 286: and the second recombinant DNA comprising the nucleotide sequence represented by SEQ ID NO: 288.

As used herein the term “vector” refers to a means for expressing a target gene in a host cell. The vector may comprise elements for expression of the target gene, including a replication origin, a promoter, an operator gene, and a terminator sequence, and may further comprise appropriate enzyme sites (e.g., restriction enzyme sites) for introduction into the genome of the host cell, and/or selection markers for confirming successful introduction into the host cell, and/or a ribosome binding site (RBS) for translation into a protein, internal ribosome entry site (IRES), and the like. The vector may be engineered by a conventional genetic engineering method so as to have a fusion polynucleotide (fusion promoter) as a promoter. The vector may further comprise a transcription control sequence (e.g., an enhancer, etc.), in addition to the promotor.

As used herein, the term “expression vector” refers to a recombinant vector capable of expressing a target peptide in a host cell of interest, and refers to a gene construct comprising essential regulatory elements operatively linked to express a gene insert. The expression vector comprises expression regulatory elements such as an initiation codon, a stop codon, a promoter and an operator. The initiation and stop codons are generally considered to be part of the nucleotide sequence encoding the polypeptide, and must exhibit an action in a subject when the gene construct has been administered and must be in frame with the coding sequence. The promoter of the vector may be constitutive or inducible. The vector may be introduced into a host cell in the form of an expression cassette, which is a gene construct including all elements necessary for self-expression. The expression cassette may include a promoter operatively linked to a gene insert to be expressed, a transcription termination signal, a ribosome binding site, and a translation termination signal. The expression cassette may be in the form of a self-replicable expression vector. In the present disclosure, the expression vector may be a viral or non-viral vector. The viral vector may be an adenovirus vector, a retrovirus vector including lentivirus, an adeno-associated virus vector, or a herpes simplex virus vector, but is not limited thereto. In addition, the non-viral vector may be a plasmid vector, a bacteriophage vector, a liposome, a bacterial artificial chromosome, or a yeast artificial chromosome, but is not limited thereto.

In the present disclosure, the target gene in the expression vector may be operatively linked to the fusion polynucleotide. The term “operatively linked” refers to a functional linkage between a gene expression regulatory sequence and another nucleotide sequence. The gene expression regulatory sequence may regulate transcription and/or translation of other nucleotide sequences by being “operatively linked”. In the expression vector, in order for the fusion polynucleotide to be operatively linked to the target gene, the fusion polynucleotide may be linked to the 5′ end of the target gene. The expression vector of the present disclosure may be used as a target protein expression vector capable of expressing the target protein with high efficiency in an appropriate host cell when the gene encoding the target protein to be expressed is operatively linked.

The expression vector of the present disclosure may further comprise, as an adjuvant, a gene encoding at least one of IL-7 (SEQ ID NO: 298) and IL-33 (SEQ ID NO: 299), preferably a gene encoding IL-33.

The expression vector of the present disclosure may further comprise a transcription regulatory sequence. The transcription regulatory sequence may be at least one selected from the group consisting of: a transcription termination sequence such as a polyadenylation sequence (pA); a replication origin such as an fl replication origin, an SV40 replication origin, a pMB1 replication origin, an adeno replication origin, an AAV replication origin, or a BBV replication origin; a Kozak sequence (AACAATGGC), which is known to be highly likely to increase gene expression by increasing the recognition rate of ribosomes at the initiation point (ATG) of the translation process; and an IgE leader sequence, but is not limited thereto.

The expression vector of the present disclosure may further comprise a restriction enzyme cleavage site. The restriction enzyme cleavage site refers to a specific nucleotide sequence that is specifically recognized and cleaved by a restriction enzyme. The cleavage sites may be sequences that are specifically recognized by restriction enzymes such as EcoRI, BamHI, HindIII, KpnI, SalI, NotI, NcoI, PstI, SmaI and XhoI.

In addition, the expression vector in the present disclosure may further include a selection marker. The selection marker is a gene for confirming whether the expression vector has been successfully introduced into a host cell or for constructing a stable cell line, and may be at least one selected from selected from the group consisting of, for example, genes resistant to drugs such as antibiotics, metabolism-related genes, and genes for gene amplification.

The expression vector of the present disclosure may include an IL-7-encoding gene (SEQ ID NO: 298) together with the recombinant DNA. In this case, the expression vector (preferably an expression plasmid) may comprise, as a restriction enzyme cleavage site, any one or more selected from the group consisting of BamHI, NcoI, and NotI. As an example, the expression vector may include a nucleotide sequence represented by SEQ ID NO: 300 together with the recombinant DNA.

The expression vector of the present disclosure may include an IL-33-encoding gene (SEQ ID NO: 299) together with the recombinant DNA. In this case, the expression vector (preferably an expression plasmid) may include, as a restriction enzyme cleavage site, any one or more selected from the group consisting of BamHI, NcoI and NotI. As an example, the expression vector may include a nucleotide sequence represented by SEQ ID NO: 301 together with the recombinant DNA.

In the present disclosure, the expression vector may be expressed in a host cell. For example, the host cell may strongly induce transcription initiation in animal cells. Specifically, the host cell may induce transcription initiation in mammalian cells, for example, animal cells such as human cells.

The expression vector of the present disclosure may be constructed by various methods known in the art.

According to yet another embodiment of the present disclosure, there is provided a transformant obtained by introducing the expression vector provided according to the present disclosure into a host cell by transformation.

In the present disclosure, transfer (introduction) of the expression vector into a cell may be performed using a transfer method well known in the art. Examples of the transfer method include, but are not limited to, microinjection, calcium phosphate precipitation, electroporation, sonoporation, magnetofection, liposome-mediated transfection, gene bombardment, and a method using dendrimers and inorganic nanoparticles.

The transfonant may be produced by transforming the above-described expression vector into a cell.

As used herein, the term “transformant” refers to a cell or plant transformed by a DNA construct comprising a DNA sequence which is operatively linked to a promoter and encodes a useful substance. In the present disclosure, the transformant is meant to include a transformed microorganism animal cell or plant cell, a transformed animal or plant, and a cultured cell derived therefrom.

According to still yet another embodiment of the present disclosure, there is provided a method for preventing or treating severe fever with thrombocytopenia syndrome (SFTS) virus infection, the method comprising a step of administering to a subject an effective amount of the above-described antigenic composition, the above-described vaccine, the above-described expression vector, or the above-described transformant.

According to a further embodiment of the present disclosure, there is provided a pharmaceutical composition for preventing or treating severe fever with thrombocytopenia syndrome (SFTS) virus infection, the pharmaceutical composition comprising, as an active ingredient, the above-described antigenic composition, the above-described vaccine, the above-described expression vector, or the above-described transformant.

In the present disclosure, the pharmaceutical composition may be in the form of capsules, tablets, granules, or injections, ointments, powders, or beverages, and the pharmaceutical composition may be for administration to humans.

For use, the pharmaceutical composition of the present disclosure may be formulated in the form of each of oral preparations, including powders, granules, capsules, tablets or aqueous suspensions, skin external preparations, suppositories, and sterile injectable solutions, according to conventional methods, but is not limited thereto. The pharmaceutical composition of the present disclosure may contain a pharmaceutically acceptable carrier. Pharmaceutically acceptable carriers that may be used in the present disclosure include: binders, lubricants, disintegrants, excipients, solubilizers, dispersing agents, stabilizers, suspending agents, colorants, fragrances and the like, which may be used for oral administration: buffers, preservatives, pain-relieving agents, solubilizers, isotonic agents, stabilizers and the like, which may be used for injection: and bases, excipients, lubricants, preservatives and the like, which may be used for local administration. The pharmaceutical composition of the present disclosure may be formulated in various ways by mixing it with the pharmaceutically acceptable carrier as described above. For example, for oral administration, the pharmaceutical composition of the present disclosure may be formulated in the form of tablets, troches, capsules, elixirs, suspensions, syrups, wafers or the like, and for injection, may be formulated in the form of unit dose ampoules or multi-dose vials. In addition, the pharmaceutical composition of the present disclosure may be formulated as solutions, suspensions, tablets, capsules, sustained-release preparations, or the like.

Meanwhile, examples of carriers, excipients and diluents suitable for formulation include lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinyl pyrrolidone, water, methylhydroxy benzoate, propylhydroxy benzoate, talc, magnesium stearate, and mineral oil. In addition, the pharmaceutical composition of the present disclosure may further contain a filler, an anticoagulant, a lubricant, a wetting agent, a fragrance, an emulsifier, a preservative or the like.

The routes of administration of the pharmaceutical composition according to the present disclosure include, but are not limited to, oral, intravenous, intramuscular, intra-arterial, intramedullary, intradural, intracardiac, transdermal, subcutaneous, intraperitoneal, intranasal, gastrointestinal, topical, sublingual and intrarectal routes. Oral or parenteral administration is preferred.

As used herein, the term “parenteral” is meant to include subcutaneous, transdermal, intravenous, intramuscular, intra-articular, intra-synovial, intrasternal, intradural, intra-lesional and intra-cranial injection or infusion techniques. The pharmaceutical composition of the present disclosure may also be formulated as suppositories for intrarectal administration.

The pharmaceutical composition of the present disclosure may vary depending on various factors, including the activity of a specific compound used, the patient's age, body weight, general health, sex and diet, the duration of administration, the route of administration, excretion rate, the drug content, and the severity of a specific disease to be prevented or treated. The dose of the pharmaceutical composition may vary depending on the patient's condition, body weight, the severity of the disease, the form of drug, and the route and period of administration, but may be suitably selected by a person skilled in the art and may be 0.0001 to 50 mg/kg/day or 0.001 to 50 mg/kg/day. The pharmaceutical composition may be administered once or several times a day. The dose is not intended to limit the scope of the present disclosure in any way. The pharmaceutical composition according to the present disclosure may be formulated as pills, sugar-coated tablets, capsules, liquids, gels, syrups, slurries, or suspensions.

### Advantageous Effects

A recombinant DNA or peptide of SFTS virus and an SFTS virus vaccine comprising the same, which are provided according to the present disclosure, exhibit an excellent effect on the prevention and treatment of SFTS virus infection by effectively inducing an immune response to SFTS virus in a subject.

### BRIEF DESCRIPTION OF DRAWINGS

FIG. 1 is a well image showing the results of ELISpot analysis performed to measure T-cell response specific to SFTS virus.

FIGS. 2A to 2E are graphs showing T-cell immune responses to SFTSV vaccine candidates.

FIGS. 3A to 3C are graphs showing the results of evaluating the multifunctionality of T cells by SFTSV DNA vaccine candidates.

FIG. 4 is a graph showing the multifuctionality of T-cells induced by vaccine candidates.

FIGS. 5A to 5F are graphs showing SFTSV-specific antibody production induced by vaccines.

FIG. 6 is a graph showing the results of quantitatively evaluating neutralizing antibody titers induced by SFTSV DNA vaccines.

FIG. 7 is a graph showing the results of validating the infection inhibitory effect of an SFTSV vaccine.

FIGS. 8A to 8D are graphs showing SFTSV-specific T cell immune responses induced by SFTSV vaccine candidates in medium-sized animals.

FIG. 9 is a graph showing the results of measuring, using ELISA assay, the formation of an SFTSV-specific reactive antibody formed by a DNA vaccine.

FIG. 10 is a graph showing the results of measuring, using PRNT50 assay, the neutralizing antibody titer of an antibody induced by a DNA vaccine.

FIG. 11 is a graph showing the survival rate in SFTSV-infected medium-sized animal models.

FIGS. 12A to 12C are graphs showing the results of measuring SFTSV virus load by real-time PCR.

FIGS. 13A to 13C are graphs showing the results of counting platelets.

FIGS. 14A and 14B are graphs showing the results of measuring body weight and body temperature after SFTSV infection.

FIGS. 15A and 15B are graphs showing the results of a PRNT50 test performed to evaluate the cross-reactivity of an SFTSV neutralizing antibody formed in mice after administration of SFTSV DNA vaccines.

FIG. 16 is a graph showing the results of identifying T-cell immune responses induced by vaccines in SFTSV-infected medium-sized models.

FIG. 17 is a graph showing the results of quantitatively evaluating antibody immune responses and neutralizing antibody titers induced by vaccines in SFTSV-infected medium-sized animal models.

FIG. 18 is a graph showing the results of evaluating the preventive effects of SFTSV-preventive DNA vaccines in SFTSV-infected medium-sized animal models.

FIG. 19 depicts graphs showing the results of measuring SFTSV virus load by real-time PCR.

FIG. 20 is a graph showing the results of measuring SFTSV virus load by real-time PCR

FIG. 21 depicts graphs showing the results of counting platelets after SFTSV infection.

FIG. 22 is a graph showing the results of counting platelets after SFTSV infection.

FIG. 23 depicts graphs showing the results of counting white blood cells.

FIG. 24 is a graph showing the results of counting white blood cells.

FIG. 25 depicts graphs showing the body weights of control animals after SFTSV infection.

FIG. 26 is a graph showing the body weights of control animals after SFTSV infection.

FIG. 27 depicts graphs showing the body temperatures of control animals after SFTSV infection.

FIG. 28 is a graph showing the body temperatures of control animals after SFTSV infection.

FIG. 29 depicts graphs showing serum ALT concentrations.

FIG. 30 is a graph showing serum ALT concentrations.

FIG. 31 depicts graphs showing serum AST concentrations.

FIG. 32 is a graph showing serum AST concentrations.

FIG. 33 is a view showing an SFTSV Gc expression plasmid (pGX-SFTSV Gc_hCO, 4635 bp).

FIG. 34 is a view showing an SFTSV Gn expression plasmid (pGX-SFTSV Gn_hCO, 4626 bp).

FIG. 35 is a view showing an SFTSV NP expression plasmid (pGX-SFTSV NP_hCO, 3756 bp).

FIG. 36 is a view showing an SFTSV NS expression plasmid (pGX-SFTSV NS_hCO, 3900 bp).

FIG. 37 is a view showing an SFTSV RdRp expression plasmid (pGX-SFTSV RdRp_hCO, 9273 bp).

FIG. 38 is a view showing a mouse IL-7 expression plasmid (pGX-mIL-7_mCO, 3483 bp).

FIG. 39 is a view showing an IL-33 expression plasmid (pGX-mIL-33_mCO, 3819 bp).

### BEST MODE

According to one embodiment of the present disclosure, there is provided an antigenic composition or vaccine comprising, as an active ingredient, any one or more recombinant peptides selected from the group consisting of: a first recombinant peptide which comprises an amino acid sequence represented by SEQ ID NO: 287, or which is encoded by a first recombinant DNA comprising a nucleotide sequence represented by SEQ ID NO: 286; a second recombinant peptide which comprises an amino acid sequence represented by SEQ ID NO: 289, or which is encoded by a second recombinant DNA comprising a nucleotide sequence represented by SEQ ID NO: 288; a third recombinant peptide which comprises an amino acid sequence represented by SEQ ID NO: 291, or which is encoded by a third recombinant DNA comprising a nucleotide sequence represented by SEQ ID NO: 290; a fourth recombinant peptide which comprises an amino acid sequence represented by SEQ ID NO: 293, or which is encoded by a fourth recombinant DNA comprising a nucleotide sequence represented by SEQ ID NO: 292: and a fifth recombinant peptide which comprises an amino acid sequence represented by SEQ ID NO: 295, or which is encoded by a fifth recombinant DNA comprising a nucleotide sequence represented by SEQ ID NO: 294.

The antigenic composition or vaccine of the present disclosure may further comprise an adjuvant. In this case, the adjuvant may be at least one of IL-7 and IL-33. preferably IL-33, but is not limited thereto.

Mode for Invention Hereinafter, the present disclosure will be described in detail with reference to examples. However, the following examples serve merely to illustrate the present disclosure, and the scope of the present disclosure is not limited by the following examples.

Synthesis of Five SFTS Virus Antigen Genes and Two Adjuvant Genes

IgE leader and Kozak sequences were inserted into the 5′ end of each target gene (SFTSV antigen or adjuvant gene), and a termination codon was inserted into the 3′ end of the target gene. Finally, restriction enzyme sequences (5′ BamHI and 3′ Not) were inserted into both ends of the gene, followed by gene synthesis.

Cloning into High-Efficiency Backbone Plasmid (pGX0001)

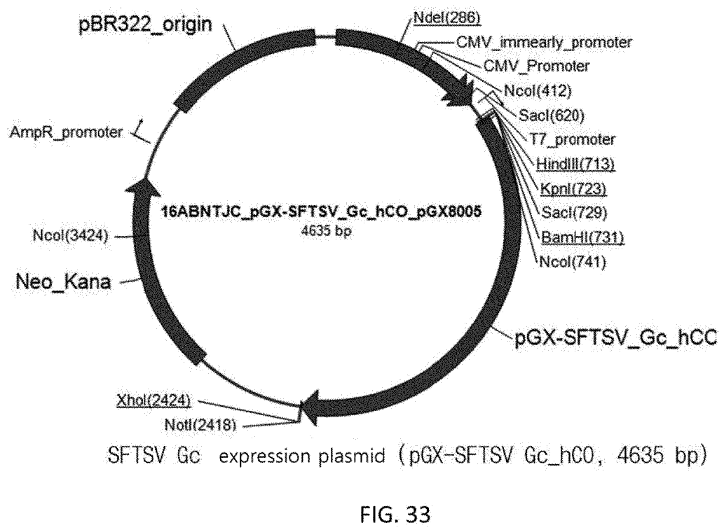

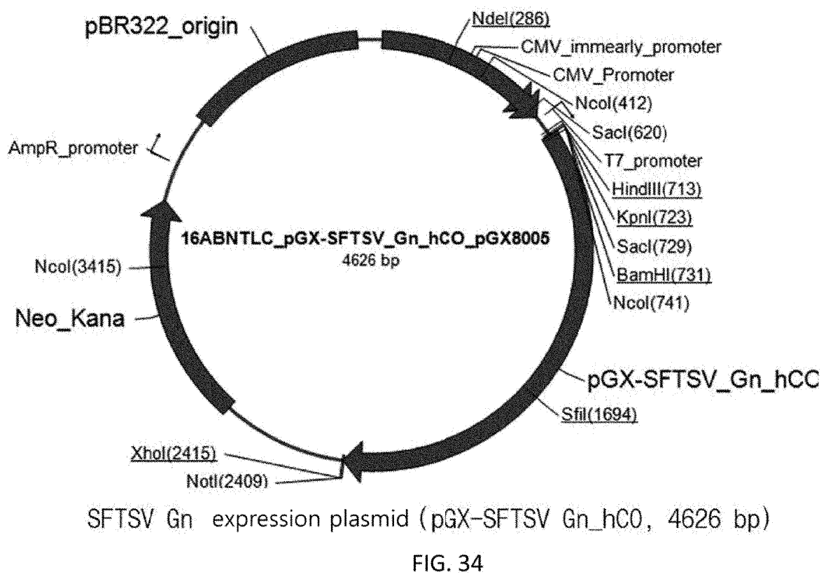

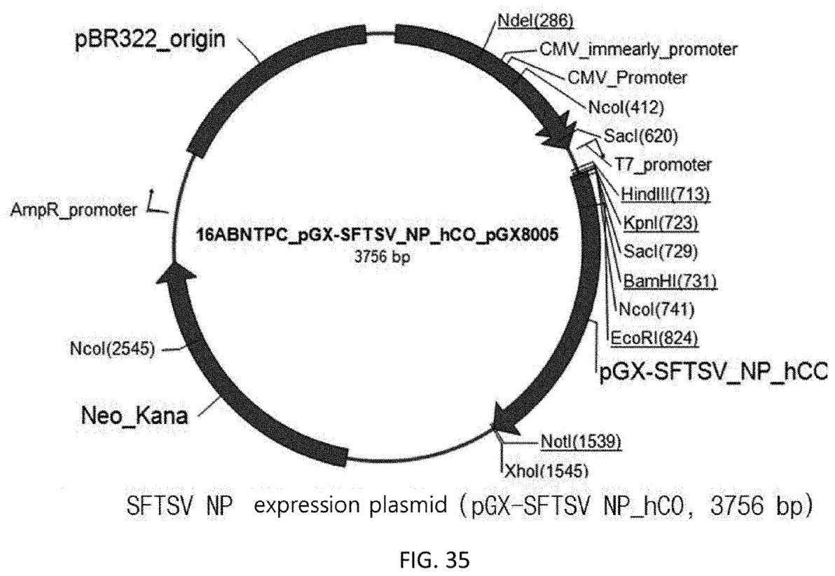

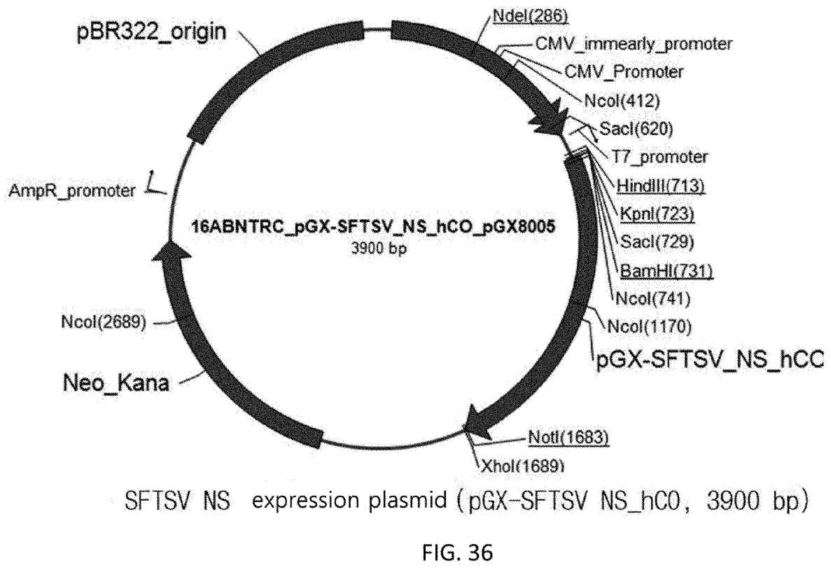

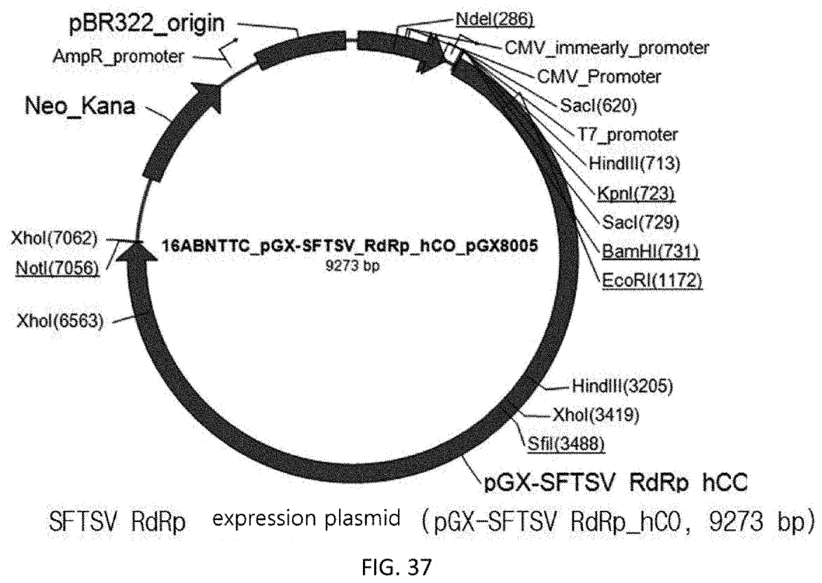

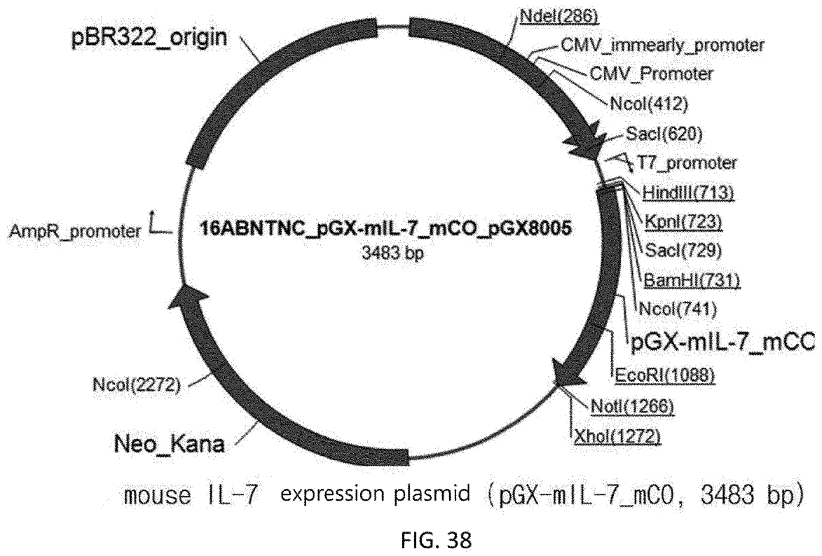

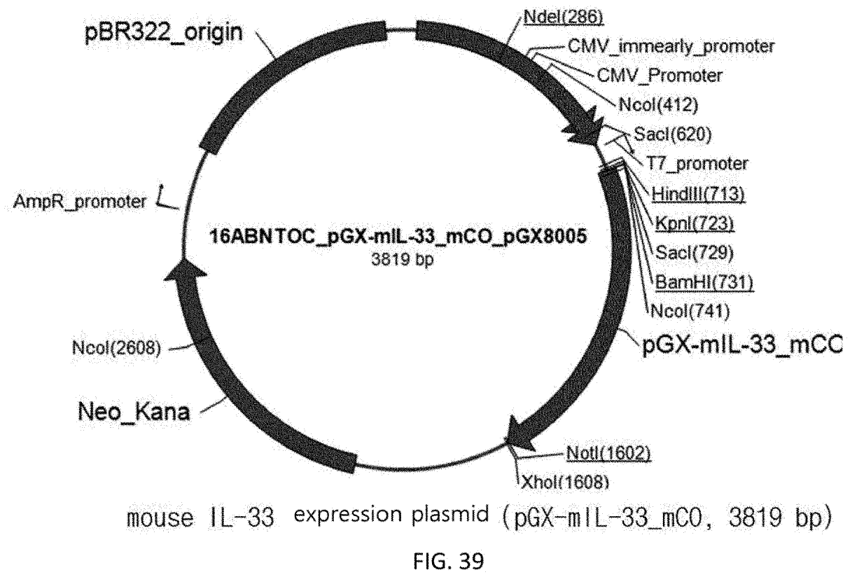

After completion of synthesis of each insertion gene, each insertion gene was cleaved with BamHI and NotI and inserted into a high-efficiency backbone plasmid (pGX0001) cleaved with the same restriction enzymes, thereby constructing candidate plasmids (see FIGS. 33 to 39). The results of gene synthesis and cloning were confirmed by nucleotide sequencing.

Optimization of Sequence of DNA Vaccine Highly Expressing Antigen In Vivo

As five optimal antigens. Gc (glycoprotein C). Gn (glycoprotein N). NP (nucleocapsid protein), NS (non-structural protein) and RdRp (RNA dependent RNA polymerase) were selected, and as two optimal adjuvants, IL-7 and IL-33 were selected. For the five antigens, a consensus sequence derived from 27 to 32 SFTS virus strains isolated from Korean. Chinese and Japanese was secured. The consensus sequence was designed as a universal antigen sequence with cross-immunity using the amino acid sequence of an antigen common to various SFTS virus subtypes and variants. Major human MHC class I and II epitopes present in various SFTS virus subtypes and variants were identified by using in silico immunoinformatics techniques, and antigen sequences were designed to contain these epitopes. Thereafter, based on the optimized amino acid sequences of the SFTS virus antigens, nucleotide sequences for DNA vaccines were finally derived.

Expression cassette structures according to one embodiment of the present disclosure, which are used in the following experiment, were configured to contain a high-expression promoter (plasmid backbone sequence), a Kozak sequence, an IgE leader sequence, and a poly-A signal sequence (plasmid backbone sequence). At this time, the Kozak and IgE leader sequences were inserted upstream of the target gene (SFTSV antigen or adjuvant) in order to increase the expression level of the gene in vivo. Meanwhile, in order to increase the expression level of the antigen gene in vivo, the sequences of five SFTS virus antigens (Gc, Gn, NP, NS, and RdRp) were optimized with human codons.

Generation of DNA Vaccine Lead Candidates Expressing SFTS Virus Antigen

An IgE leader sequence and a Kozak sequence were inserted into 5′ end of each target gene (SFTSV antigen or adjuvant gene), and a stop codon was inserted into the 3′ end thereof. Finally, restriction enzyme sequences (5′ BamHI and 3′ NotI) were inserted into both ends of each gene, followed by gene synthesis. After completion of synthesis of each insertion gene, each insertion gene was cleaved with BamHI and NotI and inserted into a high-efficiency backbone plasmid (pGX0001) cleaved with the same restriction enzymes, thereby constructing candidate plasmids. The results of gene synthesis and cloning were confirmed by gene sequencing.

Evaluation of Immunogenicity of SFTS Virus Antigen-Expressing DNA Vaccines Using Mouse Models

Overlapping peptide (OLP) pools for immunogenicity evaluation were created.

Specifically, in order to evaluate the immunogenicity of the five SFTS virus antigens, the sequence of each antigen was fragmented into 15-mer peptides overlapping 8 amino acids. The purity of each of the peptides was qualitatively and quantitatively analyzed using high performance liquid chromatography and electrospray mass spectrometry in the production process. Through this process, a total of 76 peptides were obtained from the Gn antigen, and 38 of these peptides were mixed together to prepare OLP1 and OLP2 (25 μg/ml each peptide) for Gn (Table 1). From the Gc antigen, a total of 76 peptides were obtained and 38 of these peptides were mixed together to prepare OLP3 and OLP4 (Table 2). From the NP antigen, a total of 34 peptides were obtained and mixed together to prepare OLP5 (Table 3). From the NS antigen, a total of 41 peptides were obtained and mixed together to prepare OLP6 (Table 4). From the RdRp antigen, a total of 58 peptides were obtained, and 29 of these peptides mixed together to prepare OLP7 and OLP8 (Table 5).

More specifically, as shown in Tables 1 to 5 below, the mixture of SEQ ID NO: 1 to SEQ ID NO: 38 is OLP1. In addition, the mixture of SEQ ID NO: 39 to SEQ ID NO: 76 is OLP2. The mixture of SEQ ID NO: 77 to SEQ ID NO: 114 is OLP3. The mixture of SEQ ID NO: 115 to SEQ ID NO: 152 is OLP4. The mixture of SEQ ID NO: 153 to SEQ ID NO: 186 is OLP5. The mixture of SEQ ID NO: 187 to SEQ ID NO: 227 is OLP6. The mixture of SEQ ID NO: 228 to SEQ ID NO: 256 is OLP7. The mixture of SEQ ID NO: 257 to SEQ ID NO: 285 is OLP8.

The OLP pools created as described above were used for evaluation of T-cell immune responses in the following experiment.

TABLE 1
SFTSV consensus glycoprotein Gn sequence
Gn (535 aa)
MMKVIWFSSLICLVIQCSGDTGPIICAGPIHSNKSANIPHLLGYSEKI
CQIDRLIHVSSWLRNHSQFQGYVGQRGGRSQVSYYPAENSYSRWSGLL
SPCDADWLGMLVVKKAKGSDMIVPGPSYKGKVFFERPTFDGYVGWGCG
SGKSRTESGELCSSDSGTSSGLLPSDRVLWIGDVACQPMTPIPEETFL
ELKSFSQSEFPDICKIDGIVFNQCEGESLPQPFDVAWMDVGHSHKIIM
REHKTKWVQESSSKDFVCYKEGTGPCSESEEKTCKTSGSCRGDMQFCK
VAGCEHGEEASEAKCRCSLVHKPGEVVVSYGGMRVRPKCYGFSRMMAT
LEVNPPEQRIGQCTGCHLECINGGVRLITLTSELKSATVCASHFCSSA
TSGKKSTEIQFHSGSLVGKTAIHVKGALVDGTEFTFEGGSCMFPDGCD
AVDCTFCREFLKNPQCYPAKKWLFIIIVILLGYAGLMLLTNVLKAIGV
WGSWVIAPVKLMFAIIKKLMRSVSCLMGKLMDRGRQVIHEEIGENREG
NQDDVRIE* (SEQ ID NO: 298)

| SEQ | | SEQ | |
|---|---|---|---|
| ID NO | sequence | ID NO | sequence |
| 1 | MMKVIWFSSLICLVI | 39 | SESEEKTCKTSGSCR |
| 2 | SSLICLVIQCSGDTG | 40 | CKTSGSCRGDMQFCK |
| 3 | IQCSGDTGPIICAGP | 41 | RGDMQFCKVAGCEHG |
| 4 | GPIICAGPIHSNKSA | 42 | KVAGCEHGEEASEAK |
| 5 | PIHSNKSANIPHLLG | 43 | GEEASEAKCRCSLVH |
| 6 | ANIPHLLGYSEKICQ | 44 | KCRCSLVHKPGEVVV |
| 7 | GYSEKICQIDRLIHV | 45 | HKPGEVVVSYGGMRV |
| 8 | QIDRLIHVSSWLRNH | 46 | VSYGGMRVRPKCYGF |
| 9 | VSSWLRNHSQFQGYV | 47 | VRPKCYGFSRMMATL |
| 10 | HSQFQGYVGQRGGRS | 48 | FSRMMATLEVNPPEQ |
| 11 | VGQRGGRSWVSYYPA | 49 | LEVNPPEQRIGQCTG |
| 12 | SQVSYYPAENSYSRW | 50 | QRIGQCTGCHLECIN |
| 13 | AENSYSRWSGLLSPC | 51 | GCHLECINGGVRLIT |
| 14 | WSGLLSPCDADWLGM | 52 | NGGVRLITLTSELKS |
| 15 | CDADWLGMLVVKKAK | 53 | TLTSELKSATVCASH |
| 16 | MLVVKKAKGSDMIVP | 54 | SATVCASHGCSSATS |
| 17 | KGSDMIVPGPSYKGK | 55 | HFCSSATSGKKSTEI |
| 18 | PGPSYKGKVFFERPT | 56 | SGKKSTEIQFHSGSL |
| 19 | KVFFERPTFDGYVGW | 57 | IQFHSGSLVGKTAIH |
| 20 | TFDGYVGWGCGSGKS | 58 | LVGKTAIHVKGALVD |
| 21 | WGCGSGKSRTESGEL | 59 | HVKGALVDGTEFTFE |
| 22 | SRTESGELCSSDSGT | 60 | DGTEFTFEGSCMFPD |
| 23 | LCSSDSGTSSGLLPS | 61 | EGSCMFPDGCDAVDC |
| 24 | TSSGLLPSDRVLWIG | 62 | DGCDAVDCTFCREFL |
| 25 | SDRVLWIGDVACQPM | 63 | CTFCREFLKNPQCYP |
| 26 | GDVACQPMTPIPEET | 64 | LKNPQCYPAKKWLFI |
| 27 | MTPIPEETFLELKSF | 65 | PAKKWLFIIIVILLG |
| 28 | TFLELKSFSQSEFPD | 66 | IIIVILLGYAGLMLL |
| 29 | FSQSEFPDICKIDGI | 67 | GYAGLMLLTNVLKAI |
| 30 | DICKIDGIVFNQCEG | 68 | LTNVLKAIGVWGSWV |
| 31 | IVFNQCEGESLPQPF | 69 | IGVWGSWVIAPVKLM |
| 32 | GESLPQPFDVAWMDV | 70 | VIAPVKLMFAIIKKL |
| 33 | FDVAWMDVGHSHKII | 71 | MFAIIKKLMRSVSCL |
| 34 | VGHSHKIIMREHKTK | 72 | LMRSVSCLMGKLMDR |
| 35 | IMREHKTKWVQESSS | 73 | LMGKLMDRGRQVIHE |
| 36 | KWVQESSSKDFVCYK | 74 | RGRQVIHEEIGENRE |
| 37 | SKDFVCYKEGTGPCS | 75 | EEIGENREGNQDDVR |
| 38 | KEGTGPCSESEEKTC | 76 | EGNQDDVRIE |

TABLE 2
SFTSV consensus glycoprotein Gc sequence
Gc (538 aa)
MARPRRVRHWMYSPVILTILAIGLAEGCDEMVHADSKLVSCRQGSGNM
KECVTTGRALLPAVNPGQEACLHFTAPGSPDSKCLKIKVKRINLKCKK
SSSYFPVPDARSRCTSVRRCRWAGDCQSGCPPHFTSNSFSDDWAGKMD
RAGLGFSGCSDGCGGAACGCFNAAPSCIFWRKWVENPHGIIWKVSPCA
AWVPSAVIELTMPSGEVRTFHPMSGIPTQVFKGVSVTYLGSDMEVSGL
TDLCEIEELKSKKLALAPCNQAGMGVVGKVGEIQCSSEESARTIKKDG
CIWNADLVGIELRVDDAVCYSKITSVEAVANYSAIPTTIGGLRFERSH
DSQGKISGSPLDITAIRGSFSVNYRGLRLSLSEITATCTGEVTNVSGC
YSCMTGAKVSIKLHSSKNSTAHVRCKGDETAFSVLEGVHSYTVSLSFD
HAVVDEQCQLNCGGESQVTLKGNLIFLDVPKFVDGSYMQTYHSTVPTG
ANIPSPTDWLNALFGNGLSRWILGVIGVLLGGLALFFLIMSLFKLGTK
QVFRSRTKLA* (SEQ ID NO: 289)

| SEQ | | SEQ | |
|---|---|---|---|
| ID NO | sequence | ID NO | sequence |
| 77 | MARPRRVRHWMYSPV | 115 | GKVGEIQCSSEESAR |
| 78 | RHWMYSPVILTILAI | 116 | CSSEESARTIKKDGC |
| 79 | VILTILAIGLAEGCD | 117 | RTIKKDGCIWNADLV |
| 80 | IGLAEGCDEMVHADS | 118 | CIWNADLVGIELRVD |
| 81 | DEMVHADSKLVSCRQ | 119 | VGIELRVDDAVCYSK |
| 82 | SKLVSCRQGSGNMKE | 120 | DDAVCYSKITSVEAV |
| 83 | QGSGNMKECVTTGRA | 121 | KITSVEAVANYSAIP |
| 84 | ECVTTGRALLPAVNP | 122 | VANYSAIPTTIGGLR |
| 85 | ALLPAVNPGQEACLH | 123 | PTTIGGLRFERSHDS |
| 86 | PGQEACLHFTAPGSP | 124 | RFERSHDSQGKISGS |
| 87 | HFTAPGSPDSKCLKI | 125 | SQGKISGSPLDITAI |
| 88 | PDSKCLKIKVKRINL | 126 | SPLDITAIRGSFSVN |
| 89 | IKVKRINLKCKKSSS | 127 | IRGSFSVNYRGLRLS |
| 90 | LKCKKSSSYFVPDAR | 128 | NYRGLRLSLSEITAT |
| 91 | SYFVPDARSRCTSVR | 129 | SLSEITATCTGEVTN |
| 92 | RSRCTSVRRCRWAGD | 130 | TCTGEVTNVSGCYSC |
| 93 | RRCRWAGDCQSGCPP | 131 | NVSGCYSCMTGAKVS |
| 94 | DCQSGCPPHFTSNSF | 132 | CMTGAKVSIKLHSSK |
| 95 | PHFTSNSFSDDWAGK | 133 | SIKLHSSKNSTAHVR |
| 96 | FSDDWAGKMDRAGLG | 134 | KNSTAHVRCKGDETA |
| 97 | KMDRAGLGFSGCSDG | 135 | RCKGDETAFSVLEGV |
| 98 | GFSGCSDGCGGAACG | 136 | AFSVLEGVHSYTVSL |
| 99 | GCGGAACGCFNAAPS | 137 | VHSYTVSLSFDHAVV |
| 100 | GCFNAAPSCIFWRKW | 138 | LSFDHAVVDEQCQLN |
| 101 | SCIFWRKWVENPHGI | 139 | VDEQCQLNCGGHESQ |
| 102 | WVENPHGIIWKVSPC | 140 | NCGGHESQVTLKGNL |
| 103 | IIWKVSPCAAWVPSA | 141 | QVTLKGNLIFLDVPK |
| 104 | CAAWVPSAVIELTMP | 142 | LIFLDVPKFVDGSYM |
| 105 | AVIELTMPSGEVRTF | 143 | KFVDGSYMQTYHSTV |
| 106 | PSGEVRTFHPMSGIP | 144 | MQTYHSTVPTGANIP |
| 107 | FHPMSGIPTQVFKGV | 145 | VPTGANIPSPTDWLN |
| 108 | PTQVFKGVSVTYLGS | 146 | PSPTDWLNALGFNGL |
| 109 | VSVTYLGSDMEVSGL | 147 | NALFGNGLSRWILGV |
| 110 | SDMEVSGLTDLCEIE | 148 | LSRWILGVIGVLLGG |
| 111 | LTDLCEIEELKSKKL | 149 | VIGVLLGGLALFFLI |
| 112 | EELKSKKLALAPCNQ | 150 | GLALFFLIMSLFKLG |
| 113 | LALAPCNQAGMGVVG | 151 | IMSLFKLGTKQVFRS |
| 114 | QAGMGVVGKVGEIQC | 152 | GTKQVFRSRTKLA |

TABLE 3
SFTSV consensus nuclear protein NP sequence
NP (245 aa)
MSEWSRIAVEFGEQQLNLTELEDFARELAVEGLDPALIIKKLKETGG
WVKDTKFIIVFALTRGNKIVKASGKMSNSGSKRLMALQEKYGLVERA
ETRLSITPVRVAQSLPTWTCAAAAALKEYLPVGPAVMNLKVENYPPE
MMCMAFGSLIPTAGVSEATTKTLMEAYSLWQDAFTKTINVKMRGASK
TEVYNSFRDPLHAAVNSVFFPNDVRVKWLKAGKILGPDGVPSRAAEV
AAAAYRNL* (SEQ ID NO: 291)

| SEQ ID NO | sequence |
|---|---|
| 153 | MSEWSRIAVEFGEQQ |
| 154 | AVEFGQEELNLTLEL |
| 155 | QLNLTELEDFARELA |
| 156 | EDFARELAYEGLDPA |
| 157 | AYEGLDPALIIKKLK |
| 158 | ALIIKKLKETGGDDW |
| 159 | KETGGDDWVKDTKFI |
| 160 | WVKDTKFIIVFALTR |
| 161 | IIVFALTRGNKIVKA |
| 162 | RGNKIVKASGKMSNS |
| 163 | ASGKMSNSGSKRLMA |
| 164 | SGSKRLMALQEKYGL |
| 165 | ALQEKYGLVERAETR |
| 166 | LVERAETRLSITPVR |
| 167 | RLSITPVRVAQSLPT |
| 168 | RVAQSLPTWTCAAAA |
| 169 | TWTCAAAAALKEYLP |
| 170 | AALKEYLPVGPAVMN |
| 171 | PVGPAVMNLKVENYP |
| 172 | NLKVENYPPEMMCMA |
| 173 | PPEMMCMAFGSLIPT |
| 174 | AFGSLIPTAGVSEAT |
| 175 | TAGVSEATTKTLMEA |
| 176 | TTKTLMEAYSLWQDA |
| 177 | AYSLWQDAFTKTINV |
| 178 | AFTKTINVKMRGASK |
| 179 | VKMRGASKTEVYNSF |
| 180 | KTEVYNSFRDPLHAA |
| 181 | FRDPLHAAVNSVFFP |
| 182 | AVNSVFFPNDVRVKW |
| 183 | PNDVRVKWLKAKGIL |
| 184 | WLKAKGILGPDGPPS |
| 185 | LGPDGVPSRAAEVAA |
| 186 | SRAAEVAAAAYRNL |

TABLE 4
SFTSV consensus non-structural protein NS
sequence
NS (293 aa)
MSLSKCSNVDLKSVAMNANTVRLEPSLGEYPTLRRDLVECSCSVLTLS
MVKRMGKMTNTVWLFGNPKNPLHQLEPGLEQLLDMYYKDMRCYSQREL
SALRWPSGKPSVWFLQAAHMFFSIKNSWAMETGRENWRGLFHRITKGQ
KYLFEGDMILDSLEAIEKRRLRGLPEILITGLSPILDVAAQIESLARL
GMSLNHHLFTSSSLRKPLLDCWDFFIPIRKKKTDGSYSVLDEDDEPGV
LQGYPYLMAHYLNRCPFHNLIRFDEELRTAALNTIWGRDWPAIGDLPK
EV* (SEQ ID NO: 239)

| SEQ | | SEQ | |
|---|---|---|---|
| ID NO | sequence | ID NO | sequence |
| 187 | MSLSKCSNVDLKSVA | 208 | FEGDMILDSLEAIEK |
| 188 | NVDLKSVAMNANTVR | 209 | DSLEAIEKRRLRLGL |
| 189 | AMNANTVRLEPSLGE | 210 | KRRLRLGLPEILITG |
| 190 | RLEPSLGEYPTLRRD | 211 | LPEILITGLSPILDV |
| 191 | EYPTLRRDLVECSCS | 212 | GLSPILDVALLQIES |
| 192 | DLVECSCSVLTLSMV | 213 | VALLQIESLARLRGM |
| 193 | SVLTLSMVKRMGKMT | 214 | SLARLRGMSLNHHLF |
| 194 | VKRMGKMTNTVWLFG | 215 | MSLNHHLFTSSSLRK |
| 195 | TNTVWLFGNPKNPLH | 216 | FTSSSLRKPLLDCWD |
| 196 | GNPKNPLHQLEPGLE | 217 | KPLLDCWDFFIPIRK |
| 197 | HQLEPGLEQLLDMYY | 218 | DFFIPIRKKKTDGSY |
| 198 | EQLLDMYYKDMRCYS | 219 | KKKTDGSYSVLDEDD |
| 199 | YKDMRCYSQRELSAL | 220 | YSVLDEDDEPGVLQG |
| 200 | SQRELSALRWPSGKP | 221 | DEPGVLQGYPYLMAH |
| 201 | LRWPSGKPSVWFLQA | 222 | GYPYLMAHYLNRCPF |
| 202 | PSVWFLQAAHMFFSI | 223 | HYLNRCPFHNLIRFD |
| 203 | AAHMFFSIKNSWAME | 224 | FHNLIRFDEELRTAA |
| 204 | IKNSWAMETGRENWR | 225 | DEELRTAALNTIWGR |
| 205 | ETGRENWRGLFHRIT | 226 | ALNTIWGRDWPAIGD |
| 206 | RGLFHRITKGQKYLF | 227 | RDWPAIGDLPKEV |
| 207 | TKGQKYLFEGDMILD | | |

TABLE 5
SFTSV consensus RNA dependent RNA
polymerase RdRp sequence
RdRp (2085 aa)
MNLEVLCGRINVENGLSLGEPGLYDQIYDRPGLPDLDVTVDATGVTVD
IGAVPDSASQLGSSINAGLITIQLSEAYKINHDFTFSGLSKTTDRRLS
EVFPITHDGSDGMTPDVIHTRLDGTIVVVEFSTTRSHNIGGLEAAYRT
KIEKVYRDPISRRVDIMENPRVFFGVIVVSSGGVLSNMPLTQDEAEEL
MYRFCIANEIYTKARSMDADIELQKSEEELEAISRALSFFSLFEPNIE
RVEGTFPNSEIEMLEQFLSTPADVDFITKTLKAKEVEAYADLCDSHYL
KPEKTIQERLEINRCEAIDKTQDLLALHARSNKQTSLNRGTVKLPPWL
PKPSSESIDIKTDSGFGSLMDHGAYGELWAKCLLDVSLGNVEGVVSDP
AKELDIAISDDPEKDTPKEAKITYRRFKPALSSSARQEFSLQGVEGKK
WKRMAANQKKESESHETLSPFLDVEIDIGFLTFNNLLADSRYGDESVQ
RAVSILLEAKASAMQDTELTHALNDSFKRNLSSNVVQWSLWVSCLAQE
LASALKQHCRAGEFIIKKLKFWPIYVIIKPTKSSSHIFYSLGIRKADV
TRRLTGRVFSDTIDAGEWELTEFKSLKTCKLTNLVNLPCTMLNSIAFW
REKLGVAPWLVRKPCSELREQVGLTFLISLEDKSKTEEIITLTRYTQM
EGFVSPPMLPKPQKMLGKLDGPLRTKLQVYLLRKHLDCMVRIASQPFS
LIPREGRVEWGGTFHAISGRSTNLENMVNSWYIGYYKNKEESTELNAL
GEMYKKIVEMEEDKPSSPEFLGWGDTDSPKKHEFSRSFLRAACSSLER
EIAQRHGRQWKQNLEERVLREIGTKNILDLASMKATSNFSKDWELYSE
VQTKEYHRSKLLEKMATLIEKGVMWYIDAVGQAWKAVLDDGCMRICLF
KKNQHGGRLEIYVMDANARLVQFGVETMARCVCELSPHETVANPRLKN
SIIENHGLKSARSLGPGSININSSNDAKKWNQGHYTTKLALVLCWFMP
AKFHRFIWAAISMFRRKKMMVDLRFLAHLSSKSESRSSDPFREAMTDA
FHGNREVSWMDKGRTYIKTETGMMQGLILHFTSSLLHSCVQSFYKSYF
VSKLKEGYMGESISGVVDVIEGSDDSAIMISTRPKSDMDEVRSRFFVA
NLLHSVKFLNPLFGIYSSEKSTVNTVYCVEYNSEFHFHRHLVRPTLRW
IAASHQISETEALASRQEDYSNLLTQCLEGGASFSLTYLIQCAQLLHH
YMLLGLCLHPLFGTFMGMLISDPDPALGFFLMDNPAFAGGAGFRFNLW
RACKTTDLGRKYAYYFNEIQGKTKGDEDYRALDATSGGTLSHSVMVYW
GDRKKYQALLNRMGLPEDWVEQIDENPGVLYRRAANKKELLLKLAEKV
HSPGVTSSLSKGHVVPRVVAAGVYLLSRHCFRFSSSIHGRGSAQKASL
IKLLMNSSISAMKHGGSLNPNQERMLFPQAQEYDRVCTLLEEVEHLTG
KFVVRERNIVRSRIDLFQEPVDLRCKAEDLVSEVWFGLKRTKLGPRLL
KEEWDKLRASFAWLSTDPSETLRDGPFLSHVQFRNFIAHVDAKSRSVR
LLGAPVKKSGGVTTISQVVRMNFFPGFSLEAEKSLDNQERLESISILK
HVLFMVLNGPYTEEYKLEMIIEAFSTLVIPQPSEVIRKSRTMTLCLLS
NYLSSRGGSILDQIERAQSGTLGGFSKPQKTFIRPGGGIGYKGKGVWT
GVMEDTHVQILIDGDGTSNWLEEIRLSSDARLYDVIESIRRLCDDLGI
NNRVASAYRGHCMVRLSGFKIKPASRTDGCPVRIMERGFRIRELQNPD
EVKMRVRGDILNLSTIQEGRVMNILSYRPRDTDISESAAAYLWSNRDL
FSFGKKEPSCSWICLKTLDNWAWSHASVLLANDRKTQGTDNRAMGNIF
RDCLEGSLRKQGLMRSKLTEMVEKNVVPLTTQELVDILEEDIDFSDVI
AVELSEGSLDIESIFDGAPILWSAEVEEFGEGVVAVSYSSYYHLTLMD
QAAITMCAIMGKEGCRGLLTEKRCMAAIREQVRPFLIFLQIPEDSISW
VSDQFCDSRGLDEESTIMG* (SEQ ID NO: 295)

| SEQ | | SEQ | |
|---|---|---|---|
| ID NO | sequence | ID NO | sequence |
| 228 | MNLEVLCGRINVENG | 257 | KARSMDADIELQKSE |
| 229 | GRINVENGLSLGEPG | 258 | DIELQKSEEELEAIS |
| 230 | GLSLGEPGLYDQIYD | 259 | EEELEAISRALSFFS |
| 231 | GLYDQIYDRPGLPDL | 260 | SRALSFFSLFEPNIE |
| 232 | DRPGLPDLDVTVDAT | 261 | SLFEPNIERVEGTFP |
| 233 | LDVTVDATGVTVDIG | 262 | ERVEGTFPNSEIEML |
| 234 | TGVTVDIGAVPDSAS | 263 | PNSEIEMLEQFLSTP |
| 235 | GAVPDSASQLGSSIN | 264 | LEQFLSTPADVDFIT |
| 236 | SQLGSSINAGLITIQ | 265 | PADVDFITKTLKAKE |
| 237 | NAGLITIQLSEAYKI | 266 | TKTLKAKEVEAYADL |
| 238 | QLSEAYKINHDFTFS | 267 | EVEAYADLCDSHYLK |
| 239 | INHDFTFSGLSKTTD | 268 | LCDSHYLKPEKTIQE |
| 240 | SGLSKTTDRRLSEVF | 269 | KPEKTIQERLEINRC |
| 241 | DRRLSEVFPITHDGS | 270 | ERLEINCREAIDKTQ |
| 242 | FPITHDGSDGMTPDV | 271 | CEAIDKTQDLLAGLH |
| 243 | SDGMTPDVIHTRLDG | 272 | QDLLAGLHARSNKQT |
| 244 | VIHTRLDGTIVVVEF | 273 | HARSNKQTSLNRGTV |
| 245 | GTIVVVEFSTTRSHN | 274 | TSLNRGTVKLPPWLP |
| 246 | FSTTRSHNIGGLEAA | 275 | VKLPPWLPKPSSESI |
| 247 | NIGGLEAAYRTKIEK | 276 | PKPSSESIDIKTDSG |
| 248 | AYRKTIEKYRDPSIR | 277 | IDIKTDSGFGSLMDH |
| 249 | KYRDPISRRVDIMEN | 278 | GFGSLMDHGAVGELW |
| 250 | RRVDIMENPRVFFGV | 279 | HGAYGELWAKCLLDV |
| 251 | NPRVFFGVIVVSSGG | 280 | WAKCLLDVSLGNVEG |
| 252 | VIVVSSGGVLSNMPL | 281 | VSLGNVEGVVSDPAK |
| 253 | GVLSNMPLTQDEAEE | 282 | GVVSDPAKELDIAIS |
| 254 | LTQDEAEELMYRFCI | 283 | KELDIAISDDPEKDT |
| 255 | ELMYRFCIANEIYTK | 284 | SDDPEKDTPKEAKIT |
| 256 | IANEIYTKARSMDAD | 285 | TPKEAKITYRRFKPA |

Validation of Immunogenicity of Five SFTS Virus Antigens and Two Adjuvants

For vaccination. BALB/c mice were divided into the following five groups, each consisting of 6 mice: a naïve group; a group injected intramuscularly with a DNA vaccine: a group injected intramuscularly with a DNA vaccine and then subjected to electroporation: a group injected intramuscularly with a DNA vaccine and an IL-7 adjuvant and then subjected to electroporation; and a group injected intramuscularly with a DNA vaccine and an IL-33 adjuvant and then subjected to electroporation. Each mouse of the naïve group was vaccinated with 200 μg of a plasmid into which no SFTS virus gene was inserted, and each mouse of each of the other four groups was vaccinated with a total of 200 μg (40 μg for each DNA) of five SFTS virus antigen-expressing DNAs (DNA sequences of Gn, Gc, NP, NS and RdRp, which correspond to the DNA sequences of SEQ ID NOs: 286, 288, 290, 292 and 294, respectively, and to the amino acid sequences of SEQ ID NOs: 287, 289, 291, 293 and 295, respectively). In addition, each mouse of the groups to be vaccinated with IL-7 and IL-33 adjuvants was vaccinated with 50 μg of each adjuvant in addition to each DNA. For the mice of three groups, except the naïve group and the group injected intramuscularly with the DNA vaccine, the vaccination site of each mouse was subjected to electroporation using an electroporator at 0.2 A immediately after intramuscular injection. 21 days after the first vaccination, the second vaccination was performed using the same amount of the DNA. 21 days after the second vaccination, the mice were sacrificed, and the spleens and inguinal lymph nodes were isolated and used for immunogenicity evaluation.

Validation of T-Cell Immune Response to SFTSV Vaccine Candidate

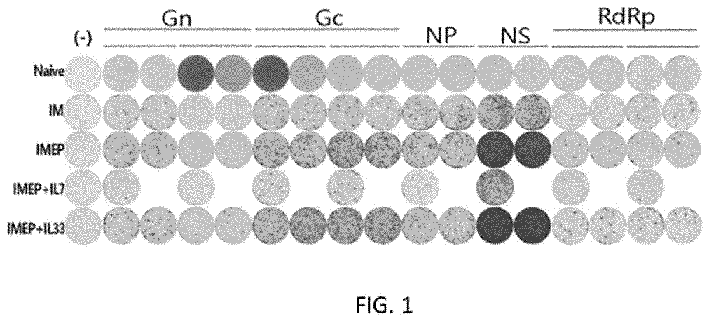

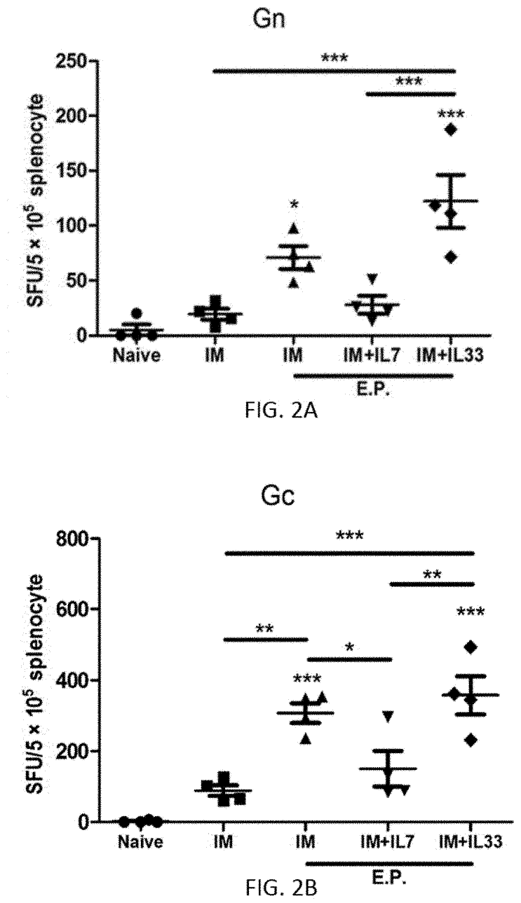

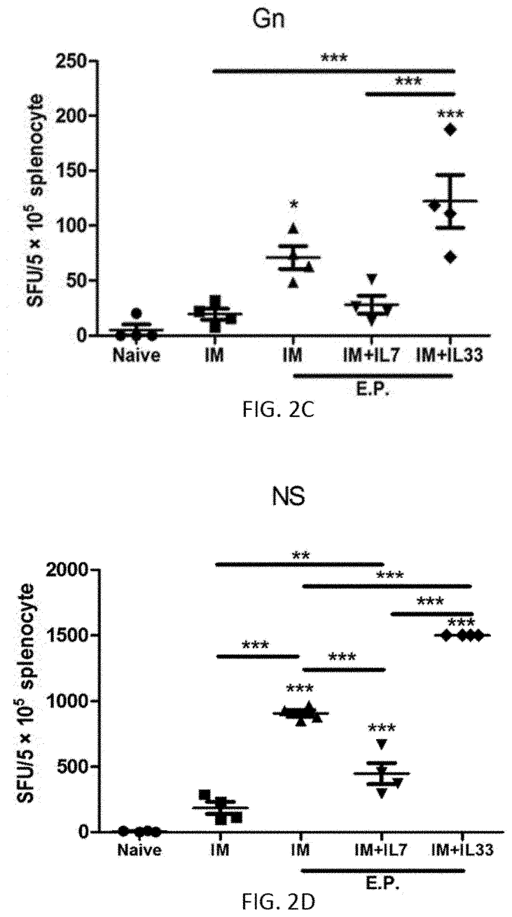

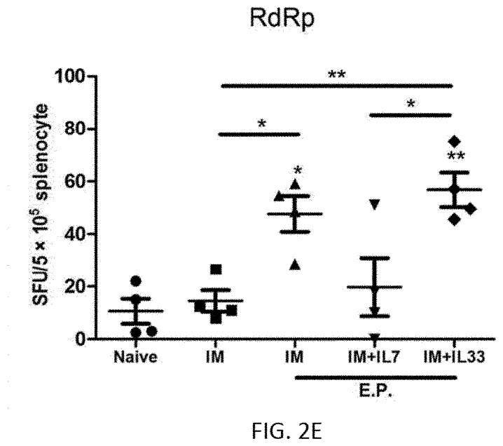

ELISpot assay was performed to measure T-cell response specific to SFTS virus (FIG. 1). 100 μl of an anti-human IFN-γ antibody (2 μg/ml; endogen) diluted in PBS was dispensed into each well of a 96-well filtration plate and incubated overnight at 4° C. Then, mouse spleen cells (5×10¹ cells/well) were incubated at 37° C. for 24 hours while the cells were stimulated with eight OLPs prepared from the peptides of SFTS virus. Then, the plate was washed, and 100 μl of biotinylated anti-human IFN-γ antibody (0.5 μg/ml; endogen) diluted in PBS/Tween 20/1% BSA was dispensed into each well and incubated overnight at 4° C. After washing four times, 100 μl of streptavidin-alkaline phosphatase (BD) diluted at 1:5.000 in PBS/Tween 20/1% BSA was dispensed into each well and incubated at 37° C. for 1 hour. Using an AP conjugate substrate kit (BIO-RAD), a reaction was performed for 10 minutes and stopped by washing, and then SFTS virus-specific production of IFN-γ by T-cells in response to the SFTS virus antigen was detected by ImmimoSpot (Cellular Technology Limited). Thereby, it was confirmed that T-cell immune response specific to SFTS virus was successfully induced in the mouse models after DNA vaccination. This immune response could be clearly observed in the group subjected to electroporation after intramuscular injection of the DNA vaccine and the group injected with the IL-33 adjuvant in addition to the DNA vaccine. In particular, it was confirmed that the immune response of T cells significantly increased in the group injected with the IL-33 adjuvant together with the SFTS virus antigen-expressing DNA vaccine (FIGS. 2A to 2E).

Evaluation of Multifunctionality of T Cells Induced by SFTSV DNA Vaccine Candidate

The spleen cells isolated from the vaccinated mice were stimulated with each of the SFTS virus OLPs. and then analyzed by intracellular cytokine staining (ICS) using multicolor.

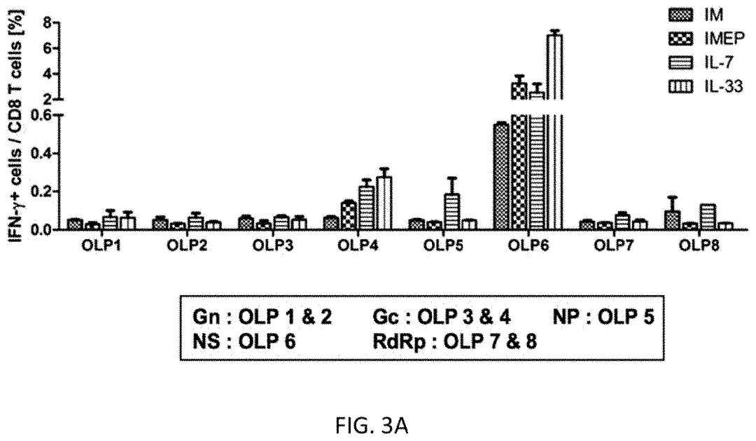

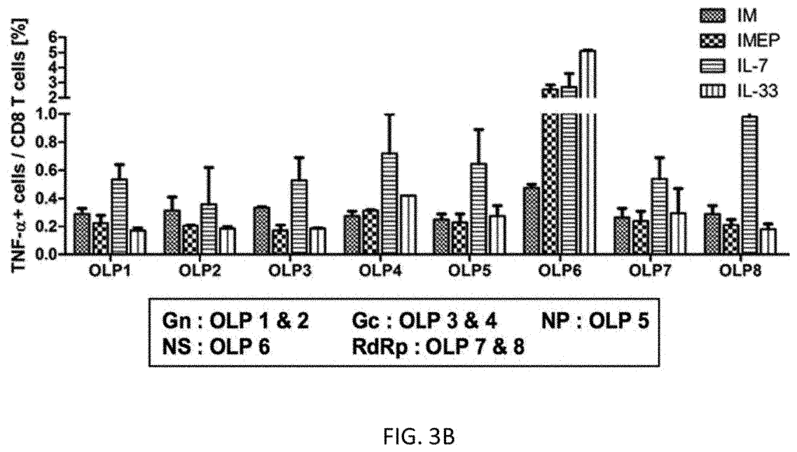

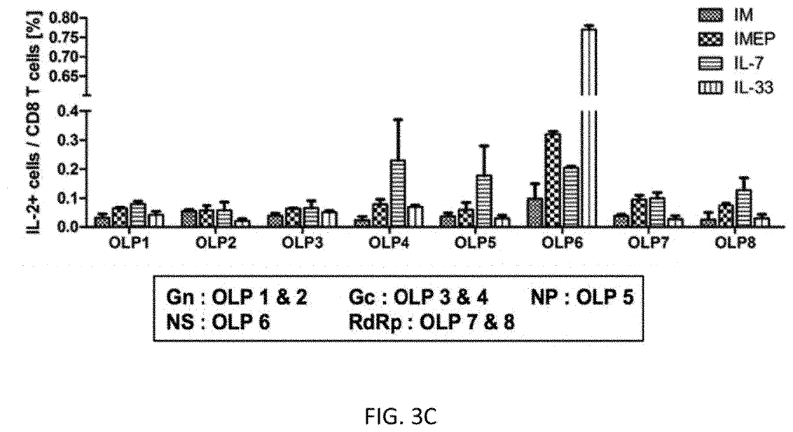

The cells were stimulated with each of the eight SFTS virus OLPs shown in Tables to 5 above, and were sorted into T-cell subsets secreting IFN-γ, TNF-α and IL-2, respectively. The proportion of T-cells secreting each cytokine was determined and the results are shown in FIGS. 3A to 3C.

As shown in FIGS. 3A to 3C, it was confirmed that a higher immune response generally occurred in the group (IMEP) subjected to both intramuscular injection and electroporation than in the group (IM) subjected to intramuscular injection alone. In addition, it was confirmed that the strongest immune response tended to occur in the group (IL-33) injected with IL-33 as an adjuvant in addition to being subjected to electroporation. This tendency was better identified in CD8+ T cells (FIGS. 3A to 3C).

It was confirmed that OLP6, an OLP corresponding to the NS protein of SFTS virus, induced the strongest immune response, and in particular, a very strong immune response appeared in cells secreting IFN-γ and TNF-α. In addition, it could be very clearly confirmed in the CD8+ T cells treated with OLP6 that the IMEP group showed a stronger immune response than the IM group, and that the strongest immune response was induced when IL-33 was used as an adjuvant.

This means that, when electroporation and IL-33 are used, the proportion of SFTS virus-specific T-cells induced by the vaccine is further increased.

Analysis of Multifunctionality of T-Cells Induced by Vaccine Candidate

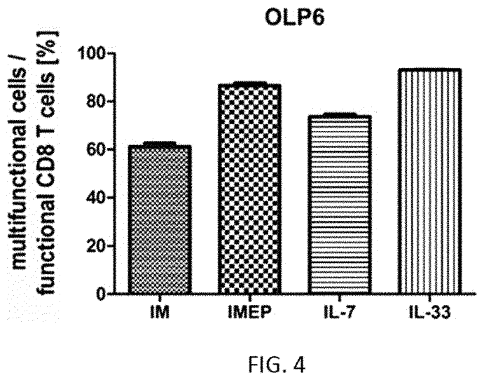

Based on FACS data, the multifunctionality of T-cells in each group was analyzed. Based on the results of FACS, the multifunctionality of CD8+ T cells stimulated with OLP6 inducing the strongest immune response was summarized for each group and the results were recorded. The results indicated that the proportion of mulifunctional T cells was higher in the IMEP group than in the IM group and was the highest in the group injected with IL-33 as an adjuvant in addition to being subjected to electroporation. This means that the T-cell immune response induced by the vaccine was qualitatively better when electroporation and IL-33 are used. This tendency also appeared in the proportion of multifunctional T cells among total effector T cells (FIG. 4). In addition. CD4+ T cells showed no significant difference in multifunctionality.

Evaluation of Antibody Formation Ability of SFTSV Vaccine Candidate

Verification of SFTSV-Specific Antibody Production Response Induced by Vaccine

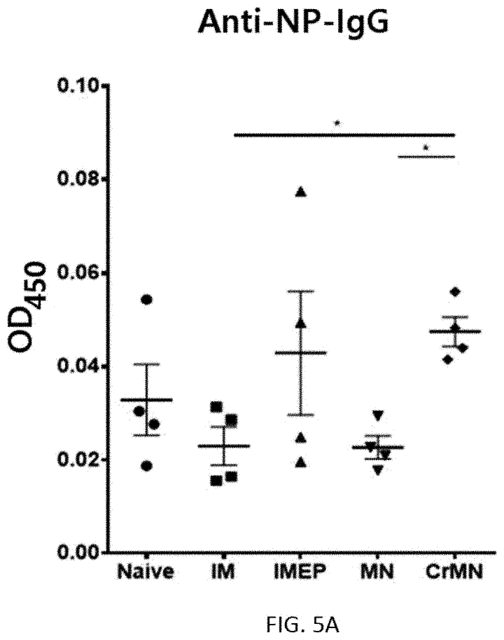

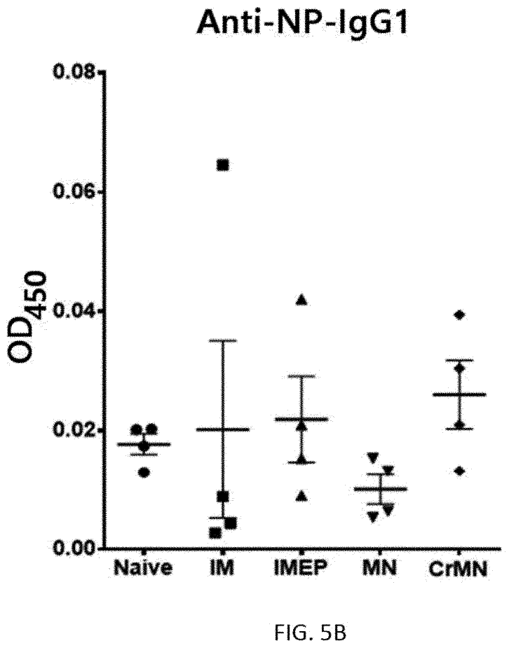

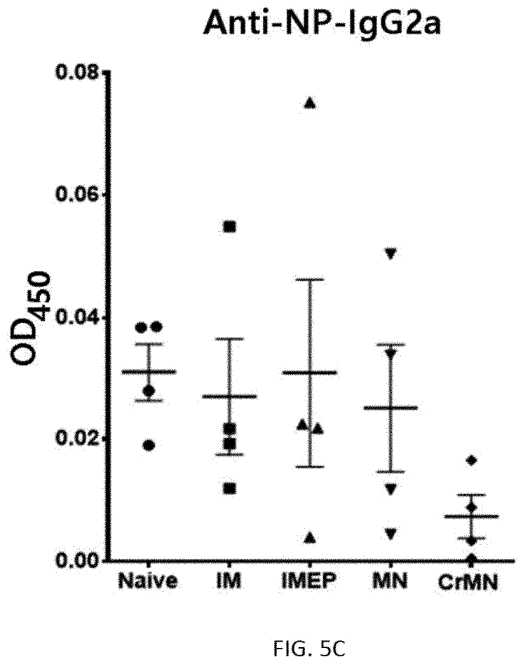

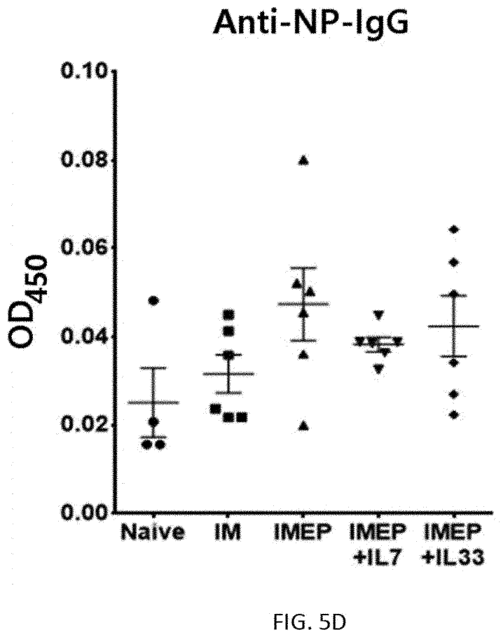

Enzyme-linked immunosorbent assay (ELISA) was performed to measure an SFTS virus-specific antibody production response induced by SFTSV vaccine. In order to establish an ELISA technique for the recombinant SFTSV NP antigen protein, an experiment was conducted using the serum of mice evaluated to have vaccine-induced immunogenicity. Using the ELISA assay technique as described above, the antibody immune response induced by the vaccine was quantitatively analyzed. As shown in FIGS. 5A to 5F, it was confirmed that the antibody immune response generated in the mice of the intramuscular injection+electroporation (IMEP) group was stronger than those in other groups. In FIGS. 5A to 5F, CrMN represents a nano-pattern formed on the surface of a microneedle (MN) by treating the microneedle surface with a chromium precursor.

Quantitative Evaluation of Neutralizing Antibody Titer Induced by SFTSV DNA Vaccine

The neutralizing antibody titers of 33 antibodies produced in the mice by the DNA vaccine candidates and various adjuvants were measured by PRNT50 assay.

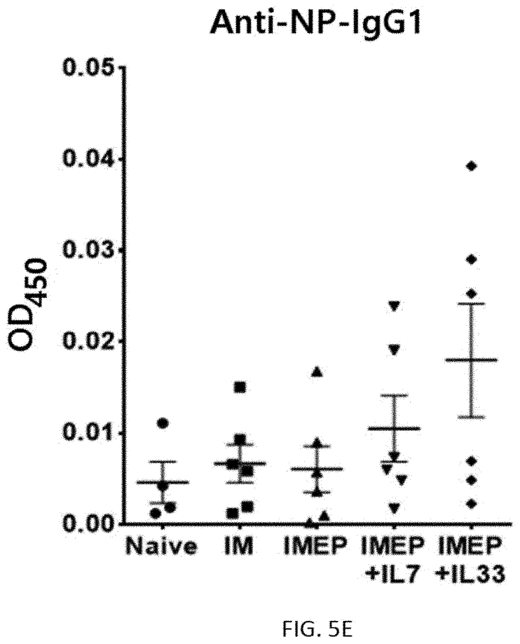

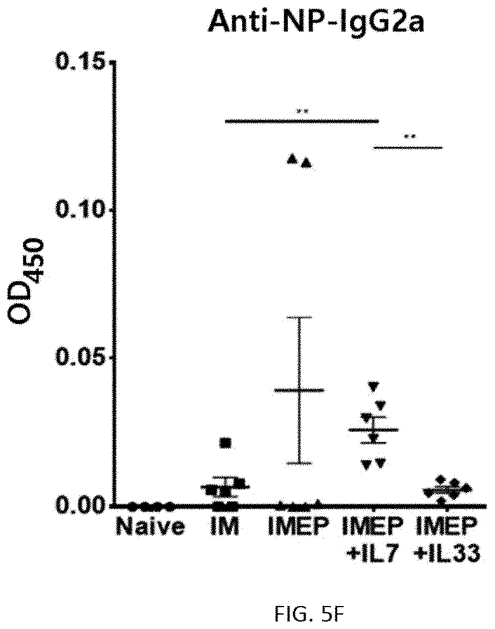

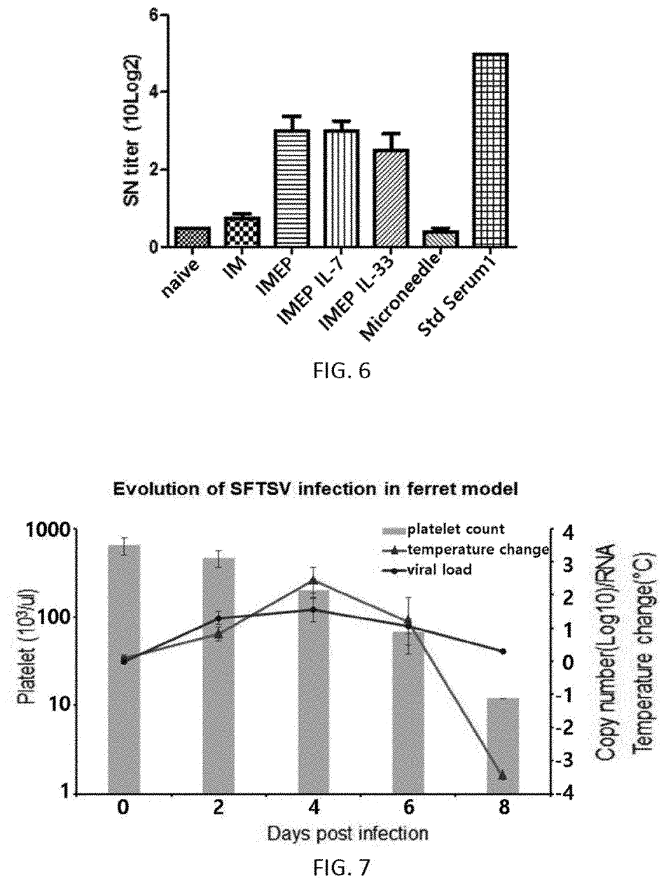

The mouse standard antibody developed by the present inventors was used as a positive control. The experimental results indicated that the animals of the test groups showed an SN titer of 20 to 160, suggesting that a neutralizing antibody was formed. In particular, a stronger neutralizing antibody response was observed in the group (IMEP) injected with the DNA vaccine by intramuscular injection+electroporation, and a very weak neutralizing antibody titer was detected in the microneedle group (Microneedle) (FIG. 6).

Establishment of Medium-Sized Animal Models as SFTSV-Infected Animal Models

New animal models for validating the infection inhibitory effect of the SFTSV vaccine were developed. As a result of infecting medium-sized animals with SFTSV isolated from an SFTS patient, it was observed that the virus was detected in the blood and that the platelet count continued to decrease up to day 8 after infection (FIG. 7). In addition, it was confirmed that the body temperature increased by 2° C. or more on day 4, and as this symptom persisted, and all the infected animals died about 9 days after infection. These results were very similar to the clinical courses of the patient. When the animals do not enter the recovery phase after SFTSV infection, they tended to die after about 10 days. The medium-sized animal models established by the present inventors showed very similar clinical findings to those of SFTS patients, such as high fever, increased viral load, platelets and changes in blood components when infected with SFTSV, and thus were determined to show excellent suitability as SFTSV-infected animal models for vaccine efficacy validation.

Validation of Immunogenicity of SFTSV Vaccine Using SFTSV-Infected Animal Models

The present inventors used the established SFTSV-infected medium-sized animal models as models to validate the infection inhibitory effect of the SFTSV vaccine. Each animal of a vaccination group (N=6) was vaccinated with a total of 1 mg (200 μg for each DNA) of the five SFTS virus antigen-expressing DNAs (see Table 6 below) by intradermally injecting the DNAs into both femurs in an amount of 500 μg for each femur. Each animal of a control group was vaccinated with 1 mg of a mock plasmid (derived from pVax-1), into which no SFTS virus gene was inserted, by intradermally injecting the DNAs into both femurs in an amount of 500 μg for each femur. Both the two groups were subjected to electrophoresis using an electroporator at 0.2 A immediately after intradermal injection. The vaccination was performed a total of 5 times, once every two weeks (vaccinated on days 0, 14, 28, 42 and 56).

TABLE 6

| | Control | Vaccination |
|---|---|---|
| pVax1 | 1 mg | |
| pGX27-Gn | | 200 ug |
| pGX27-Gc | | 200 ug |
| pGX27-NP | | 200 ug |
| pGX27-NSs | | 200 ug |
| pGX27-RdRp | | 200 ug |

Evaluation of T-Cell Immune Response Induced by Vaccine in SFTSV-Infected Medium-Sized Animal Models

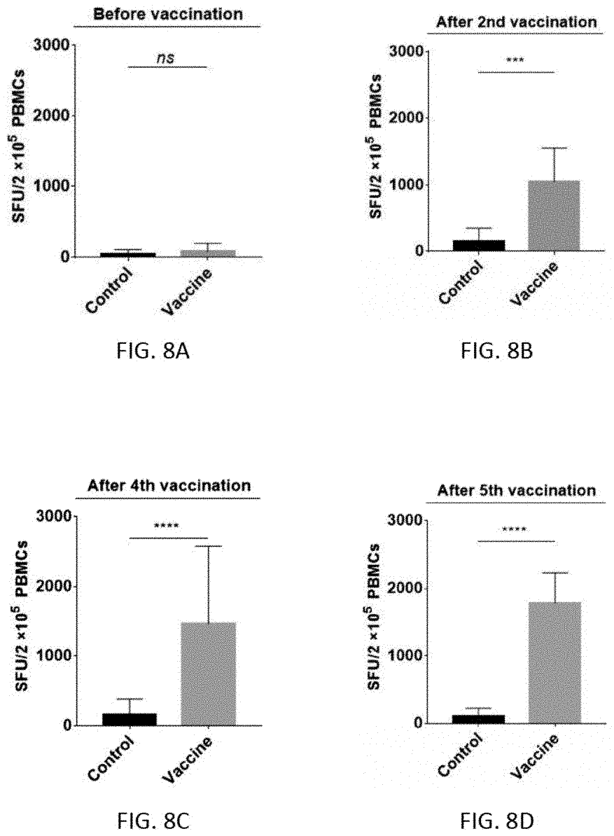

Before vaccination and 2 weeks after each of 2^{nd} vaccination, 4^{th} vaccination and 5^{th} vaccination, 5 ml of blood was sampled, PBMCs and serum were isolated therefrom, and SFTSV-specific T cell immune response (ELISpot assay) and antibody immune response (ELISA and neutralizing antibody assay) were measured. SFTSV-specific T cell immune response induced by the vaccine was evaluated by ELISpot assay. As shown in FIGS. 8A to 8D, it was confirmed that the SFTSV vaccine candidate induced very strong SFTSV-specific T cell immune response in the medium-sized animals, and that stable immune response was observed after 2^{nd} vaccination.

Quantitative Evaluation of Antibody Immune Response and Neutralizing Antibody Titer Induced by Vaccine in SFTSV-Infected Medium-Sized Animal Models

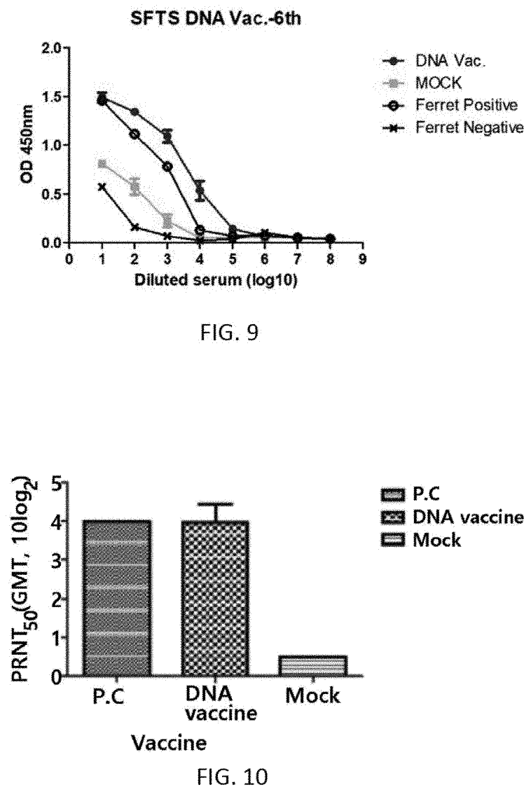

The formation of SFTSV-specific reactive antibody by the DNA vaccine was measured and evaluated by ELISA assay. As shown in FIG. 9, it was confirmed that the mock group (n=6) vaccinated with the empty vector vaccine showed an ELISA value similar to that of the serum of the negative control, indicating that no specific antibody was formed in the mock group. However, it was observed that the production of SFTSV-specific reactive antibody in the group (n=6) vaccinated with the SFTSV DNA vaccine was similar to or higher than that in the serum of the positive control. This result suggests that the vaccine candidate can effectively induce antibody immune response in medium-sized animals.

The neutralizing antibody titer of the antibody induced by the DNA vaccine was measured by PRNT50 assay. As shown in FIG. 10, it was confirmed that the titer of neutralizing antibody produced in the six animals vaccinated with the SFTSV DNA vaccine was similar to that in the positive control group. In contrast, it could be confirmed that no neutralizing antibody was produced in the group (n=6) vaccinated with the empty vector vaccine. These results suggest that SFTSV-specific neutralizing antibody can be effectively induced by the DNA vaccine candidate.

Evaluation of Preventive Effect of SFTSV Preventive DNA Vaccine in SFTSV-Infected Medium-Sized Animal Models

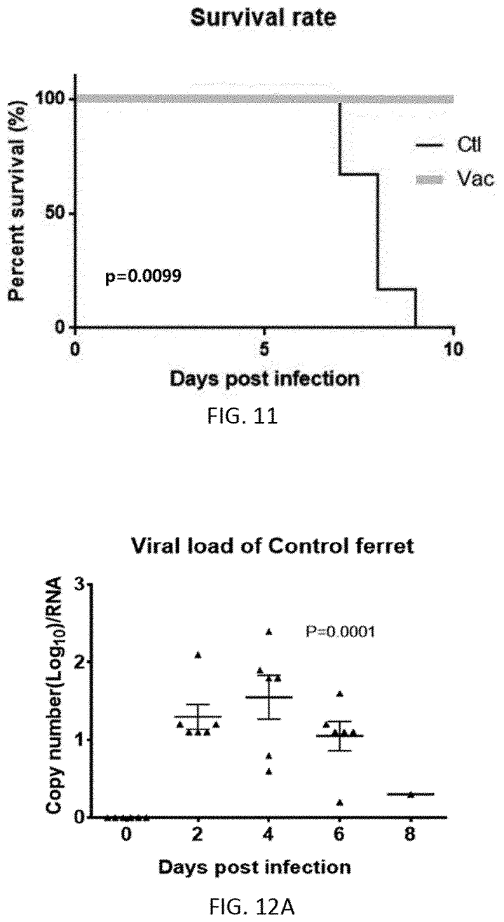

To evaluate the preventive effect of the SFTSV vaccine the animals vaccinated with SFTSV vaccine were infected with a lethal dose of SFTSV, and then evaluated for clinical symptoms such as survival rate, SFTSV viral load, platelet count, and body temperature and body weight changes. As shown in FIG. 11, as a result of evaluating survival rate, it was confirmed that the six control animals all died (2 animals on day 7, 3 animals on day 8, and 1 animal on day 9 after infection), whereas the six animals vaccinated with the SFTSV vaccine all survived.

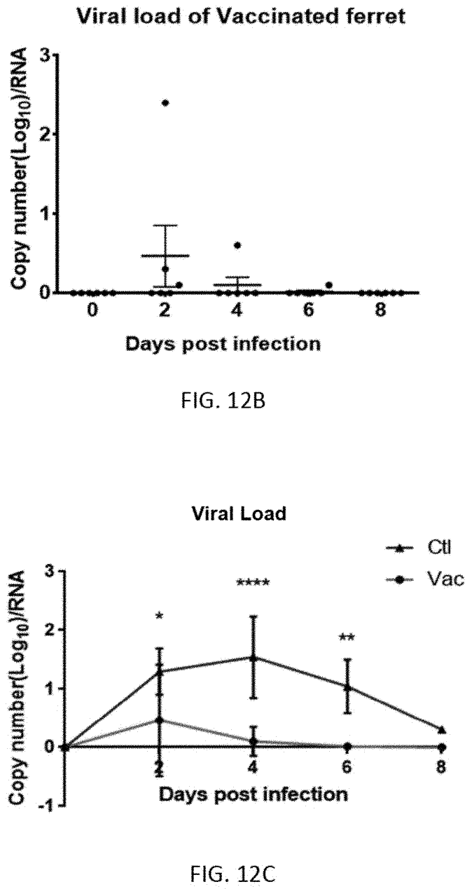

SFTSV viral load was measured by real-time PCR. As shown in FIGS. 12A to 12C, it was observed that the viral load in the control group increased on day 2 after infection and was the highest on day 4 after infection. In comparison with this, no viral load was detected in four medium-sized animals of the vaccinated group. It was confirmed that, in one animal of the vaccinated group, a viral load similar to that in the control group was detected, but decreased on day 4 after infection and then was completely removed on day 6 after infection.

It was observed that this animal was the same animal as the animal whose platelets have increased, and as the viral load therein decreased, the platelet count thereof was also returned to normal.

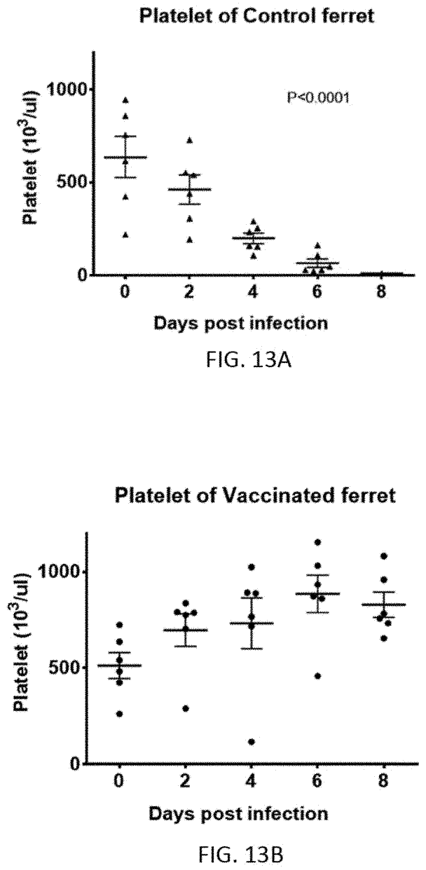

As a result of counting platelets, it was observed that the platelet count of the control group was decreased rapidly by SFTSV infection (FIGS. 13A to 13C). In comparison with this, it was confirmed that the platelet count of the vaccinated group was maintained normally. It was observed that, in one animal of the vaccinated group, the platelet count decreased to about 120×10³/μl on day 4 after infection, but it was confirmed that the platelet count was returned to normal on day 6 after infection.

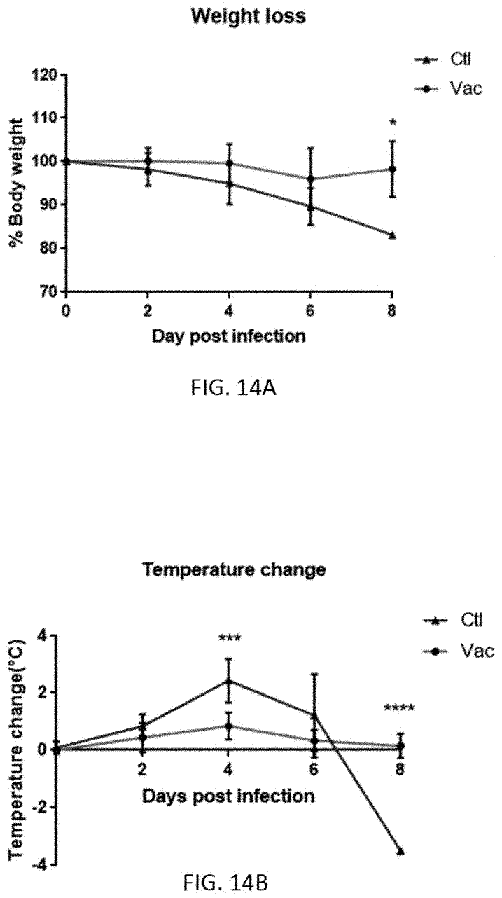

Meanwhile, as shown in FIG. 14A, it was confirmed that the animals of the control group showed a significant loss in body weight after SFTSV infection, but the animals of the vaccinated group showed no loss in body weight. In addition, as shown in FIG. 14B, the animals of the control group showed an increase in body temperature of about 2° C. after SFTSV infection, but no apparent change in body temperature was observed in the animals of the vaccinated group.

Taking these results together, it could be seen that the SFTSV-preventive DNA vaccine candidate developed by the present inventors could effectively prevent SFTSV infection, as confirmed through verification of various clinical indicators (survival rate, platelet count, body temperature, and body weight) in the medium-sized animal models.

Evaluation of Cross-Reactivity of Neutralizing Antibody Induced in Mice and Medium-Sized Animals

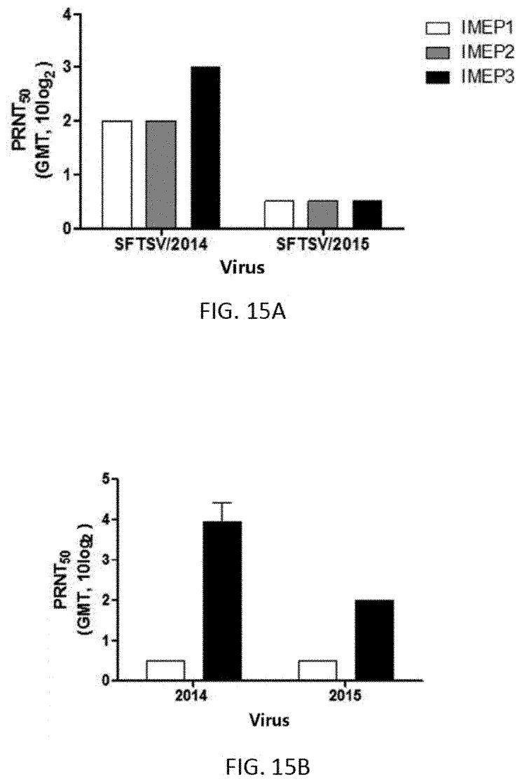

To evaluate the cross-reactivity of SFTSV neutralizing antibody produced in mice after SFTSV DNA vaccination, a PRNT50 test was performed using other SFTS viruses. As shown in FIG. 15A, a neutralizing antibody against SFTSV/2014 virus was produced at a titer of about 40 to 80, whereas the production of a neutralizing antibody against another virus SFTSV/2015 did not clearly appear.

In addition, to evaluate the cross-reactivity of SFTSV neutralizing antibody produced in medium-sized animals after SFTSV DNA vaccination, a PRNT50 test was performed using other SFTS viruses. As shown in FIG. 15B, a neutralizing antibody against SFTSV/2014 virus was produced at a titer of about 160 to 320, which was about two times higher than the titer value against another virus SFTSV/2015.

Evaluation of Immunogenicity of SFTSV Preventive DNA Vaccine in SFTSV-Infected Medium-Sized Animal Models

Using the SFTSV-infected medium-sized animal models established as described above, the infection preventive effect of the SFTSV vaccine was evaluated. To evaluate the preventive effect of each vaccine, SFTSV-vaccinated groups (N=4, N=3, N=3, and N=3, respectively) were vaccinated respectively with 1 mg of a Gn/Gc vaccine, an NP vaccine, an NS vaccine and an RdRp vaccine, which are SFTS virus antigen-expressing DNAs, by intradermally injecting each DNA into both femurs. A control group was vaccinated with 1 mg of a mock plasmid (derived from pVax-1), into which no SFTS virus gene was inserted, by intradermally injecting the plasmid into both femurs (500 μg for each femur). Both the vaccinated groups and the control group were subjected to electroporation using an electroporator at 0.2 A immediately after intradermal injection. The vaccination was performed a total of three times, once every two weeks (vaccinated on days 0, 14 and 28).

Evaluation of T-Cell Immune Response Induced by Vaccine In SFTSV-Infected Medium-Sized Animal Models

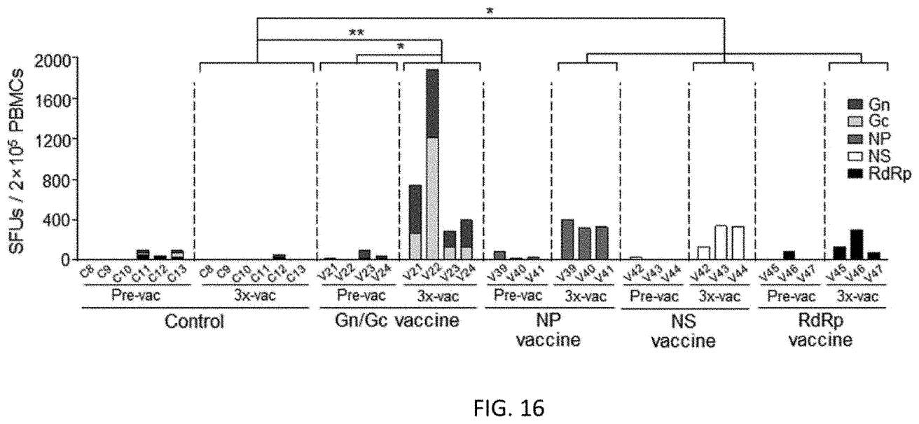

Before vaccination and 2 weeks after 3_{rd} vaccination, 5 ml of blood was sampled. PBMCs and serum were isolated therefrom, and SFTSV-specific T cell immune response (ELISpot assay) and antibody immune response (ELISA and neutralizing antibody assay) were measured. SFTSV-specific T cell immune response induced by the vaccine was evaluated by ELISpot assay. It was confirmed that a very strong SFTSV-specific T cell immune response against each SFTSV antigen depending on the kind of vaccine was induced by the SFTSV vaccine candidate in the medium-sized animal models. As shown in FIG. 16, the highest SFTSV-specific immune response could be detected in the group treated with the Ga/Gc SFTSV vaccine.

Quantitative Evaluation of Antibody Immune Response and Neutralizing Antibody Titer Induced by Vaccine in SFTSV-Infected Medium-Sized Animal Models

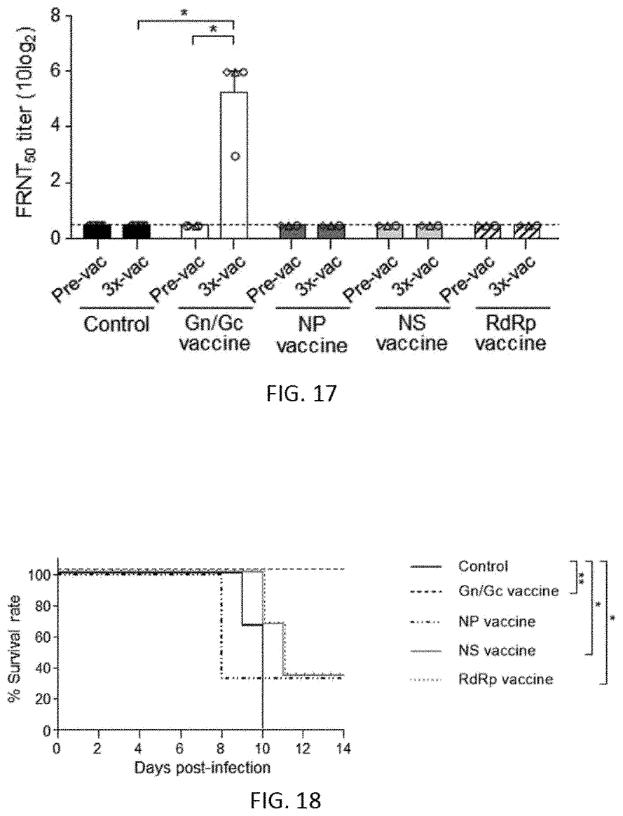

The neutralizing antibody titer of antibody induced by the DNA vaccine was measured by PRNT50 assay. As shown in FIG. 17, it was confirmed that neutralizing antibody titer was effectively produced in the four animals vaccinated with the DNA vaccine against SFTSV Gu/Gc. In contrast, it could be confirmed that no neutralizing antibody was produced in the animals of the groups vaccinated with the vaccines against NP, NS and RdRp, respectively, including the group (n=6) vaccinated with the empty vector vaccine. These results mean that SFTSV-specific neutralizing antibody can be effectively induced by the DNA vaccine candidate against Gn/Gc.

Evaluation of Preventive Effect of SFTSV Preventive DNA Vaccine in SFTSV-Infected Medium-Sized Animal Models

To evaluate the preventive effect of each vaccine, the animals vaccinated with SFTSV vaccines against Gn/Gc, NP, NS and RdRp, respectively, were infected with a lethal dose of SFTSV, and then evaluated for clinical symptoms such as survival rate, SFTSV viral load, platelet count, body temperature and body weight changes, and ALT and AST changes.

As shown in FIG. 18, as a result of evaluating survival rate, it was confirmed that, in the control group, the six animals all died after infection, and in each of the three groups vaccinated with the SFTSV vaccines against NP, NS and RdRp, respectively, only one of the three animals survived, whereas the four animals vaccinated with the SFTSV vaccine against Gn/Gc all survived.

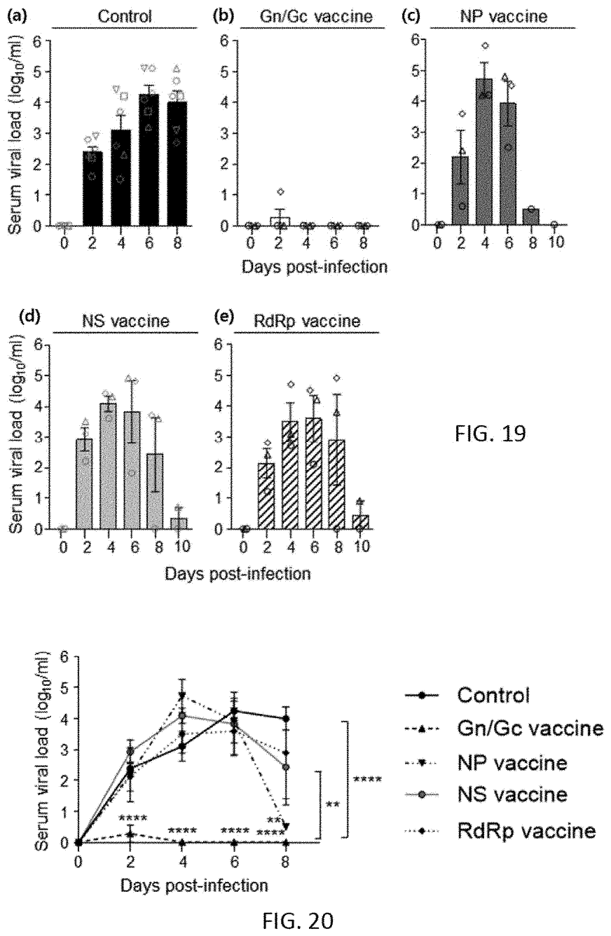

In addition, SFTSV viral load was measured by real-time PCR. The results are shown in FIGS. 19 and 20. It was observed that the viral load in the control group increased on day 2 after infection and was the highest on day 6 after infection. In comparison with this, no viral load was detected in four medium-sized animals of the Gn/Gc vaccine group. It was confirmed that, in one animal of the G/Gc vaccine group, a very low viral load was detected on day 2 after infection, but was completely removed on day 4 after infection. However, it was confirmed that the viral loads in the three groups vaccinated with the SFTSV vaccines against NP. NS and RdRp, respectively, increased to levels similar to the viral load of the control group.

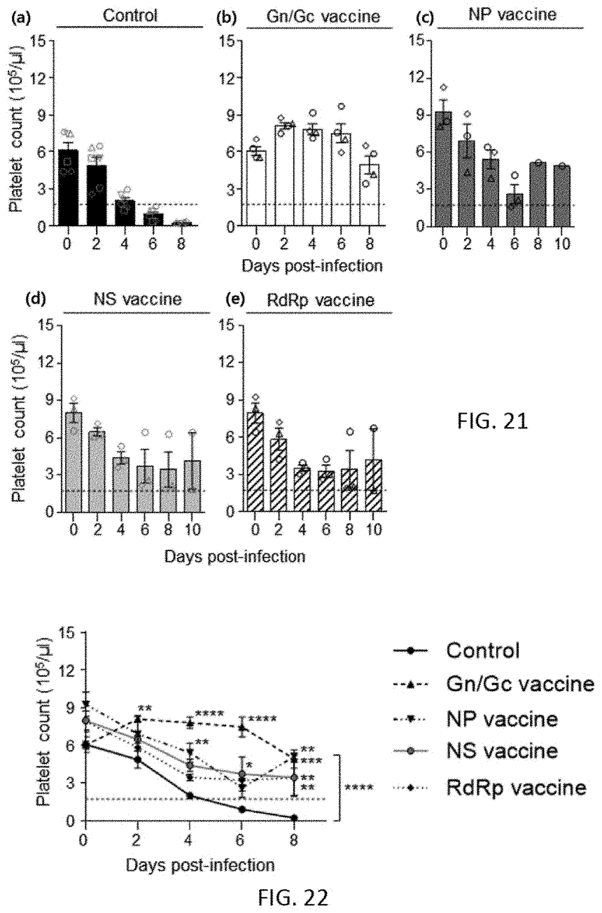

In addition, as shown in FIGS. 21 and 22, as a result of counting platelets, it was observed that the platelet count of the control group was decreased rapidly by SFTSV infection. In comparison with this, it was confirmed that the platelet count of the Gn/Gc vaccine group was maintained normally. However, in the three groups vaccinated with the SFTSV vaccines against NP, NS and RdRp, respectively, it was observed that the platelet count decreased to about 300×10³/μl up to day 6 after infection.

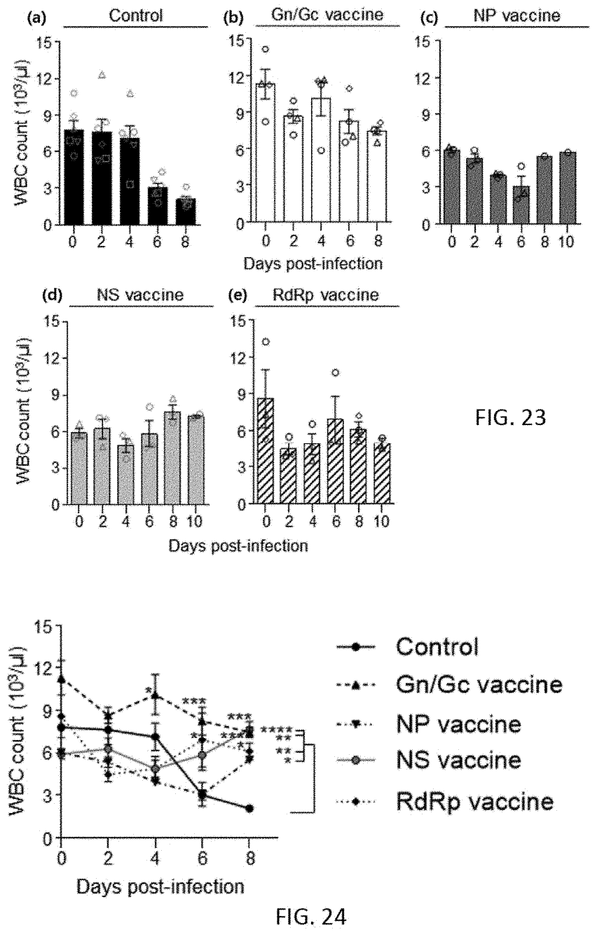

As shown in FIGS. 23 and 24, as a result of counting white blood cells, it was observed that white blood cell count of the control group was decreased rapidly by SFTSV infection. In comparison with this, it was confirmed that the white blood cell count of the Gn/Gc vaccine group was maintained normally. On the other hand, it was observed that, in the three groups vaccinated with the SFTSV vaccines against NP, NS and RdRp, respectively, the white blood cell count decreased up to days 6 or 4 after infection.

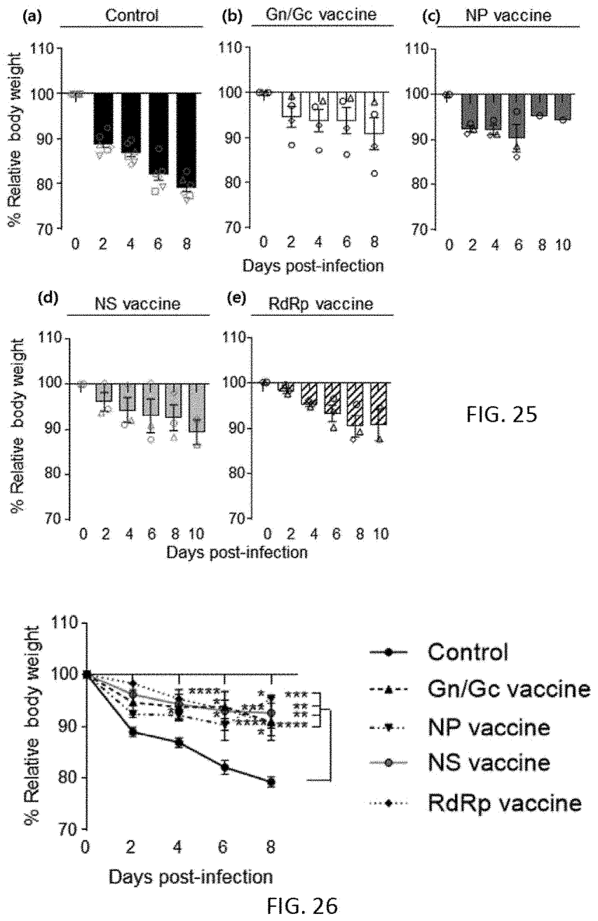

Meanwhile, it was confirmed that, after SFTSV infection, the animals of the control group showed a significant loss in body weight (relative body weight of 80% or less), whereas the animals of the vaccine groups showed no loss in body weight (relative body weight of 90% or less) (see FIGS. 25 and 26).

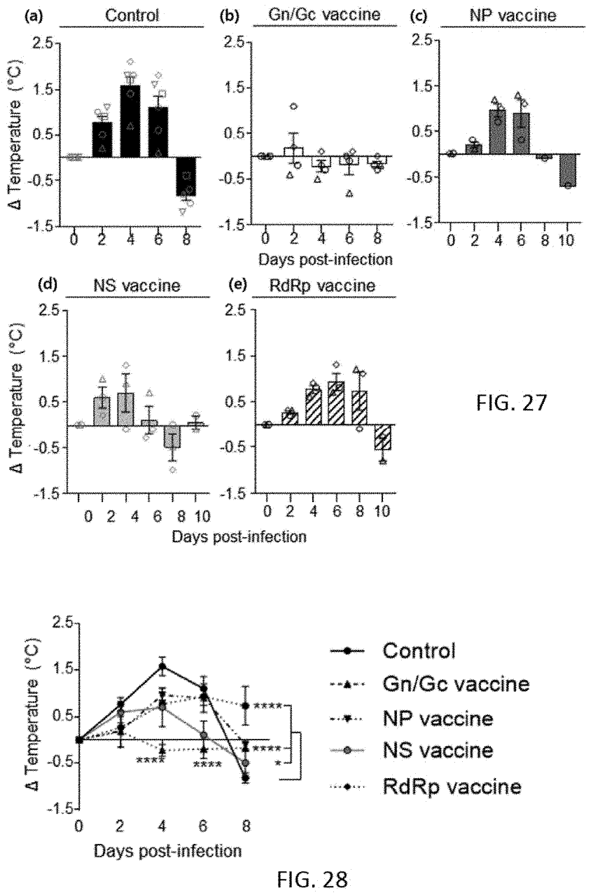

In addition, as shown in FIGS. 27 and 28, after SFTSV infection, the animals of the control group showed an increase in body temperature of about 2° C., but no apparent change in body temperature was observed in the animals of the Gn/Gc vaccine group. However, it was observed that the three groups vaccinated with the SFTSV vaccines against NP, NS and RdRp, respectively, showed an increase in body temperature of about 0.5 to 1° C.

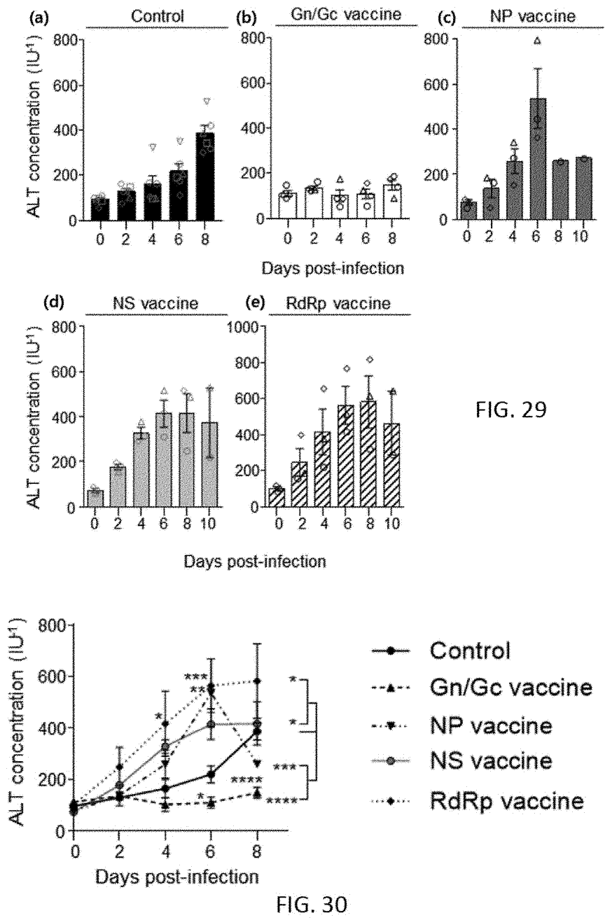

As shown in FIGS. 29 and 30, as a result of measuring serum ALT concentration, it was observed that the ALT concentration in the control group was increased rapidly by SFTSV infection. In comparison with this, the ALT concentration in the Gn/Gc vaccine group was maintained normally. On the other hand, it was observed that, in the three groups vaccinated with the SFTSV vaccines against NP. NS and RdRp, respectively, the ALT concentration increased rapidly up to day 6 after infection.

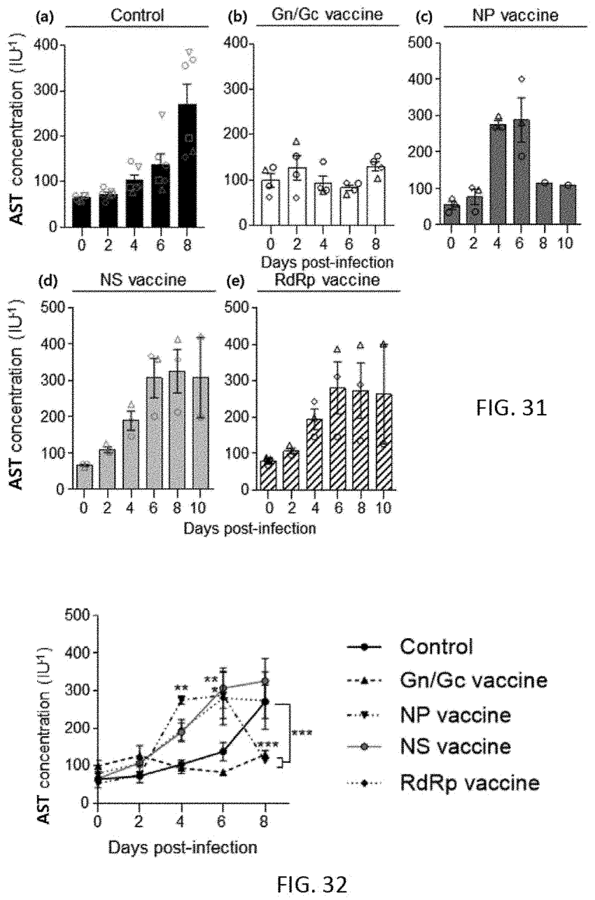

In addition, as shown in FIGS. 31 and 32, as a result of measuring serum AST concentration, it was observed that the AST concentration in the control group was increased rapidly by SFTSV infection. In comparison with this, it was confirmed that the AST concentration in the Gn/Gc vaccine group was maintained normally. On the other hand, it was observed that, in the three groups vaccinated with the SFTSV vaccines against NP. NS and RdRp, respectively, the AST concentration increased rapidly up to day 6 after infection.

As a result of evaluating the preventive effect of each of the Gn/Gc, NP, NS and RdRp antigens in the infected medium-sized animal (ferret) models as described above, it could be confirmed that the Gn/Gc DNA vaccine exhibited a significantly higher protective effect than other antigen-expressing DNA vaccines.

Although the present disclosure has been described in detail with reference to the specific features, it will be apparent to those skilled in the art that this detailed description is only of a preferred embodiment thereof, and does not limit the scope of the present disclosure. Thus, the substantial scope of the present disclosure will be defined by the appended claims and equivalents thereto.

### INDUSTRIAL APPLICABILITY

The present disclosure relates to a vaccine composition for preventing or treating an infectious disease caused by severe fever with thrombocytopenia syndrome (SFTS) virus.

1. An antigenic composition comprising, as an active ingredient, any one or more selected from the group consisting of:
a first recombinant peptide which comprises an amino acid sequence represented by SEQ ID NO: 287, or which is encoded by a first recombinant DNA comprising a nucleotide sequence represented by SEQ ID NO: 286;
a second recombinant peptide which comprises an amino acid sequence represented by SEQ ID NO: 289, or which is encoded by a second recombinant DNA comprising a nucleotide sequence represented by SEQ ID NO: 288;
a third recombinant peptide which comprises an amino acid sequence represented by SEQ ID NO: 291, or which is encoded by a third recombinant DNA comprising a nucleotide sequence represented by SEQ ID NO: 290;
a fourth recombinant peptide which comprises an amino acid sequence represented by SEQ ID NO: 293, or which is encoded by a fourth recombinant DNA comprising a nucleotide sequence represented by SEQ ID NO: 292; and
a fifth recombinant peptide which comprises an amino acid sequence represented by SEQ ID NO: 295, or which is encoded by a fifth recombinant DNA comprising a nucleotide sequence represented by SEQ ID NO: 294.

2. The antigenic composition of claim 1, which is injected in vivo through a route selected from among intramuscular, intradermal, subcutaneous, subepidermal, transdermal and intravenous routes.

3. The antigenic composition of claim 1, which is injected into a subject through intramuscular injection.

4. The antigenic composition of claim 1, which is injected into a subject through intradermal injection.

5. The antigenic composition of claim 2, wherein the in vivo injection of the antigenic composition into a subject is followed by electroporation.

6. A vaccine comprising, as an active ingredient, any one or more selected from the group consisting of:
a first recombinant peptide which comprises an amino acid sequence represented by SEQ ID NO: 287, or which is encoded by a first recombinant DNA comprising a nucleotide sequence represented by SEQ ID NO: 286;
a second recombinant peptide which comprises an amino acid sequence represented by SEQ ID NO: 289, or which is encoded by a second recombinant DNA comprising a nucleotide sequence represented by SEQ ID NO: 288;
a third recombinant peptide which comprises an amino acid sequence represented by SEQ ID NO: 291, or which is encoded by a third recombinant DNA comprising a nucleotide sequence represented by SEQ ID NO: 290;
a fourth recombinant peptide which comprises an amino acid sequence represented by SEQ ID NO: 293, or which is encoded by a fourth recombinant DNA comprising a nucleotide sequence represented by SEQ ID NO: 292; and
a fifth recombinant peptide which comprises an amino acid sequence represented by SEQ ID NO: 295, or which is encoded by a fifth recombinant DNA comprising a nucleotide sequence represented by SEQ ID NO: 294.

7. The vaccine of claim 6, which is injected in vivo through a route selected from among intramuscular, intradermal, subcutaneous, subepidermal, transdermal and intravenous routes.

8. The vaccine of claim 6, which is injected into a subject through intramuscular injection.

9. The vaccine of claim 6, which is injected into a subject through intradermal injection.

10. The vaccine of claim 7, wherein the in vivo injection of the vaccine into a subject is followed by electroporation.

11. The vaccine of claim 6, further comprising an adjuvant.

12. The vaccine of claim 11, wherein the adjuvant is at least one of IL-7 and IL-33.

13. An expression vector comprising any one or more recombinant DNAs selected from the group consisting of:
a first recombinant DNA comprising a nucleotide sequence represented by SEQ ID NO: 286;
a second recombinant DNA comprising a nucleotide sequence represented by SEQ ID NO: 288;
a third recombinant DNA comprising a nucleotide sequence represented by SEQ ID NO: 290;
a fourth recombinant DNA comprising a nucleotide sequence represented by SEQ ID NO: 292; and
a fifth recombinant DNA comprising a nucleotide sequence represented by SEQ ID NO: 294.

14. A transformant obtained by introducing the expression vector of claim 13 into a host cell by transformation.

15. A method for preventing or treating severe fever with thrombocytopenia syndrome (SFTS) virus infection, the method comprising a step of administering to a subject an effective amount of the antigenic composition of claim 1.

16. A pharmaceutical composition for preventing or treating severe fever with thrombocytopenia syndrome (SFTS) virus infection, the pharmaceutical composition comprising, as an active ingredient, the antigenic composition of claim 1.

17. A method for preventing or treating severe fever with thrombocytopenia syndrome (SFTS) virus infection, the method comprising a step of administering to a subject an effective amount of the expression vector of claim 13.

18. A method for preventing or treating severe fever with thrombocytopenia syndrome (SFTS) virus infection, the method comprising a step of administering to a subject an effective amount of the transformant of claim 14.

19. The method of claim 15, wherein the antigenic composition is injected in vivo through a route selected from among intramuscular, intradermal, subcutaneous, subepidermal, transdermal and intravenous routes.

20. The method of claim 19, wherein the in vivo injection of the antigenic composition into a subject is followed by electroporation.
